SEQUENCE LISTING (17 sequences)

SEQ ID NO: 1 - DNA, 2291 bp, Homo sapiens
  CDS (138)..(1748)
ggaattacttgcagggctaacctagtgcctatagctaaggcaggtacctgcatccttgtttttgtttagtggatcctctatccttcagagactctggaacccctgtggtcttctcttcatctaatgaccctgaggggatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggtggtgccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagccca

SEQ ID NO: 2 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 3 - DNA, 2344 bp, Homo sapiens
  CDS (191)..(1801)
atcctgccttcagagtcttactgcgcggggccccagtctccagtcccgcccaggcgcctttgcaggctgcggtgggatttcgttttgcctccggttggggctgctgtttctcttcgccgacgtggatcctctatccttcagagactctggaacccctgtggtcttctcttcatctaatgaccctgaggggatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggtggtgccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagccca

SEQ ID NO: 4 - protein, 536 aa, Homo sapiens; identical to SEQ ID NO: 2

SEQ ID NO: 5 - DNA, 2325 bp, Homo sapiens
  CDS (172)..(1782)
atcctgccttcagagtcttactgcgcggggccccagtctccagtcccgcccaggcgcctttgcaggctgcggtgggatttcgttttgcctccggttggggctgctgtttctcttcgccgacgagactctggaacccctgtggtcttctcttcatctaatgaccctgaggggatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggtggtgccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagccca

SEQ ID NO: 6 - protein, 536 aa, Homo sapiens; identical to SEQ ID NO: 2

SEQ ID NO: 7 - DNA, 2161 bp, Homo sapiens
  CDS (269)..(1618)
atcctgccttcagagtcttactgcgcggggccccagtctccagtcccgcccaggcgcctttgcaggctgcggtgggatttcgttttgcctccggttggggctgctgtttctcttcgccgacggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggtggtgccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagccca

SEQ ID NO: 8 - protein, 449 aa, Homo sapiens
MELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 9 - DNA, 2144 bp, Homo sapiens
  CDS (138)..(1601)
ggaattacttgcagggctaacctagtgcctatagctaaggcaggtacctgcatccttgtttttgtttagtggatcctctatccttcagagactctggaacccctgtggtcttctcttcatctaatgaccctgaggggatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggtggtgccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagccca

SEQ ID NO: 10 - protein, 487 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 11 - DNA, 3959 bp, Homo sapiens
  CDS (403)..(1587)
agtgcgcggagctgcggcggtggagctgctcggccgggagcccggcggcgagacggagaggcggcggaggtgcccgcgcgcagggtctggtgggcggccgcgaggctcgggagaggcgaaccggagcgcgggaccgcggtcgccccgaccagagccgggagaccgcagcacccgcagccgcccgcgagcgcgccgaagacagcgcgcaggcgagagcgcgcgggcgggggcgcgcaggccctgcccgccccttccgtccccacccccctccgccctttcctctccccaccttcctctcgcctcccgcgcccccgcaccgggcgcccaccctgtcctcctcctgcgggagcgttgtccgtgttggcggccgcagcgggccgggccggtccggcgggccgggggatggcgctgctggacctggccttggagggaatggccgtcttcgggttcgtcctcttcttggtgctgtggctgatgcatttcatggctatcatctacacccgattacacctcaacaagaaggcaactgacaaacagccttatagcaagctcccaggtgtctctcttctgaaaccactgaaaggggtagatcctaacttaatcaacaacctggaaacattctttgaattggattatcccaaatatgaagtgctcctttgtgtacaagatcatgatgatccagccattgatgtatgtaagaagcttcttggaaaatatccaaatgttgatgctagattgtttataggtggcaaaaaagttggcattaatcctaaaattaataatttaatgccaggatatgaagttgcaaagtatgatcttatatggatttgtgatagtggaataagagtaattccagatacgcttactgacatggtgaatcaaatgacagaaaaagtaggcttggttcacgggctgccttacgtagcagacagacagggctttgctgccaccttagagcaggtatattttggaacttcacatccaagatactatatctctgccaatgtaactggtttcaaatgtgtgacaggaatgtcttgtttaatgagaaaagatgtgttggatcaagcaggaggacttatagcttttgctcagtacattgccgaagattactttatggccaaagcgatagctgaccgaggttggaggtttgcaatgtccactcaagttgcaatgcaaaactctggctcatattcaatttctcagtttcaatccagaatgatcaggtggaccaaactacgaattaacatgcttcctgctacaataatttgtgagccaatttcagaatgctttgttgccagtttaattattggatgggcagcccaccatgtgttcagatgggatattatggtatttttcatgtgtcattgcctggcatggtttatatttgactacattcaactcaggggtgtccagggtggcacactgtgtttttcaaaacttgattatgcagtcgcctggttcatccgcgaatccatgacaatatacatttttttgtctgcattatgggacccaactataagctggagaactggtcgctacagattacgctgtgggggtacagcagaggaaatcctagatgtataactacagctttgtgactgtatataaaggaaaaaagagaagtattataaattatgtttatataaatgcttttaaaaatctaccttctgtagttttatcacatgtatgttttggtatctgttctttaatttatttttgcatggcacttgcatctgtgaaaaaaaaaaaaaacacatctgtagtcttggccaaatgatacactttattttgtggaagtagagaatcaagagaattggttctaggaacctgggttcgttcatcattgttgtttatttttttaaaataaataaataaatatatatatagatgctctgcaacaaactctgtgacagtatccttcagtagagaaagtttcttacttgtgagtacactcttttagaacatttacgggtggcagcagctttgctttagagtttagaagataatagaaggaatgactattcatgtccaaagtgaatggttttgtgcagtgaacaacacatggcgaggtactaactgagaaactttttcatgctttatgcctacctcttgtagttgttgcagagcaaatataaattgtaataagatagctaggccttgcagaaacaaacagaaaaacttaaaaaaaaatgatataagagctggagtctagtatttatatgaatctgtgagagataatttttttggtctcactgcaatgaaccaaaagcggctgagtttggtttttaattgtagccatgtattgaaggcatctttttgaccaactcttgttggttctgtcttgaaccattgttaatcactgtgctgtaattagtatagctaaatcttttccttccttgctcctcccccagcccaccccgtcttcccttaacattttttcagggggggttgggagtggtttcattttagtgtgagtggatgttttgatagttgtaaggaaaaaatgcatttcagacacatttcacacatgagctattttcttacacagtatgtcttattgttaataagaatgtaatttcatgatacaagtatttaataccttttttaagtcagtaaatattctagccatcaataaattgcagtagagtattaagaggggacaggatgagtttactcttaaaattaatcagtatcaaactaagtctactgtgtgtgttttttttttttgttttttgtttttgtttgtttactattatagtaagtctagtcttatttaaattggatttttgtattcaagtttatatgacaaaatagactagatcagcgctggttgtaaatgcatgctgtacctcactgttttctccccatcatgccgctaaacttgctcgccaggttgtgcttaacttgtggtgaactggacttgattctgttttgttttgtttttttaaaaaaaaaaaacaaaaatgtaagggacagtgcataagccttttctttgttaagtagtggtgttttgctttttcttcaggattggatggcatgttgtccacaggaatgcctactgcagggctaacactggcaatatggcagctttaaattctgagttactacagtctccatgtcaaagcataatgtgtagcatatcagcaataactacatatttatagaaaacactttttcactttactgagccaattcatttaattgtcaaatgtctatatccatggtttaaatggcaacatatctataagtatgtatacacattatatttaagcttttctctgggccaaactgcttcatccttttttctttctttttttttttttttagccttatgatgaatttgtttgaagggcattttctttatgaacaaaggcttggatgcatattcctttctttctgtgaatgggtattattccctgaggaaagttgcacagtgaaaaccagtctggttgtgacccactacatgttttgtttttaatcactattacctgagttgaactttgctcaccatgtttgtacttgttggtctgtttaatgaagtttggttgatgccatcctttgcactgccgaagcgtaatcttgtgtattacttagctctctgctgatctcagtatggacagtgtaacaacaaaaccaaaatggctggacagacttcttgtgttttgtaaatataaactaggacagttctgtaggtttgttcagtgtgctagtggagtatttccttaatgtaaaacttcacttaacagagagattctttgtttagcaagcttggagtgatgataaaatggtaagaaataatataaatgttgaagaaagcatcacaacagaactataggagtctaaatttaataaatctttaaaaaaaaccagtgtctagaatatataccatgttttattatttaaaatcattgtcttaaatttttgttcaaaaaataaaaatttgaatacaatcaaaa

SEQ ID NO: 12 - protein, 394 aa, Homo sapiens
MALLDLALEGMAVFGFVLFLVLWLMHFMAIIYTRLHLNKKATDKQPYSKLPGVSLLKPLKGVDPNLINNLETFFELDYPKYEVLLCVQDHDDPAIDVCKKLLGKYPNVDARLFIGGKKVGINPKINNLMPGYEVAKYDLIWICDSGIRVIPDTLTDMVNQMTEKVGLVHGLPYVADRQGFAATLEQVYFGTSHPRYYISANVTGFKCVTGMSCLMRKDVLDQAGGLIAFAQYIAEDYFMAKAIADRGWRFAMSTQVAMQNSGSYSISQFQSRMIRWTKLRINMLPATIICEPISECFVASLIIGWAAHHVFRWDIMVFFMCHCLAWFIFDYIQLRGVQGGTLCFSKLDYAVAWFIRESMTIYIFLSALWDPTISWRTGRYRLRCGGTAEEILDV

SEQ ID NO: 13 - DNA, 21 bp, Artificial Sequence
  Artifically synthesized oligonucleotide
cttctaaccgaggtcgaaacg

SEQ ID NO: 14 - DNA, 22 bp, Artificial Sequence
  Artifically synthesized oligonucleotide
ggcattttggacaaagcgtcta

SEQ ID NO: 15 - DNA, 20 bp, Artificial Sequence
  Artifically synthesized oligonucleotide
aagcttcggctacagctcgg

SEQ ID NO: 16 - DNA, 20 bp, Artificial Sequence
  Artifically synthesized oligonucleotide
tcctaacttaatcaacaacc

SEQ ID NO: 17 - protein, 85 aa, Artificial Sequence
  Artifically truncated UGCG amino acid sequence
MALLDLALEGMAVFGFVLFLVLWLMHFMAIIYTRLHLNKKATDKQPYSKLPGVSLLKPLKGVDPNLIKLNPNLTWKHSLNWIIPK

(19) United States
(12) Patent Application Publication
(10) Pub. No.: US 2022/0257604 A1
(43) Pub. Date: Aug. 18, 2022

(54) COMPOSITIONS AND METHODS FOR MODULATING VIRAL INFECTIONS BY REGULATING GLUCOSYLCERAMIDES

(71) Applicant: University of Virginia Patent Foundation, Charlottesville, VA (US)

(72) Inventors: Mark Kester, Afton (US); Kelly C. Drews, Durham (US)

(73) Assignee: University of Virginia Patent Foundation, Charlottesville, VA (US)

(21) Appl. No.: 17/437,010

(22) PCT Filed: Mar. 9, 2020
(86) PCT No.: PCT/US2020/021699

§ 371 Date: Sep. 7, 2021

Related U.S. Application Data
(60) Provisional application No. 62/815,469, filed on Mar. 8, 2019.

(51) Int. Cl. A61K 31/5375; C07K 16/40; A61K 31/336; A61K 31/40; A61K 31/437; A61K 31/445; A61K 31/133; A61K 31/4025; A61P 31/16

(52) U.S. Cl. CPC A61K 31/5375; C07K 16/40; A61K 31/336; A61K 31/40; A61K 39/00; A61K 31/445; A61K 31/133; A61K 31/4025; A61P 31/16; A61K 31/437

(57) ABSTRACT

Provided are methods and compositions for inhibiting viral infections. In some embodiments, the methods include administering to a subject infected with and/or at risk for infection with a virus a composition having a glucosylceramidase inhibitor, a glucosylceramide synthase inhibitor, or any combination thereof via a route and in an amount effective for treating and/or inhibiting the viral infection in the subject. Also provided are methods for inhibiting viral infections of cells, methods for inhibiting endosomal fusion of viruses in cells, and compositions for use in treating and/or inhibiting viral infections of subjects and/or cells.

### CROSS REFERENCE TO RELATED APPLICATION

The presently disclosed subject matter claims the benefit of U.S. Provisional Patent Application Ser. No. 62/815,469, filed Mar. 8, 2019; the disclosure of which is incorporated herein by reference in its entirety.

### GOVERNMENT INTEREST

This invention was made with government support under Grant Nos. AI114776 and GN008715-16 awarded by The National Institutes of Health. The government has certain rights in the invention.

### BACKGROUND

Between three and five million people are infected with influenza A virus (IAV) worldwide each year, with one quarter to half a million cases resulting in death. While therapies against influenza exist, they are often administered too late to provide patient relief. Vaccines against the virus are produced each year, but often provide limited coverage against isolates arising from antigenic shift, such as occurred during the 2009 H1N1 pandemic, which is estimated to have killed up to 575,000 people (Dawood et al., 2012).

IAV is a negative sense RNA virus belonging to the family Orthomyxoviridae and is an enveloped virus that derives its lipid-bilayer membrane as the virus buds through the host plasma membrane during virus assembly. To infect a cell, influenza employs its hemagglutinin (HA) protein to bind to sialic acid moieties on the target cell surface and then is taken into the cell by endocytosis (Imai & Kawaoka, 2012). As the virus travels along the endocytic pathway, the acid environment prevailing in endosomes prompts conformational changes in HA, leading to viral membrane fusion with a late endosomal membrane (at pH ˜5.0 to 5.7 depending on the strain) and subsequent genome release into the cytoplasm to initiate replication (Sieczkarski & Whittaker, 2003; White et al., 2008; Li et al., 2014; Batishchev et al., 2016; White & Whittaker, 2016). Hence, proper endosomal trafficking and pH are crucial to the influenza life cycle (Ochiai et al., 1995; Gillespie et al., 2013; Yeganeh et al., 2015).

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides methods for treating and/or inhibiting viral infections in subjects. In some embodiments, the methods comprise administering to a subject infected with and/or at risk for infection with a virus a composition comprising a glucosylceramidase inhibitor, a glucosylceramide synthase inhibitor, or any combination thereof via a route and in an amount effective for treating and/or inhibiting the viral infection in the subject. In some embodiments, the viral infection is from a virus selected from the group consisting of an influenza A virus, an influenza B virus, an influenza C virus, a vesicular stomatitis virus (VSV), an Ebola virus (EBOV), a measles virus, a coronavirus, optionally COVID-19, and any combination thereof. In some embodiments, the glucosylceramidase inhibitor is a small molecule, an anti-glucosylceramidase antibody, an inhibitory nucleic acid that targets a glucosylceramidase gene product, or any combination thereof. In some embodiments, the glucosylceramidase inhibitor is selected from the group consisting of conduritol b epoxide (i.e., a mixture of 1-L-1,2-anhydro-myo-inositol and 1-D-1,2-anhydro-myo-inositol), D-threo-1-phenyl-2-palmitoylamino-3-pyrrolidino-1-propanol (P4; U.S. Pat. No. 8,168,587), castanospermine ((1S,6S,7R,8R,8aR)-octahydroindolizine-1,6,7,8-tetrol), isofagomine (5R-(hydroxymethyl)-3R,4R-piperidinediol, mono 2S,3S-dihydroxybutanedioate), valienamine, ((1S,2S,3R,6S)-6-Amino-4-(hydroxymethyl)cyclohex-4-ene-1,2,3-triol), validamine, ((1R,2S,3S,4S,6R)-4-amino-6-(hydroxymethyl)cyclohexane-1,2,3-triol), derivatives and salts thereof, and/or combinations thereof. In some embodiments, the glucosylceramide synthase inhibitor is a small molecule, an anti-glucosylceramide synthase antibody, an inhibitory nucleic acid that targets a glucosylceramide synthase gene product, or any combination thereof. In some embodiments, the glucosylceramide synthase inhibitor is selected from the group consisting of an iminosugar, N-butyl-deoxynojirimycin (miglustat), [(3S)-1-azabicyclo[2.2.2]octan-3-yl] N-[2-[2-(4-fluorophenyl)-1,3-thiazol-4-yl]propan-2-yl]carbamate (ibiglustat), N-[(1R,2R)-1-(2,3-Dihydro-1,4-benzodioxin-6-yl)-1-hydroxy-3-(1-pyrrolidinyl)-2-propanyl]octanamide (eliglustat), 1-phenyl-2-palmitoylamino-3-morpholino-1-propanol, and derivatives and/or combinations thereof. In some embodiments, the subject is a human.

In some embodiments, the presently disclosed subject matter also provides methods for inhibiting infection of a cell with a virus. In some embodiments, the methods comprise contacting the cell with a composition comprising a glucosylceramidase inhibitor, a glucosylceramide synthase inhibitor, or any combination thereof in an amount sufficient for inhibiting infection of the cell with the virus. In some embodiments, the virus selected from the group consisting of an influenza A virus, an influenza B virus, an influenza C virus, a vesicular stomatitis virus (VSV), an Ebola virus (EBOV), a measles virus, a coronavirus, optionally COVID-19, and any combination thereof. In some embodiments, the glucosylceramidase inhibitor is a small molecule, an anti-glucosylceramidase antibody, an inhibitory nucleic acid that targets a glucosylceramidase gene product, or any combination thereof. In some embodiments, the glucosylceramidase inhibitor is selected from the group consisting of conduritol b epoxide (i.e., a mixture of 1-L-1,2-anhydro-myo-inositol and 1-D-1,2-anhydro-myo-inositol), D-threo-1-phenyl-2-palmitoylamino-3-pyrrolidino-1-propanol (P4; U.S. Pat. No. 8,168,587), castanospermine ((1S,6S,7R,8R,8aR)-octahydroindolizine-1,6,7,8-tetrol), isofagomine (5R-(hydroxymethyl)-3R,4R-piperidinediol, mono 2S,3S-dihydroxybutanedioate), valienamine, ((1S,2S,3R,6S)-6-Amino-4-(hydroxymethyl)cyclohex-4-ene-1,2,3-triol), validamine, ((1R,2S,3S,4S,6R)-4-amino-6-(hydroxymethyl)cyclohexane-1,2,3-triol), derivatives and salts thereof, and/or combinations thereof. In some embodiments, the glucosylceramide synthase inhibitor is a small molecule, an anti-glucosylceramide synthase antibody, an inhibitory nucleic acid that targets a glucosylceramide synthase gene product, or any combination thereof. In some embodiments, the glucosylceramide synthase inhibitor is selected from the group consisting of an iminosugar, N-butyl-deoxynojirimycin (miglustat), [(3S)-1-azabicyclo[2.2.2]octan-3-yl] N-[2-[2-(4-fluorophenyl)-1,3-thiazol-4-yl]propan-2-yl]carbamate (ibiglustat), N-[(1R,2R)-1-(2,3-Dihydro-1,4-benzodioxin-6-yl)-1-hydroxy-3-(1-pyrrolidinyl)-2-propanyl]octanamide (eliglustat), 1-phenyl-2-palmitoylamino-3-morpholino-1-propanol, and derivatives and/or combinations thereof. In some embodiments, the cell is a human cell, which in some embodiments is present within a subject.

In some embodiments, the presently disclosed subject matter provides methods for inhibiting endosomal fusion of a virus in a cell. In some embodiments, the methods comprise contacting a cell with a glucosylceramidase inhibitor, a glucosylceramide synthase inhibitor, or any combination thereof, wherein an amount of the glucosylceramidase inhibitor, the glucosylceramide synthase inhibitor, or the combination thereof is effective for inhibiting endosomal fusion of the virus in the cell. In some embodiments, the virus selected from the group consisting of an influenza A virus, an influenza B virus, an influenza C virus, a vesicular stomatitis virus (VSV), an Ebola virus (EBOV), a measles virus, a coronavirus, optionally COVID-19, and any combination thereof. In some embodiments, the glucosylceramidase inhibitor is selected from the group consisting of conduritol b epoxide (i.e., a mixture of 1-L-1,2-anhydro-myo-inositol and 1-D-1,2-anhydro-myo-inositol), D-threo-1-phenyl-2-palmitoylamino-3-pyrrolidino-1-propanol (P4; U.S. Pat. No. 8,168,587), castanospermine ((1S,6S,7R,8R,8aR)-octahydroindolizine-1,6,7,8-tetrol), isofagomine (5R-(hydroxymethyl)-3R,4R-piperidinediol, mono 2S,3S-dihydroxybutanedioate), valienamine, ((1S,2S,3R,6S)-6-Amino-4-(hydroxymethyl)cyclohex-4-ene-1,2,3-triol), validamine, ((1R,2S,3S,4S,6R)-4-amino-6-(hydroxymethyl)cyclohexane-1,2,3-triol), derivatives and salts thereof, and/or combinations thereof; and/or the glucosylceramide synthase inhibitor is a small molecule, an anti-glucosylceramide synthase antibody, an inhibitory nucleic acid that targets a glucosylceramide synthase gene product, derivatives and salts thereof, and/or combinations thereof, optionally, wherein the glucosylceramide synthase inhibitor is selected from the group consisting of an iminosugar, N-butyl-deoxynojirimycin (miglustat), [(3S)-1-azabicyclo[2.2.2]octan-3-yl] N-[2-[2-(4-fluorophenyl)-1,3-thiazol-4-yl]propan-2-yl]carbamate (ibiglustat), N-[(1R,2R)-1-(2,3-Dihydro-1,4-benzodioxin-6-yl)-1-hydroxy-3-(1-pyrrolidinyl)-2-propanyl]octanamide (eliglustat), 1-phenyl-2-palmitoylamino-3-morpholino-1-propanol, and derivatives and/or combinations thereof. In some embodiments, the cell is a human cell, which in some embodiments is present within a subject.

In some embodiments, the presently disclosed subject matter provides compositions for use in treating and/or inhibiting viral infections in a subject and/or for inhibiting infection of a cell with a virus. In some embodiments, the compositions comprise a glucosylceramidase inhibitor, a glucosylceramide synthase inhibitor, or any combination thereof, wherein the amount of the glucosylceramidase inhibitor, the glucosylceramide synthase inhibitor, or the combination thereof is effective for treating and/or inhibiting the viral infection in a subject and/or for inhibiting infection of the cell with the virus. In some embodiments, the virus selected from the group consisting of an influenza A virus, an influenza B virus, an influenza C virus, a vesicular stomatitis virus (VSV), an Ebola virus (EBOV), a measles virus, a coronavirus, optionally COVID-19, and any combination thereof. In some embodiments, the glucosylceramidase inhibitor is selected from the group consisting of conduritol b epoxide (i.e., a mixture of 1-L-1,2-anhydro-myo-inositol and 1-D-1,2-anhydro-myo-inositol), D-threo-1-phenyl-2-palmitoylamino-3-pyrrolidino-1-propanol (P4; U.S. Pat. No. 8,168,587), castanospermine ((1S,6S,7R,8R,8aR)-octahydroindolizine-1,6,7,8-tetrol), isofagomine (5R-(hydroxymethyl)-3R,4R-piperidinediol, mono 2S,3S-dihydroxybutanedioate), valienamine, ((1S,2S,3R,6S)-6-Amino-4-(hydroxymethyl)cyclohex-4-ene-1,2,3-triol), validamine, ((1R,2S,3S,4S,6R)-4-amino-6-(hydroxymethyl)cyclohexane-1,2,3-triol), derivatives and salts thereof, and/or combinations thereof; and/or the glucosylceramide synthase inhibitor is a small molecule, an anti-glucosylceramide synthase antibody, an inhibitory nucleic acid that targets a glucosylceramide synthase gene product, derivatives and salts thereof, and/or combinations thereof, optionally, wherein the glucosylceramide synthase inhibitor is selected from the group consisting of an iminosugar, N-butyl-deoxynojirimycin (miglustat), [(3S)-1-azabicyclo[2.2.2]octan-3-yl] N-[2-[2-(4-fluorophenyl)-1,3-thiazol-4-yl]propan-2-yl]carbamate (ibiglustat), N-[(1R,2R)-1-(2,3-Dihydro-1,4-benzodioxin-6-yl)-1-hydroxy-3-(1-pyrrolidinyl)-2-propanyl]octanamide (eliglustat), 1-phenyl-2-palmitoylamino-3-morpholino-1-propanol, and derivatives and/or combinations thereof. In some embodiments, the subject is a human and/or the cell is a human cell. In some embodiments, the human cell is present within a subject.

In some embodiments, the presently disclosed subject matter also provides pharmaceutical compositions for treating and/or inhibiting a viral infection in a cell and/or in a subject and/or for inhibiting endosomal fusion of a viruses in a cell. In some embodiments, the pharmaceutical compositions comprise, consist essentially of, or consist of an effective amount of one or more glucosylceramidase inhibitors, one or more glucosylceramide synthase inhibitors, or any combination thereof.

In some embodiments, the presently disclosed subject matter also provides compositions for preparation of a medicament for treating and/or inhibiting a viral infection in a cell and/or in a subject and/or for inhibiting endosomal fusion of a viruses in a cell. In some embodiments, the compositions comprise, consist essentially of, or consist of an effective amount of one or more glucosylceramidase inhibitors, one or more glucosylceramide synthase inhibitors, or any combination thereof.

Accordingly, it is an object of the presently disclosed subject matter to provide compositions and methods for inhibiting viral infections.

This and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, Figures, and EXAMPLES. Additionally, various aspects and embodiments of the presently disclosed subject matter are described in further detail below.

### BRIEF DESCRIPTION OF THE FIGURES

FIG. 1 is a schematic diagram of the biochemical pathways involved in ceramide and glucocertamide biosynthesis and metabolism. The sphingolipid pathway involves numerous enzymes and lipids, most of which shuttle through ceramide as the pathway hub. Previous studies showed that deficiencies in sphingomyelin synthase as well as inhibition of serine palmitoyltransferase or sphingosine kinase led to decreased influenza infection. However, the glycosphingolipid arm of the sphingolipid pathway has not yet been studied in the context of influenza. Disclosed herein in some embodiments are experiments designed to investigate the biological activities of glucosylceramidase to determine the effects of glucosylceramide metabolism on influenza infections.

FIGS. 2A-2E summarize the results of experiments showing that glucosylceramidase activity influences the efficiency of influenza infection. HEK 293 and A549 cells were transfected with plasmids encoding Cas9-sgRNA targeting GBA and a plasmid containing GFP. Single cell colonies were selected for successful transfection as measured by GFP expression and expanded. The gRNA used to target GBA was 5′-AAGCTTCGGCTACAGCTCGG-3′ (SEQ ID NO: 15). FIG. 2A is a western blot confirming complete loss of GBA protein expression in lysates of both HEK 293 and A549 cell colonies. FIGS. 2B and 2C are bar graphs showing the results of mass spectrometry analyses of lipids extracted from HEK 293 cells and A549 cells, respectively. Consistent with KO status, total GlcCer levels were raised in both HEK 293 and A549 GBA knockouts. Data represent the mean values of six biological replicates±SE. Loss of GBA activity was confirmed using a direct enzyme assay. For FIGS. 2D and 2E, cells in triplicate samples were infected at 4° C. with PR8 influenza encoding an NS1-GFP chimeric protein and then incubated for ˜18 hours at 37° C. The cells were then harvested, fixed, and analyzed for GFP expression by flow cytometry. In both HEK 293 (FIG. 2D) and A549 (FIG. 2E) cell lines GBA knockout resulted in decreased influenza infection compared to WT as measured by NS1-GFP expression. Data represent the mean±SE, n=6 experiments. **p<0.01 using a Mann-Whitney non-parametric test. The growth rates of WT and GBA KO cells were similar over the duration studied (up to 4 days).

FIG. 3 is a schematic representation of biochemical pathways for lipid synthesis. De novo synthesis of ceramide is indicated.

FIGS. 4A and 4B are bar graphs showing that influenza M1 gene expression was time- and trypsin-dependent in GBA KO cells. WT (black bars) and GBA KO (gray bars) cells in triplicate samples were incubated at 4° C. with PR8 influenza and then incubated for 8 or 24 hours at 37° C. with or without trypsin in the medium. Samples were collected and mRNA extracted, cDNA generated, and relative gene expression analyzed by qPCR. FIG. 4A is a bar graph showing that GBA KO cells displayed a reduction in influenza M1 expression when incubated without trypsin, and therefore limited to one cycle of influenza infection. FIG. 4B is a bar graph showing that influenza M1 expression is reduced after 8 hours in GBA KO cells in the presence of trypsin, but after 24 hours the expression matches that of WT cells. n.s.: not significant. *: .<0.05.

FIGS. 5A-5C present the results of experiments showing that loss of GBA reduced influenza virus fusion in endosomes. Influenza was labeled with R18 (a red fluorescent dye) and DiOC18 (a green fluorescent dye) and then added to prechilled A549 cells at 4° C. for 15 minutes. Cells were then washed, incubated at 37° C. for 30 minutes, fixed, and imaged at 60× magnification. The number of green (indicating a fused virus) and red (indicating an unfused virus) particles were then analyzed using ImageJ particle analysis. WT cells pretreated with 100 nM bafilomycin, an endosome acidification inhibitor, for 1 hour served as a positive control. FIG. 5A shows the ratio of fused to unfused particles measured by automated counting of the number of green particles divided by the number of red particles on an image by image basis from two experiments (n=40 fields for each treatment). Each data point represents the total number of green particles divided by the total number of red particles for 1 image field, and the bars indicate mean values±SD. (see Materials and Methods section below for details). FIGS. 5B and 5C are representative fluorescent micrograph images. Cell outlines were marked by visual examination. *p<0.05, ***p<0.001 using 665 a Mann-Whitney non-parametric test.

FIGS. 6A-6E are a series of bar graphs presenting the results of experiments showing that loss of GBA reduced entry mediated by the glycoproteins of other endosome-entering enveloped viruses, with minimal effects on entry mediated by the glycoproteins of measles virus, a plasma-membrane entering virus. Influenza virus like particles (VLPs) bearing the VSV-G, WSN HA/NA, or EBOV-GPΔ glycoproteins were generated on a βlaM1 backbone as described in the Materials and Methods section below. VLPs were added to prechilled HEK 293 cells (FIG. 6A) or A549 cells (FIG. 6B), and the complexes were centrifuged at 4° C. for 1 hour, incubated for 3 hours at 37° C., and then incubated for 1 hour at room temperature in the dark in the presence of a fluorescent β-lactamase substrate. Cells were washed and the following day were harvested, fixed, and analyzed for β-lactamase activity via flow cytometry. See Materials and Methods section below for details. Data represent the mean±SE, n=6 experiments. VSV pseudoviruses bearing the Measles-F and HN, VSV-G, or EBOV-GPΔ glycoproteins and encoding GFP were generated and bound to prechilled HEK 293 cells (FIG. 6C) or A549 cells (FIG. 6D) by centrifugation at 4° C. for 1 hour. The pseudovirus-cell complexes were then incubated at 37° C. for 24 hours, after which they were lifted, fixed, and analyzed for GFP expression via flow cytometry. Data represent the mean±SE, n=6 experiments. *p<0.05, **p<0.01 using a Mann-Whitney non-parametric test. FIG. 6E is a bar graph showing relative infection of WT (black bar) and GBA KO HEK 293 (gray bar) cells were infected with EBOV trVLPs for 24 hr at 37° C. and infection was assayed as described in the Materials and Methods section below. Data represent the mean±SE, n=5. *p<0.05 using a Mann-Whitney non-parametric test.

FIGS. 7A and 7B are a series of bar graph presenting the results of experiments showing that loss of GBA did not detectably alter lysosomal pH. HEK 293 cells (FIG. 7A) or A549 cells (FIG. 7B) were incubated with FITC-dextran for 72 hours followed by a 2 hour pulse in medium without FITC-dextran. Cells were pretreated with bafilomycin (Baf) or NH₄Cl where indicated for 1 hour at 37° C. to serve as positive controls. Following the dextran-free pulse, cells were analyzed by flow cytometry. Samples were compared to a standard curve of pH controls to determine lysosomal pH, as described in the Materials and Methods section below. Data represent the mean±SE, n=5 experiments. n.s.: not significant; **p<0.01.

FIGS. 8A-8D present the results of experiments showing that trafficking of influenza to late endosomes was impaired in GBA KO cells. A549 cells were transfected with Lamp1-GFP one day prior to experiments. WT cells were pretreated with 40 μM nocodazole (as a positive control) for 1 hour where indicated. Influenza (PR8) was labeled with R18 and then added to prechilled cells at an MOI ˜10 at 4° C. for 15 minutes. Cells were washed, incubated at 37° C. for 40 minutes, fixed and imaged at 100× magnification. FIG. 8A is a graph of average Manders colocalization coefficients of influenza with Lamp1 (±SD) from 2 experiments (n=100 fields in each experiment). Each data point represents the Manders colocalization coefficient for 1 image field. FIGS. 8B and 8C are representative fluorescence micrographs of wild type and GBA KO cells, respectively, infected with R18-labeled influenza. Numbered white boxes are enlarged to the right of each panel. Examples are of colocalized particles in the WT cells and examples of non-colocalized particles in the knockout cells. FIG. 8D is a graph of total number of influenza virions in each image analyzed in FIG. 8A. n.s.: not significant. ****p<0.0001 based on a Mann-Whitney non-parametric test.

FIGS. 9A-9D present the results of experiments showing that trafficking of EGF to late endosomes was impaired in GBA KO cells. A549 cells were transfected with Lamp1-GFP and pretreated with nocodozaole as in FIGS. 6A-6E. EGF-555 (100 ng/mL) was added to cells prechilled to 4° C. for 15 minutes. Cells were then washed, incubated at 37° C. for 40 minutes, fixed, and imaged. FIG. 9A is a graph of average Manders colocalization coefficients of EGF with Lamp1 (±SD) from 2 experiments (n=100 fields in each experiment). Each data point represents the Manders colocalization coefficient for 1 image field. FIGS. 9B and 9C are representative fluorescence micrographs of wild type (WT) and GBA KO cells, respectively, incubated with EGF-555. White boxes are enlarged to allow better qualitative visualization. Examples of colocalized particles pictured in the WT cells (FIG. 9B) and examples of non-colocalized particles pictured in the knockout cells (FIG. 9C). FIG. 9D is a graph of the total number of EGF particles in each image analyzed in FIG. 9A. **p<0.01, ****p<0.0001 using a Mann-Whitney non parametric test.

FIGS. 10A and 10B present the results of experiments showing that EGFR degradation was impaired in GBA KO Cells. EGF (50 ng/mL) was added to cells and the cells incubated at 37° C. At the indicated times, cell lysates were prepared and subjected to SDS-PAGE and Western blotting for EGFR. FIG. 10A is a representative Western blot. FIG. 10B is a graph of quantitation of Western blots, which indicated a significant difference in EGFR remaining at 2 hours post addition of EGF in GBA KO cells (triangles) as compared to WT (squares). Values are normalized to the intensity of GAPDH and presented as a percentage of EGFR remaining as compared to cells without EGF stimulation. Data represent the mean±SE, n=6 experiments. *p<0.05 using a Mann-Whitney non-parametric test.

FIGS. 11A-11D present the results of experiments showing that loss of GBA upregulated cathepsin B activity. FIG. 11A is a bar graph of WT and GBA KO A549 cells that were lysed and analyzed for cathepsin B activity following incubation with a cathepsin B-specific substrate for 1 hour at 37° C., as described in the Materials and Methods section herein below. Data represent the mean±SE, n=5 experiments. FIG. 11B is a bar graph of cathepsin B activity in vivo in wild type A549 cells as determined using the Magic Red-(RR)2 cathepsin activity assay as per the manufacturers instructions. Data represent the mean±SE, n=5 experiments. FIG. 11C shows the results of cathepsin B probed by Western Blot and found to be undetectable in A549 WT samples but readily detectable in GBA KO cells. Loading 2.5× the amount of lysate resulted in CatB detection in the WT cells. FIG. 11D is a bar graph of WT and GBA KO cells that were lysed and processed as in FIG. 11A, but incubated with a cathepsin L-specific substrate and analyzed for cathepsin L activity as described in the Materials and Methods section below. *p<0.05, **p<0.01 using a Mann-Whitney non-parametric test.

FIG. 12 is a schematic diagram of the biochemical pathways involved in ceramide and glucocertamide biosynthesis and metabolism with particular reference to the involvement of UGCG. Sphingolipids and influenza virus infections. Several studies have demonstrated that inhibition of distinct enzymes in the sphingolipid pathway results in alterations to influenza virus infection levels. Pharmacological inhibition of sphingomyelin synthesis (through serine palmitoyltransferase) and sphingosine kinase, as well as genetic ablation of sphingomyelin synthase and glucosylceramidase (shown by “X”s) led to decreased influenza virus infection (Seo et al., 2010; Seo et al., 2013; Tafesse et al., 2013; Drews et al., 2019). Conversely, reductions in ceramide synthesis through inhibition of ceramide synthase led to an increase in influenza virus replication (Soudani et al., 2019). The role of UGCG (identified by the box) in influenza virus infection by using the pharmacological inhibitor PPMP as well as by knocking out the gene encoding for UGCG enzyme expression in two cell lines.

FIGS. 13A-13E are a series of bar graphs showing the results of CRISPR/Cas9-mediated knockout of glucosylceramide synthase. FIG. 13A is a bar graph of HEK 293 cells pretreated with 20 μM PPMP for 48 hours or 100 nM bafilomycin for 1 hour and then infected with PR8 influenza virus encoding an NS1-GFP chimeric protein in the presence of the indicated drug, for 18-24 hours (selected time points chosen after optimization). Cells were then lifted, fixed, and analyzed by flow cytometry for GFP expression. PPMP-treated samples exhibited a 50% reduction in GFP signal compared to WT, indicating a role for UGCG in influenza virus infection. Data represent the mean values of 4 biological replicates (each performed in triplicate)±SE. HEK 293 and A549 cells were transfected with plasmids encoding GFP as well as Cas9-sgRNA targeting UGCG (5′-TCCTAACTTAATCAACAACC-3′; SEQ ID NO: 16). Cells were selected for GFP expression and single cell colonies were expanded and monitored for UGCG knockout as described in the Methods. Next, selected cell clones (see FIGS. 18A-18D) were assayed for UGCG activity by incubating cells with 5 μM C6-ceramide nanoliposome for 4 hours. Cells containing functional UGCG are able to convert C6-ceramide to C6-GlcCer, as seen in WT samples. HEK 293 (FIG. 13B) and A549 UGCG KO (FIG. 13C) cells displayed no C6-GlcCer, indicating a complete loss of UGCG activity. Lipids from WT and HEK 293 KO (FIG. 13D) or A459 KO (FIG. 13E) cells were analyzed by mass spectrometry. In agreement with the measured enzyme activity (FIGS. 13B and 13C), levels of total basal endogenous GlcCer were significantly reduced in both HEK 293 and A549 KO cells as compared to WT. Data represent the mean±SE (n=6 samples). **p<0.01 using a Mann-Whitney non-parametric test.

FIGS. 14A-14C present the results of experiments showing that A549 UGCG KO cells exhibit haploinsufficiency. FIG. 14A shows that relative loss of UGCG expression was confirmed in HEK 293 cells by western blot analysis. In comparison, A549 UGCG KO cells (based on DNA analysis; see the Materials and Methods section below) displayed only a reduced level (but not an absence) of UGCG protein on Western blots. Since no UGCG activity was detected in these cells (see FIGS. 13C and 13E), they are functionally null for UGCG and therefore haploinsufficient. Next generation sequencing was employed to determine the exact genetic alterations that had occurred in the A549 UGCG KO cells. It was determined that those cells displayed a heterozygous mutation, with one allele altered by the CRISPR/Cas9 activity to contain a frameshift resulting in a premature stop codon, while the other allele remained unaltered. A comparison of these amino acid sequences is presented in FIG. 14C, in which the wild type (WT) amino acid sequence (SEQ ID NO: 12) is presented above the UGCG KO sequence (SEQ ID NO: 17). These findings suggested that the induced mutation (stop codon) resulted in haploinsufficiency, as the functional activity of UGCG was completely lost in A549 KO cells (see FIGS. 13B-13E).

FIGS. 15A and 15B are bar graphs presenting the results of experiments showing that glucosylceramide synthase regulated influenza virus reinfection. Cells were infected with influenza virus as in FIG. 13A, and analyzed 18-24 hours later by flow cytometry. In FIG. 15A, HEK 293 UGCG KO cells exhibited an ˜40% reduction in influenza virus infection as compared to WT. In FIG. 15B, A549 UGCG KO cells exhibited ˜70% reduction in influenza virus infection as compared to WT. (mean±SE; n=6). **p<0.01 using a Mann-Whitney non-parametric test.

FIGS. 16A and 16B are bar graphs presenting the results of experiments showing that UGCG maintained optimal entry of VLPs bearing the glycoproteins of VSV, WSN influenza virus and EBOV. VLPs were generated on an influenza virus βlaM1 backbone with the indicated viral glycoprotein. VLPs were added to prechilled cells which were then centrifuged at 4° C. for 1 hour. Next the cells were incubated for 3 hours at 37° C., and then for 1 hour at room temperature in the presence of the βlaM substrate CCF2. Cells were washed, stored in the dark at room temperature, and (the following day) harvested, fixed, and analyzed for β-lactamase activity via flow cytometry. Entry by VLPs bearing VSV G was reduced in HEK 293 UGCG KO cells (FIG. 16A), but unaffected in A549 KO cells (FIG. 16B). WSN influenza virus glycoprotein-mediated entry was reduced in both KO cell lines, consistent with the findings in FIGS. 15A and 15B. Entry mediated by the EBOV glycoprotein was reduced in both 293 and A549 UGCG KO cells, and to a greater extent than seen with VLPs bearing the glycoproteins from WSN or VSV (mean±SE; n=6). **p<0.01 using a Mann-Whitney non-parametric test.

FIGS. 17A and 17B are bar graphs presenting the results of experiments showing the effects of loss of UGCG on infections by VSV pseudoviruses bearing the glycoproteins of measles virus, VSV, and EBOV. Pseudoviruses were generated using a VSV helper virus encoding GFP and the indicated viral glycoprotein(s). Pseudoviruses were then adhered to prechilled cells assisted by centrifugation at 4° C. for 1 hour. The cells were then washed, incubated at 37° C. and, the following day, harvested, fixed, and analyzed for GFP expression by flow cytometry. Infection by VSV pseudoviruses bearing the measles virus H and F proteins was unaffected in HEK 293 UGCG KO cells, and increased in the A549 UGCG KO cells. Infection by VSV pseudoviruses bearing the VSV glycoprotein was decreased in HEK 293 UGCG KO cells (FIG. 17A) but unaffected in A549 UGCG KO cells (FIG. 17B), consistent with the findings in FIGS. 16A and 16B. Infection by VSV pseudoviruses bearing the EBOV glycoprotein was decreased in both UGCG KO lines tested (mean±SE; n=6), also consistent with the results in FIGS. 16A and 16B. **p<0.01 using a Mann-Whitney non-parametric test.

FIGS. 18A-18D are bar graphs presenting the results of experiments showing screening and analysis of putative UGCG KO clones. Putative KO clones from both HEK293 (FIG. 18A) and A549 (FIG. 18B) cells were screened (n=1) by determining their ability to convert exogenously added C6 ceramide to C6 GlcCer, which was assayed by lipid mass spectroscopy. The clones that exhibited the greatest reduction in UGCG activity were selected for further experiments: clone B29 for HEK293 and clone A11 for A549 cells. The chosen KO cell lines (HEK 293 Cells and A549 Cells in FIGS. 18C and 18D, respectively) were analyzed to determine the effect of knocking out UGCG on cell growth rates to ensure that any experimental findings were not due to underlying differences in cell growth. (n=1, performed in triplicate).

### DETAILED DESCRIPTION

### I. General Considerations

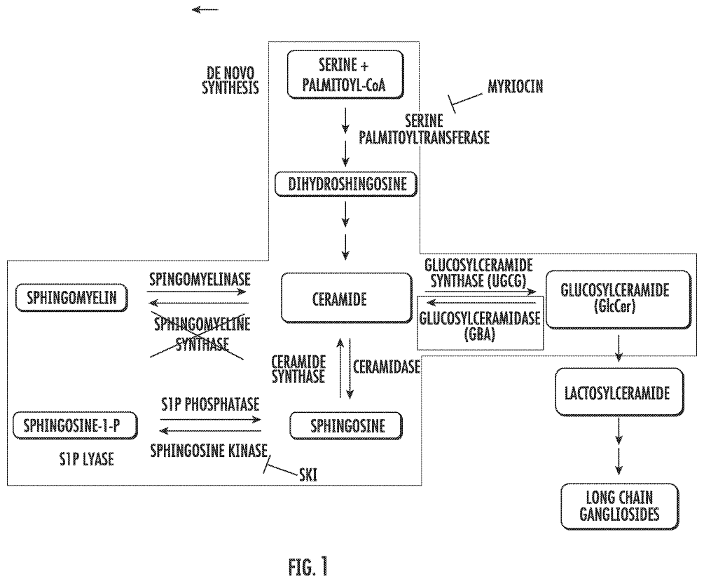

Influenza is an RNA virus encapsulated in a lipid bilayer derived from the host cell plasma membrane. The membrane of influenza contains sphingolipids, a class of bioactive signaling molecules broadly distributed in mammalian cells and integral to multiple cell functions (Gault et al., 2010). Sphingolipids have also been shown to play diverse roles in virus-host interactions (Schneider-Schaulies & Schneider-Schaulies, 2015), including promoting virus binding (Puri et al., 2004; Rawat et al., 2004; Grassmé et al., 2005; Dreschers et al., 2007), entry (Miller et al., 2012; Shivanna et al., 2015; Drake et al., 2017), replication (Weng et al., 2010; Konan & Sanchez-Felipe, 2014), and new particle release (Kanj et al., 2006). Several laboratories have explored the relationship between influenza virus and sphingolipids, notably sphingosine-1-phosphate (S1P) and sphingomyelin (see FIG. 1). Overexpression of S1P lyase lowered, while overexpression of sphingosine kinase increased, influenza infection in host cells (Seo et al., 2010). Moreover, influenza infection was shown to activate sphingosine kinase, generating sphingosine-1-phosphate, which was shown to increase viral RNA synthesis and nuclear export of influenza ribonucleoprotein complexes (Seo et al., 2013). Cells deficient in sphingomyelin synthase displayed reduced transport of the influenza glycoproteins (HA and neuraminidase) to the cell surface, and pharmacological reduction of sphingomyelin with myriocin led to decreased influenza infection (Tafesse et al., 2013).

At the hub of sphingolipid metabolism is ceramide, an apoptosis-inducing 93 molecule that can be modified at both its polar head group and carbohydrate chain to generate numerous sphingolipid species (Shaw et al., 2018; see also FIG. 1). Ceramide is converted to the glycosphingolipid glucosylceramide (GlcCer) by the addition of a glucose moiety catalyzed by the enzyme glucosylceramide synthase (UGCG), which is found primarily in the Golgi (Ishibashi et al., 2013). Conversely, catabolism of GlcCer to remove the glucose group is performed by glucosylceramidase (GBA), which is reported to be primarily found in lysosomes (Schulze et al., 2009). Mutations in GBA are well-studied genetic determinates of Gaucher disease, one of the most common lysosomal storage disorders (Rosenbloom & Weinreb, 2013; Stirnemann et al., 2017).

While GBA has been implicated in trafficking of membrane glycolipids along the endocytic pathway (Tani et al., 2010), there have not been studies on the role of GBA in endocytic cargo trafficking. Moreover, while several studies have focused on the conversion of sphingomyelin to ceramide in the context of viral infections (see FIG. 1; Jan et al., 2000; Grassmé et al., 2005; Finnegan et al., 2007; Tani et al., 2010; Miller et al., 2012; Shivanna et al., 2015), little attention has been focused on the glucosylceramide arm of sphingolipid metabolism mediated by GBA and UGCG (see FIG. 1). One recent study employing a haploid genetic screen revealed a role for UGCG in infections by specific bunyaviruses, but not by other viruses tested (Drake et al., 2017). However, it does not appear that the role of GBA has been investigated in any viral infection.

To examine whether GBA is required for other enveloped viruses, entry mediated by the glycoproteins of Ebola, influenza, vesicular stomatitis, and measles viruses were tested in GBA knockout cells. Entry inhibition was relatively robust for Ebola and influenza, modest for VSV, and mild for measles, suggesting a greater role for viruses that enter cells by fusing with late endosomes. As the virus studies suggested a general role for GBA along the endocytic pathway, it was determined that trafficking of epidermal growth factor to late endosomes, as well as degradation of its receptor, were impaired in GBA knockout cells.

As disclosed herein, the role of GBA in influenza entry and infection was investigated by genetically knocking it out using CRISPR/Cas9. It was determined that cells deleted for GBA displayed reduced influenza trafficking to late endosomes and consequent fusion, entry, and infection, suggesting that GBA, and by extension optimal levels of its substrate lipid, GlcCer, are critical for maintaining the influenza life cycle in host cells. Also provided is evidence that GBA is required for the entry of other viruses that enter cells by endocytosis as well as for the proper trafficking and disposition of normal cellular vesicular cargos destined for late endosomes including EGF and its receptor.

Collectively the data presented herein suggested that GBA is critically important for endocytic trafficking of viruses as well as cellular cargos including growth factor receptors. Modulation of glucosylceramide levels may therefore represent a novel accompaniment to strategies to antagonize ‘late penetrating’ viruses, including influenza.

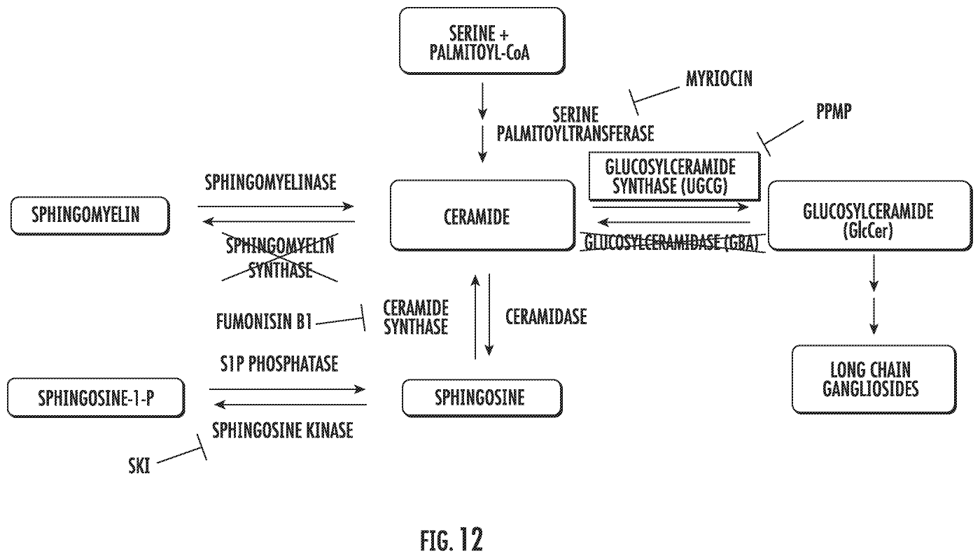

Previous studies have shown that influenza infection depends on cellular lipids including the sphingolipids sphingomyelin and sphingosine. Sphingolipid metabolism involves numerous enzymes and intermediary lipids, which predominantly shuttle through ceramide as a main hub (Shaw et al., 2018; see also FIG. 12). Upon addition of a glucose molecule by glucosylceramide synthase (UGCG), ceramide is converted into the glycosphingolipid glucosylceramide (GlcCer), a pro-survival signaling molecule and a precursor lipid for higher order gangliosides (Ichikawa et al., 1998). GlcCer is a relatively understudied sphingolipid in the context of viral infections, as most research focuses on sphingomyelin, the far more abundant sphingolipid found primarily in plasma membranes (see e.g., Jan et al., 2000; Grassmé et al., 2005; Finnegan et al., 2007; Tani et al., 2010; Miller et al., 2012; Shivanna et al., 2015).

Whether UGCG is involved in influenza virus infections is not known, however. As disclosed herein, the CRISPR/Cas9 system was employed to genetically knockout UGCG to thereby determine its role in influenza virus entry and infection. Disclosed herein are investigations into the role of a third sphingolipid, glucosylceramide, in influenza infection following CRISPR/Cas9-mediated knockout of its metabolizing enzyme glucosylceramidase (GBA). After confirming GBA knockout of HEK 293 and A549 cells by both western blotting and lipid mass spectrometry, diminished infection was observed in both KO cell lines by a PR8 (H1N1) GFP reporter virus. Additionally, reduction in infection correlated with impaired influenza trafficking to late endosomes, and hence fusion, and entry. As disclosed herein, diminished influenza virus infection was observed in HEK 293 and A549 UGCG knockout cells, which has been attributed to impaired viral entry. Entry mediated by the glycoproteins of other enveloped viruses that enter cells by endocytosis was also impaired in UGCG knockout cells, suggesting a broader role for UGCG in viral entry by endocytosis.

### II. Definitions

In describing and claiming the presently disclosed subject matter, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about”, as used herein, means approximately, in the region of, roughly, or around. When the term “about” is used in conjunction with a numerical range, it modifies that range by extending the boundaries above and below the numerical values set forth. For example, in some embodiments, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

As used herein, the phrase “biological sample” refers to a sample isolated from a subject (e.g., a biopsy, blood, serum, etc.) or from a cell or tissue from a subject (e.g., RNA and/or DNA and/or a protein or polypeptide isolated therefrom). Biological samples can be of any biological tissue or fluid or cells from any organism as well as cells cultured in vitro, such as cell lines and tissue culture cells. Frequently the sample will be a “clinical sample” which is a sample derived from a subject (i.e., a subject undergoing a diagnostic procedure and/or a treatment). Typical clinical samples include, but are not limited to cerebrospinal fluid, serum, plasma, blood, saliva, skin, muscle, olfactory tissue, lacrimal fluid, synovial fluid, nail tissue, hair, feces, urine, a tissue or cell type, and combinations thereof, tissue or fine needle biopsy samples, and cells therefrom. Biological samples can also include sections of tissues, such as frozen sections or formalin fixed sections taken for histological purposes.

As used herein, term “comprising”, which is synonymous with “including,” “containing”, or “characterized by”, is inclusive or open-ended and does not exclude additional, unrecited elements and/or method steps. “Comprising” is a term of art used in claim language which means that the named elements are present, but other elements can be added and still form a composition or method within the scope of the presently disclosed subject matter. By way of example and not limitation, a pharmaceutical composition comprising a particular active agent and a pharmaceutically acceptable carrier can also contain other components including, but not limited to other active agents, other carriers and excipients, and any other molecule that might be appropriate for inclusion in the pharmaceutical composition without any limitation.

As used herein, the phrase “consisting of” excludes any element, step, or ingredient that is not particularly recited in the claim. When the phrase “consists of” appears in a clause of the body of a claim, rather than immediately following the preamble, it limits only the element set forth in that clause; other elements are not excluded from the claim as a whole. By way of example and not limitation, a pharmaceutical composition consisting of an active agent and a pharmaceutically acceptable carrier contains no other components besides the particular active agent and the pharmaceutically acceptable carrier. It is understood that any molecule that is below a reasonable level of detection is considered to be absent.

As used herein, the phrase “consisting essentially of” limits the scope of a claim to the specified materials or steps, plus those that do not materially affect the basic and novel characteristic(s) of the claimed subject matter. By way of example and not limitation, a pharmaceutical composition consisting essentially of an active agent and a pharmaceutically acceptable carrier contains active agent and the pharmaceutically acceptable carrier, but can also include any additional elements that might be present but that do not materially affect the biological functions of the composition in vitro or in vivo.

With respect to the terms “comprising”, “consisting essentially of”, and “consisting of”, where one of these three terms is used herein, the presently disclosed and claimed subject matter encompasses the use of either of the other two terms. For example, “comprising” is a transitional term that is broader than both “consisting essentially of” and “consisting of”, and thus the term “comprising” implicitly encompasses both “consisting essentially of” and “consisting of”. Likewise, the transitional phrase “consisting essentially of” is broader than “consisting of”, and thus the phrase “consisting essentially of” implicitly encompasses “consisting of”.

The term “subject” as used herein refers to a member of any invertebrate or vertebrate species. Accordingly, the term “subject” is intended to encompass any member of the Kingdom Animalia including, but not limited to the phylum Chordata (i.e., members of Classes Osteichythyes (bony fish), Amphibia (amphibians), Reptilia (reptiles), Aves (birds), and Mammalia (mammals)), and all Orders and Families encompassed therein. In some embodiments, a subject is a human.

As used herein, the phrase “glucosylceramidase beta”, Gene Symbol GBA, refers to the glucosylceramidase beta locus, gene, and/or a gene product thereof, including but not limited to nucleic acids and polypeptides. In some embodiments, the GBA is a human GBA, the locus of which is present on human chromosome 1 and has a genomic DNA sequence exemplified by Accession No. NG_009783.1 of the GENBANK® biosequence database. Exemplary, non-limiting GBA gene product biosequences are as follows:

| | Nucleotide | Amino Acid |
|---|---|---|
| Description | Sequence | Sequence |
| Human, transcript variant 1 | NM_000157.4 | NP_00148.2 |
| | (SEQ ID NO: 1) | (SEQ ID NO: 2) |
| Human, transcript variant 2 | NM_001005741.3 | NP_001005741.1 |
| | (SEQ ID NO: 3) | (SEQ ID NO: 3) |
| Human, transcript variant 3 | NM_001005741.3 | NP_001005741.1 |
| | (SEQ ID NO: 5) | (SEQ ID NO: 6) |
| Human, transcript variant 4 | NM_001171811.2 | NP_001165282.1 |
| | (SEQ ID NO: 7) | (SEQ ID NO: 8) |
| Human, transcript variant 5 | NM_001171812.2 | NP_001165283.1 |
| | (SEQ ID NO: 9) | (SEQ ID NO: 10) |

As used herein, the phrase “UDP-glucose ceramide glucosyltransferase”, Gene Symbol UGCG, refers to the UDP-glucose ceramide glucosyltransferase locus, gene, and/or a gene product thereof, including but not limited to nucleic acids and polypeptides. In some embodiments, the UGCG is a human UGCG, the locus of which is present on human chromosome 9q31.3. Exemplary, non-limiting UGCG gene product biosequences are found in the GENBANK® biosequence database as Accession Nos. NM_003358.3 (nucleotide sequence) and NP_003349.1 (amino acid sequence).

It is noted that all genes, gene names, gene products, and other products disclosed herein are intended to correspond to orthologs or other similar products from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, any genes specifically mentioned herein and for which Accession Nos. for various exemplary gene products disclosed in the GENBANK® biosequence database, are intended to encompass homologous and variant genes and gene products from humans and other animals including, but not limited to other mammals. By way of example and not limitation, the GENBANK® biosequence database includes Accession Nos. NM_008094.6 and NM_001077411.3 corresponding to nucleotide sequences of mouse Gba gene products, and NM_001127639.1 corresponding to the nucleotide sequence of a rat Gba gene product, among others, and Accession No. NM_011673.3 corresponding to the nucleotide sequences of a mouse Ugcg gene product and NM_031795.2 corresponding to the nucleotide sequence of a rat Ugcg gene product, among others.

The methods of the presently disclosed subject matter are particularly useful for warm-blooded vertebrates. Thus, the presently disclosed subject matter concerns mammals and birds. More particularly contemplated is the isolation, manipulation, and use of stem cells from mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also contemplated is the isolation, manipulation, and use of stem cells from livestock, including but not limited to domesticated swine (pigs and hogs), ruminants, horses, poultry, and the like.

As used herein, the phrase “substantially” refers to a condition wherein in some embodiments no more than 50%, in some embodiments no more than 40%, in some embodiments no more than 30%, in some embodiments no more than 25%, in some embodiments no more than 20%, in some embodiments no more than 15%, in some embodiments no more than 10%, in some embodiments no more than 9%, in some embodiments no more than 8%, in some embodiments no more than 7%, in some embodiments no more than 6%, in some embodiments no more than 5%, in some embodiments no more than 4%, in some embodiments no more than 3%, in some embodiments no more than 2%, in some embodiments no more than 1%, and in some embodiments no more than 0% of the components of a collection of entities does not have a given characteristic.

The terms “additional therapeutically active compound” or “additional therapeutic agent”, as used in the context of the presently disclosed subject matter, refer to the use or administration of a compound for an additional therapeutic use for a particular injury, disease, or disorder being treated. Such a compound, for example, could include one being used to treat an unrelated disease or disorder, or a disease or disorder which is not responsive to the primary treatment for the injury, disease or disorder being treated. Diseases and disorders being treated by the additional therapeutically active agent include, for example, hypertension and diabetes. The additional compounds can also be used to treat symptoms associated with the injury, disease, or disorder, including, but not limited to, pain and inflammation.

The term “adult” as used herein, is meant to refer to any non-embryonic or non-juvenile subject.

As used herein, an “agonist” is a composition of matter which, when administered to a mammal such as a human, enhances or extends a biological activity attributable to the level or presence of a target compound or molecule of interest in the subject.

A disease or disorder is “alleviated” if the severity of a symptom of the disease, condition, or disorder, or the frequency with which such a symptom is experienced by a subject, or both, are reduced.

As used herein, amino acids are represented by the full name thereof, by the three letter code corresponding thereto, or by the one-letter code corresponding thereto, as indicated in Table 1:

TABLE 1
Amino Acid Codes and Functionally Equivalent Codons

| | 3-Letter | 1-Letter | Functionally Equivalent |
|---|---|---|---|
| Full Name | Code | Code | Codons |
| Aspartic Acid | Asp | D | GAC; GAU |
| Glutamic Acid | Glu | E | GAA; GAG |
| Lysine | Lys | K | AAA; AAG |
| Arginine | Arg | R | AGA; AGG; CGA; CGC; CGG; |
| | | | CGU |
| Histidine | His | H | CAC; CAU |
| Tyrosine | Tyr | Y | UAC; UAU |
| Cysteine | Cys | C | UGC; UGU |
| Asparagine | Asn | N | AAC; AAU |
| Glutamine | Gln | Q | CAA; CAG |
| Serine | Ser | S | ACG; AGU; UCA; UCC; UCG; |
| | | | UCU |
| Threonine | Thr | T | ACA; ACC; ACG; ACU |
| Glycine | Gly | G | GGA; GGC; GGG; GGU |
| Alanine | Ala | A | GCA; GCC; GCG; GCU |
| Valine | Val | V | GUA; GUC; GUG; GUU |
| Leucine | Leu | L | UUA; UUG; CUA; CUC; CUG; |
| | | | CUU |
| Isoleucine | Ile | I | AUA; AUC; AUU |
| Methionine | Met | M | AUG |
| Proline | Pro | P | CCA; CCC; CCG; CCU |
| Phenylalanine | Phe | F | UUC; UUU |
| Tryptophan | Trp | W | UGG |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter.

The term “amino acid” is used interchangeably with “amino acid residue,” and can refer to a free amino acid or to an amino acid residue of a peptide. It will be apparent from the context in which the term is used whether it refers to a free amino acid or a residue of a peptide.

Amino acids can be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe peptide compounds as disclosed herein follows the conventional practice wherein the amino group is presented to the left and the carboxy group to the right of each amino acid residue. In the formulae representing selected specific embodiments of the presently disclosed subject matter, the amino-and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

An “antagonist” is a composition of matter which when administered to a mammal such as a human, inhibits a biological activity attributable to the level or presence of a compound or molecule of interest in the subject.

The term “antibody”, as used herein, refers to an immunoglobulin molecule which is able to specifically or selectively bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter can exist in a variety of forms. The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fᵥ, single chain Fᵥ, complementarity determining regions (CDRs), VL (light chain variable region), V_{H} (heavy chain variable region), Fab, F(ab′)₂ and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce F(ab′)₂ a dimer of Fab which itself is a light chain joined to V_{H}-C_{H1} by a disulfide bond. The F(ab′)₂ can be reduced under mild conditions to break the disulfide linkage in the hinge region, thereby converting the F(ab′)₂ dimer into an Fab₁ monomer. The Fab₁ monomer is essentially a Fab with part of the hinge region (see Paul, 1993). While various antibody fragments are defined in terms of the digestion of an intact antibody, one of skill will appreciate that such fragments can be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

An “antibody heavy chain”, as used herein, refers to the larger of the two types of polypeptide chains present in all intact antibody molecules.

An “antibody light chain”, as used herein, refers to the smaller of the two types of polypeptide chains present in all intact antibody molecules.

The term “single chain antibody” refers to an antibody wherein the genetic information encoding the functional fragments of the antibody are located in a single contiguous length of DNA. For a thorough description of single chain antibodies, see Bird et al., 1988; Huston et al., 1988).

The term “humanized” refers to an antibody wherein the constant regions have at least about 80% or greater homology to human immunoglobulin. Additionally, some of the nonhuman, such as murine, variable region amino acid residues can be modified to contain amino acid residues of human origin. Humanized antibodies have been referred to as “reshaped” antibodies. Manipulation of the complementarity-determining regions (CDR) is a way of achieving humanized antibodies. See for example, U.S. Pat. Nos. 4,816,567; 5,482,856; 6,479,284; 6,677,436; 7,060,808; 7,906,625; 8,398,980; 8,436,150; 8,796,439; and 10,253,111; and U.S. Patent Application Publication Nos. 2003/0017534, 2018/0298087, 2018/0312588, 2018/0346564, and 2019/0151448, each of which is incorporated by reference in its entirety.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response can involve either antibody production, or the activation of specific immunologically-competent cells, or both. An antigen can be derived from organisms, subunits of proteins/antigens, killed or inactivated whole cells or lysates.

The term “antimicrobial agents” as used herein refers to any naturally-occurring, synthetic, or semi-synthetic compound or composition or mixture thereof, which is safe for human or animal use as practiced in the methods of the presently disclosed subject matter, and is effective in killing or substantially inhibiting the growth of microbes. “Antimicrobial” as used herein, includes antibacterial, antifungal, and antiviral agents.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence can be complementary to regulatory sequences specified on the coding strand of a DNA molecule encoding a protein, which regulatory sequences control expression of the coding sequences. The antisense oligonucleotides of the presently disclosed subject matter include, but are not limited to, phosphorothioate oligonucleotides and other modifications of oligonucleotides.

The term “autologous”, as used herein, refers to something that occurs naturally and normally in a certain type of tissue or in a specific structure of the body. In transplantation, it refers to a graft in which the donor and recipient areas are in the same individual, or to blood that the donor has previously donated and then receives back, usually during surgery.

The term “basal medium”, as used herein, refers to a minimum essential type of medium, such as Dulbecco's Modified Eagle's Medium, Ham's F12, Eagle's Medium, RPMI, AR8, etc., to which other ingredients can be added. The term does not exclude media which have been prepared or are intended for specific uses, but which upon modification can be used for other cell types, etc.

The term “biocompatible”, as used herein, refers to a material that does not elicit a substantial detrimental response in the host.

The term “biodegradable”, as used herein, means capable of being biologically decomposed. A biodegradable material differs from a non-biodegradable material in that a biodegradable material can be biologically decomposed into units which can be either removed from the biological system and/or chemically incorporated into the biological system.

The term “biological sample”, as used herein, refers to samples obtained from a living organism, including skin, hair, tissue, blood, plasma, cells, sweat, and urine.

The term “bioresorbable”, as used herein, refers to the ability of a material to be resorbed in vivo. “Full” resorption means that no significant extracellular fragments remain. The resorption process involves elimination of the original implant materials through the action of body fluids, enzymes, or cells. Resorbed calcium carbonate can, for example, be redeposited as bone mineral, or by being otherwise re-utilized within the body, or excreted. “Strongly bioresorbable”, as the term is used herein, means that at least 80% of the total mass of material implanted is resorbed within one year.

The phrases “cell culture medium”, “culture medium” (plural “media” in each case), and “medium formulation” refer to a nutritive solution for cultivating cells and may be used interchangeably.

A “conditioned medium” is one prepared by culturing a first population of cells or tissue in a medium, and then harvesting the medium. The conditioned medium (along with anything secreted into the medium by the cells) can then be used in any desired way, such as to treat a disease or disorder in a subject, or to support the growth or differentiation of a second population of cells.

As used herein, the term “conservative amino acid substitution” is defined herein as an amino acid exchange within one of the five groups summarized in the following Table 2.

TABLE 2
Conservative Amino Acid Substitutions

| Group | Characteristics | Amino Acids |
|---|---|---|
| A. | Small aliphatic, nonpolar or slightly | Ala, Ser, Thr, Pro, Gly |
| | polar residues | |
| B. | Polar, negatively charged residues and | Asp, Asn, Glu, Gln |
| | their amides | |
| C. | Polar, positively charged residues | His, Arg, Lys |
| D. | Large, aliphatic, nonpolar residues | Met Leu, Ile, Val, Cys |
| E. | Large, aromatic residues | Phe, Tyr, Trp |

A “control” cell, tissue, sample, or subject is a cell, tissue, sample, or subject of the same type as a test cell, tissue, sample, or subject. The control can, for example, be examined at precisely or nearly the same time the test cell, tissue, sample, or subject is examined. The control can also, for example, be examined at a time distant from the time at which the test cell, tissue, sample, or subject is examined, and the results of the examination of the control can be recorded so that the recorded results can be compared with results obtained by examination of a test cell, tissue, sample, or subject. The control can also be obtained from another source or similar source other than the test group or a test subject, where the test sample is obtained from a subject suspected of having a disease or disorder for which the test is being performed.

A “test” cell, tissue, sample, or subject is one being examined or treated.

A “pathoindicative” cell, tissue, or sample is one which, when present, is an indication that the animal in which the cell, tissue, or sample is located (or from which the tissue was obtained) is afflicted with a disease or disorder. By way of example, the presence of one or more breast cells in a lung tissue of an animal is an indication that the animal is afflicted with metastatic breast cancer.

A tissue “normally comprises” a cell if one or more of the cells are present in the tissue in an animal not afflicted with a disease or disorder.

A “compound”, as used herein, refers to any type of substance or agent that is commonly considered a drug, or a candidate for use as a drug, combinations, and mixtures of the above, as well as polypeptides and antibodies of the presently disclosed subject matter.

“Cytokine”, as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets, and effector activities of these cytokines have been described.

“Chemokine”, as used herein, refers to an intercellular signaling molecule involved in the chemotaxis of white blood cells, such as T cells.

The term “delivery vehicle” refers to any kind of device or material, which can be used to deliver cells in vivo or can be added to a composition comprising cells administered to an animal. This includes, but is not limited to, implantable devices, aggregates of cells, matrix materials, gels, etc.

As used herein, a “derivative” of a compound refers to a chemical compound that can be produced from another compound of similar structure in one or more steps, as in replacement of H by an alkyl, acyl, or amino group.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” means an amount sufficient to produce a selected effect. A “therapeutically effective amount” means an effective amount of an agent being used in treating or preventing a disease or disorder.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, in some embodiments, at least about 100 to about 200 nucleotides, in some embodiments, at least about 200 nucleotides to about 300 nucleotides, yet in some embodiments, at least about 300 to about 350, in some embodiments, at least about 350 nucleotides to about 500 nucleotides, yet in some embodiments, at least about 500 to about 600, in some embodiments, at least about 600 nucleotides to about 620 nucleotides, yet in some embodiments, at least about 620 to about 650, and most in some embodiments, the nucleic acid fragment will be greater than about 650 nucleotides in length.

As used herein, a “functional” molecule is a molecule in a form in which it exhibits a property or activity by which it is characterized.

As used herein, a “functional biological molecule” is a biological molecule in a form in which it exhibits a property by which it is characterized. A functional enzyme, for example, is one which exhibits the characteristic catalytic activity by which the enzyme is characterized.

The term “growth factor” as used herein means a bioactive molecule that promotes the proliferation of a cell or tissue. Growth factors useful in the presently disclosed subject matter include, but are not limited to, transforming growth factor-alpha (TGF-α), transforming growth factor-beta (TGF-$), platelet-derived growth factors including the AA, AB and BB isoforms (PDGF), fibroblast growth factors (FGF), including FGF acidic isoforms 1 and 2, FGF basic form 2, and FGF 4, 8, 9, and 10, nerve growth factors (NGF) including NGF 2.5s, NGF 7.0s, and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), EG-VEGF, VEGF-related protein, Bv8, VEGF-E, granulocyte colony stimulating factor (G-CSF), insulin like growth factor (IGF) I and II, hepatocyte growth factor, glial neurotrophic growth factor, stem cell factor (SCF), keratinocyte growth factor (KGF), skeletal growth factor, bone matrix derived growth factors, and bone derived growth factors and mixtures thereof. Some growth factors may also promote differentiation of a cell or tissue. TGF, for example, may promote growth and/or differentiation of a cell or tissue.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul (1990) Methods for assessing the statistical significance of molecular sequence features by using general scoring schemes. Proc Natl Acad Sci USA 87:2264-2268, modified as in Karlin & Altschul (1993) Applications and statistics for multiple high-scoring segments in molecular sequences. Proc Natl Acad Sci USA 90:5873-5877). This algorithm is incorporated into the NBLAST and XBLAST programs (see Altschul et al. (1990a) Basic local alignment search tool. J Mol Biol 215:403-410; Altschul et al. (1990b) Protein database searches for multiple alignments. Proc Natl Acad Sci USA 87:14:5509-5513, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997) Gapped BLAST and PSI-BLAST: a new generation of protein database search programs. Nucleic Acids Res 25:3389-3402. Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “ingredient” refers to any compound, whether of chemical or biological origin, that can be used in cell culture media to maintain or promote the proliferation, survival, or differentiation of cells. The terms “component”, “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins, and the like. Other ingredients that promote or maintain cultivation of cells ex vivo can be selected by those of skill in the art, in accordance with the particular need.

The term “inhibit”, as used herein, refers to the ability of a compound, agent, or method to reduce or impede a described function, level, activity, rate, etc., based on the context in which the term “inhibit” is used. In some embodiments, inhibition is by at least 10%, in some embodiments by at least 25%, in some embodiments by at least 50%, and in some embodiments, the function is inhibited by at least 75%. The term “inhibit” is used interchangeably with “reduce” and “block”.

The term “inhibitor” as used herein, refers to any compound or agent, the application of which results in the inhibition of a process or function of interest, including, but not limited to, differentiation and activity. Inhibition can be inferred if there is a reduction in the activity or function of interest.

As used herein “injecting or applying” includes administration of a compound or composition of the presently disclosed subject matter by any number of routes and approaches including, but not limited to, topical, oral, buccal, intravenous, intratumoral, intramuscular, intra-arterial, intramedullary, intrathecal, intraventricular, transdermal, subcutaneous, intraperitoneal, intranasal, enteral, topical, sublingual, vaginal, ophthalmic, pulmonary, or rectal means.

As used herein, “injury” generally refers to damage, harm, or hurt; usually applied to damage inflicted on the body by an external force.

As used herein, an “instructional material” includes a publication, a recording, a diagram, or any other medium of expression, which can be used to communicate the usefulness of the composition of the presently disclosed subject matter in the kit for effecting alleviation of the various diseases or disorders recited herein. Optionally, or alternately, the instructional material may describe one or more methods of alleviating the diseases or disorders in a cell or a tissue of a mammal. The instructional material of the kit of the presently disclosed subject matter may, for example, be affixed to a container, which contains the identified compound presently disclosed subject matter, or be shipped together with a container, which contains the identified compound. Alternatively, the instructional material can be shipped separately from the container with the intention that the instructional material and the compound be used cooperatively by the recipient.

Used interchangeably herein are the terms “isolate” and “select”.

The terms “isolate”, “isolated”, “isolating”, and grammatical variations thereof when used in reference to cells, refers to a single cell of interest, or a population of cells of interest, at least partially isolated from other cell types or other cellular material with which it occurs in a culture or a tissue of origin. A sample is “substantially pure” when it is in some embodiments at least 60%, in some embodiments at least 75%, in some embodiments at least 90%, and, in certain cases, in some embodiments at least 99% free of cells or other cellular material other than cells of interest. Purity can be measured by any appropriate method, such as but not limited to those presented in the EXAMPLES.

An “isolated nucleic acid” refers to a nucleic acid segment or fragment, which has been separated from sequences, which flank it in a naturally occurring state, e.g., a DNA fragment that has been removed from the sequences, which are normally adjacent to the fragment, e.g., the sequences adjacent to the fragment in a genome in which it naturally occurs. The term also applies to nucleic acids, which have been substantially purified, from other components, which naturally accompany the nucleic acid, e.g., RNA or DNA, or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically binds to a target compound. A ligand (e.g., an antibody) “specifically binds to” or “is specifically immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow & Lane, 1988 for a description of immunoassay formats and conditions that can be used to determine specific immunoreactivity.

A “receptor” is a compound that specifically or selectively binds to a ligand.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule or bivalent group derived therefrom that joins two other molecules covalently or noncovalently, e.g., through ionic or hydrogen bonds or van der Waals interactions.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

Micro-RNAs are generally about 16-25 nucleotides in length. In some embodiments, miRNAs are RNA molecules of 22 nucleotides or less in length. These molecules have been found to be highly involved in the pathology of several types of cancer. Although the miRNA molecules are generally found to be stable when associated with blood serum and its components after EDTA treatment, introduction of locked nucleic acids (LNAs) to the miRNAs via PCR further increases stability of the miRNAs. LNAs are a class of nucleic acid analogues in which the ribose ring is “locked” by a methylene bridge connecting the 2′-O atom and the 4′-C atom of the ribose ring, which increases the molecule's affinity for other molecules. miRNAs are species of small non-coding single-stranded regulatory RNAs that interact with the 3′-untranslated region (3′-UTR) of target mRNA molecules through partial sequence homology. They participate in regulatory networks as controlling elements that direct comprehensive gene expression. Bioinformatics analysis has predicted that a single miRNA can regulate hundreds of target genes, contributing to the combinational and subtle regulation of numerous genetic pathways.

The term “modulate”, as used herein, refers to changing the level of an activity, function, or process. The term “modulate” encompasses both inhibiting and stimulating an activity, function, or process. The term “modulate” is used interchangeably with the term “regulate” herein.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double stranded DNA and cDNA. Furthermore, the terms, “nucleic acid”, “DNA”, “RNA” and similar terms also include nucleic acid analogs, i.e. analogs having other than a phosphodiester backbone. For example, the so called “peptide nucleic acids”, which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, intratumoral, and kidney dialytic infusion techniques.

“Permeation enhancement” and “permeation enhancers” as used herein relate to the process and added materials which bring about an increase in the permeability of skin to a poorly skin permeating pharmacologically active agent, i.e., so as to increase the rate at which the drug permeates through the skin and enters the bloodstream. “Permeation enhancer” is used interchangeably with “penetration enhancer”.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

A “prophylactic” treatment is a treatment administered to a subject who does not exhibit signs of a disease or injury or exhibits only early signs of the disease or injury for the purpose of decreasing the risk of developing pathology associated with the disease or injury.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide can serve a non-coding function (e.g., promoter, origin of replication, ribosome-binding site, etc.), as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

A “reversibly implantable” device is one which can be inserted (e.g., surgically or by insertion into a natural orifice of the animal) into the body of an animal and thereafter removed without great harm to the health of the animal.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

A “significant detectable level” is an amount of contaminate that would be visible in the presented data and would need to be addressed/explained during analysis of the forensic evidence.

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

By “small interfering RNAs (siRNAs)” is meant, inter alia, an isolated dsRNA molecule comprised of both a sense and an anti-sense strand. In some embodiments, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

As used herein, the term “secondary antibody” refers to an antibody that binds to the constant region of another antibody (the primary antibody).

As used herein, the term “single chain variable fragment” (scFv) refers to a single chain antibody fragment comprised of a heavy and light chain linked by a peptide linker. In some cases, scFv are expressed on the surface of an engineered cell, for the purpose of selecting particular scFv that bind to an antigen of interest.

The terms “solid support”, “surface” and “substrate” are used interchangeably and refer to a structural unit of any size, where said structural unit or substrate has a surface suitable for immobilization of molecular structure or modification of said structure and said substrate is made of a material such as, but not limited to, metal, metal films, glass, fused silica, synthetic polymers, and membranes.

By the term “specifically binds”, as used herein, is meant a molecule which recognizes and binds a specific molecule, but does not substantially recognize or bind other molecules in a sample, or it means binding between two or more molecules as in part of a cellular regulatory process, where said molecules do not substantially recognize or bind other molecules in a sample.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. “Standard” can also refer to an “internal standard”, such as an agent or compound which is added at known amounts to a sample and which is useful in determining such things as purification or recovery rates when a sample is processed or subjected to purification or extraction procedures before a marker of interest is measured. Internal standards are often but are not always limited to, a purified marker of interest which has been labeled, such as with a radioactive isotope, allowing it to be distinguished from an endogenous substance in a sample.

The term “stimulate” as used herein, means to induce or increase an activity or function level such that it is higher relative to a control value. The stimulation can be via direct or indirect mechanisms. In some embodiments, the activity or function is stimulated by at least 10% compared to a control value, in some embodiments by at least 25%, and in some embodiments by at least 50%. The term “stimulator” as used herein, refers to any composition, compound or agent, the application of which results in the stimulation of a process or function of interest.

A “subject” of diagnosis or treatment is an animal, including a human. It also includes pets and livestock.

As used herein, a “subject in need thereof” is a patient, animal, mammal, or human, who will benefit from a method or compositions of the presently disclosed subject matter.

As used herein, “substantially homologous amino acid sequences” includes those amino acid sequences which have at least about 95% homology, in some embodiments at least about 96% homology, more in some embodiments at least about 97% homology, in some embodiments at least about 98% homology, and most in some embodiments at least about 99% or more homology to an amino acid sequence of a reference sequence. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO4, 1 mM EDTA at 50° C. with washing in 2× standard saline citrate (SSC), 0.1% SDS at 50° C.; in some embodiments in 7% (SDS), 0.5 M NaPO₄, 1 mM EDTA at 50° C. with washing in 1×SSC, 0.1% SDS at 50° C.; in some embodiments 7% SDS, 0.5 M NaPO₄, 1 mM EDTA at 50° C. with washing in 0.5×SSC, 0.1% SDS at 50° C.; and more in some embodiments in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package, and the BLASTN or FASTA programs (Altschul et al., 1990a; Altschul et al., 1990b; Altschul et al., 1997). The default settings provided with these programs are suitable for determining substantial similarity of nucleic acid sequences for purposes of the presently disclosed subject matter.

The term “substantially pure” describes a compound, molecule, or the like that has been separated from components which naturally accompany it. Typically, a compound is substantially pure when at least 10%, more in some embodiments at least 20%, more in some embodiments at least 50%, more in some embodiments at least 60%, more in some embodiments at least 75%, more in some embodiments at least 90%, and most in some embodiments at least 99% of the total material (by volume, by wet or dry weight, or by mole percent or mole fraction) in a sample is the compound of interest. Purity can be measured by any appropriate method, e.g., those disclosed in the EXAMPLES, or in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

A “surface active agent” or “surfactant” is a substance that has the ability to reduce the surface tension of materials and enable penetration into and through materials.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

“Tissue” means (1) a group of similar cell united perform a specific function; (2) a part of an organism consisting of an aggregate of cells having a similar structure and function; or (3) a grouping of cells that are similarly characterized by their structure and function, such as muscle or nerve tissue.

The term “topical application”, as used herein, refers to administration to a surface, such as the skin. This term is used interchangeably with “cutaneous application” in the case of skin. A “topical application” is a “direct application”.

By “transdermal” delivery is meant delivery by passage of a drug through the skin or mucosal tissue and into the bloodstream. Transdermal also refers to the skin as a portal for the administration of drugs or compounds by topical application of the drug or compound thereto. “Transdermal” is used interchangeably with “percutaneous”.

The term “transfection” is used interchangeably with the terms “gene transfer”, “transformation”, and “transduction”, and means the intracellular introduction of a polynucleotide. “Transfection efficiency” refers to the relative amount of the transgene taken up by the cells subjected to transfection. In practice, transfection efficiency is estimated by the amount of the reporter gene product expressed following the transfection procedure.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “treating” may include prophylaxis of the specific injury, disease, disorder, or condition, or alleviation of the symptoms associated with a specific injury, disease, disorder, or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease. “Treating” is used interchangeably with “treatment” herein.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

The terminology used herein is for the purpose of describing the particular versions or embodiments only, and is not intended to limit the scope of the presently disclosed subject matter. All publications mentioned herein are incorporated by reference in their entirety.

### II. Representative Treatment Methods and Compositions

In some embodiments, the presently disclosed subject matter relates to methods for treating and/or inhibiting viral infections in a subject, which in some embodiments can be a human subject. In some embodiments, the methods comprises administering to a subject infected with and/or at risk for infection with a virus a composition comprising a glucosylceramidase inhibitor, a glucosylceramide synthase inhibitor, or any combination thereof, via a route and in an amount effective for treating and/or inhibiting the viral infection in the subject.

The methods of the presently disclosed subject matter can be employed to treat and/or inhibit viral infections related to several different viruses. By way of example and not limitation, the infection can be caused in some embodiments by an influenza A virus, in some embodiments by an influenza B virus, treating and/or inhibiting viral infections an influenza C virus, treating and/or inhibiting viral infections a vesicular stomatitis virus (VSV), treating and/or inhibiting viral infections an Ebola virus (EBOV), treating and/or inhibiting viral infections a measles virus, treating and/or inhibiting viral infections a coronavirus, which in some embodiments is COVID-19, or any combination thereof.

Additionally, the composition administered to the subject can include one or more of any glucosylceramidase inhibitors, glucosylceramide synthase inhibitors, and/or combinations thereof. Non-limiting examples of glucosylceramidase inhibitors include small molecule inhibitors, anti-glucosylceramidase antibodies and GBA-binding fragments and derivatives thereof, inhibitory nucleic acids that target glucosylceramidase gene products, and combinations thereof. Non-limiting examples of glucosylceramidase inhibitors include conduritol b epoxide (which is a mixture of 1-L-1,2-anhydro-myo-inositol and 1-D-1,2-anhydro-myo-inositol), D-threo-1-phenyl-2-palmitoylamino-3-pyrrolidino-1-propanol (also referred to as P4; see U.S. Pat. No. 8,168,587), castanospermine (International Union of Pure and Applied Chemistry (IUPAC) name (1S,6S,7R,8R,8aR)-octahydroindolizine-1,6,7,8-tetrol; see U.S. Pat. No. 5,066,807), isofagomine (IUPAC name 5R-(hydroxymethyl)-3R,4R-piperidinediol, mono 2S,3S-dihydroxybutanedioate; see U.S. Pat. Nos. 7,501,493 and 9,481,700), valienamine (IUPAC name (1S,2S,3R,6S)-6-Amino-4-(hydroxymethyl)cyclohex-4-ene-1,2,3-triol; see U.S. Pat. Nos. 4,486,602 and 7,365,192), validamine (IUPAC name (1R,2S,3S,4S,6R)-4-amino-6-(hydroxymethyl)cyclohexane-1,2,3-triol; see PCT International Patent Application Publication No. WO 2005/098014 and U.S. Patent Application Publication No. 2010/0151528), derivatives thereof, salts thereof, including but not limited to pharmaceutically acceptable salts thereof, and/or combinations thereof. Non-limiting examples of glucosylceramide synthase inhibitor include small molecules, anti-glucosylceramide synthase antibodies and UGCG-binding fragments and derivatives thereof, inhibitory nucleic acids that target a glucosylceramide synthase gene product, and any combinations thereof. In some embodiments, a glucosylceramide synthase inhibitor is selected from the group consisting of an iminosugar, miglustat (N-butyl-deoxynojirimycin; see U.S. Pat. No. 9,718,777), ibiglustat ([(3S)-1-azabicyclo[2.2.2]octan-3-yl] N-[2-[2-(4-fluorophenyl)-1,3-thiazol-4-yl]propan-2-yl]carbamate; see U.S. Patent Application Publication No. 2019/0248790), eliglustat (N-[(1R,2R)-1-(2,3-Dihydro-1,4-benzodioxin-6-yl)-1-hydroxy-3-(1-pyrrolidinyl)-2-propanyl]octanamide; see U.S. Pat. Nos. 6,916,802; 7,196,205; 7,253,185; and 7,615,573), 1-phenyl-2-palmitoylamino-3-morpholino-1-propanol, derivatives thereof, salts thereof, including but not limited to pharmaceutically acceptable salts thereof, and/or combinations thereof. In some embodiments, subject to whom the presently disclosed methods are applied is a human subject.

In some embodiments, non-limiting examples of glucosylceramidase inhibitors include anti-glucosylceramidase antibodies and GBA-binding fragments and derivatives thereof. In some embodiments, the GBA is a human GBA, which in some embodiments can have an amino acid sequence as set forth in any of SEQ ID NOs: 2, 4, 6, 8, and 10. SEQ ID NOs: 2, 4, 6, 8, and 10 differ at their N-termini, with each one being identical in its C-terminal 384 amino acids (compare e.g., amino acids 153-536 of SEQ ID NOs: 2, 4, and 6, amino acids 66-449 of SEQ ID NO: 8, and amino acids 123-486 of SEQ ID NO: 10). As such, an anti-glucosylceramidase antibody or a fragment or derivative thereof that binds to an epitope within the C-terminal 384 amino acids of a GBA polypeptide of and of SEQ ID NOs: 2, 4, 6, 8, and 10 would be expected to modulate a biological activity of any of these polypeptides. If an anti-glucosylceramidase antibody or a fragment or derivative thereof was desired that modulated a biological activity of a subset of the GBA polypeptides of SEQ ID NOs: 2, 4, 6, 8, and 10, such an anti-glucosylceramidase antibody or a fragment or derivative thereof can be designed to bind to an epitope found in the N-terminal 152 amino acids of any of SEQ ID NOs: 2, 4, and 6, the N-terminal 65 amino acids of SEQ ID NO: 8, and/or the N-terminal 122 amino acids of SEQ ID NO: 10. In some embodiments, an anti-glucosylceramidase antibody or a fragment or derivative thereof bind to and as a consequence inhibits the biological activity of the GBA polypeptide, for example a biological activity involved in catalyzing the conversion of glucosylceramide (GlcCer) to ceramide.

Additional glucosylceramide synthase inhibitors for use in the compositions and methods of the presently disclosed subject matter include, for example, those described in PCT International Patent Application Publication Nos. WO 2015/089067, WO 2014/151291, WO 2014/043068, WO 2008/150486, WO 2010/014554, WO 2012/129084, WO 2011/133915, and WO 2010/091164; U.S. Pat. Nos. 9,126,993; 8,961,959; 8,940,776; 8,729,075; 8,309,593; and U.S. Patent Application Publication Nos. 2014/0255381 and 2014/0336174; each of which is incorporated herein by reference in its entirety. Additional ceramidase inhibitors for use in the compositions and methods of the presently disclosed subject matter include, for example, those described in PCT International Patent Application Publication Nos. WO 2015/173168 and WO 2015/173169, each of which is incorporated herein by reference in its entirety.

In some embodiments, non-limiting examples of glucosylceramidase (GBA) inhibitors include anti-glucosylceramidase antibodies and GBA-binding fragments and derivatives thereof. In some embodiments, the GBA is a human GBA, which in some embodiments can have an amino acid sequence as set forth in any of SEQ ID NOs: 2, 4, 6, 8, and 10. SEQ ID NOs: 2, 4, 6, 8, and 10 differ at their N-termini, with each one being identical in its C-terminal 384 amino acids (compare e.g., amino acids 153-536 of SEQ ID NOs: 2, 4, and 6, amino acids 66-449 of SEQ ID NO: 8, and amino acids 123-486 of SEQ ID NO: 10). As such, an anti-glucosylceramidase antibody or a fragment or derivative thereof that binds to an epitope within the C-terminal 384 amino acids of a GBA polypeptide of and of SEQ ID NOs: 2, 4, 6, 8, and 10 would be expected to modulate a biological activity of any of these polypeptides. If an anti-glucosylceramidase antibody or a fragment or derivative thereof was desired that modulated a biological activity of a subset of the GBA polypeptides of SEQ ID NOs: 2, 4, 6, 8, and 10, such an anti-glucosylceramidase antibody or a fragment or derivative thereof can be designed to bind to an epitope found in the N-terminal 152 amino acids of any of SEQ ID NOs: 2, 4, and 6, the N-terminal 65 amino acids of SEQ ID NO: 8, and/or the N-terminal 122 amino acids of SEQ ID NO: 10. In some embodiments, an anti-glucosylceramidase antibody or a fragment or derivative thereof bind to and as a consequence inhibits the biological activity of the GBA polypeptide, for example a biological activity involved in catalyzing the conversion of glucosylceramide (GlcCer) to ceramide (see FIG. 1).

In some embodiments, non-limiting examples of glucosylceramide synthase (UGCG) inhibitors include anti-glucosylceramide synthase antibodies and UGCG-binding fragments and derivatives thereof. In some embodiments, the UGCG is a human UGCG, which in some embodiments can have an amino acid sequence as set forth in SEQ ID NO: 12. As such, an anti-glucosylceramide synthase antibody or a fragment or derivative thereof that binds to an epitope within a UGCG polypeptide of SEQ ID NO: 2, 4, 6, 8, and 12 would be expected to modulate a biological activity of the UGCGV polypeptide. In some embodiments, an anti-glucosylceramide synthase antibody or a fragment or derivative thereof binds to and as a consequence inhibits the biological activity of the UGCG polypeptide, for example a biological activity involved in catalyzing the conversion of ceramide to glucosylceramide (see FIG. 1).

In some embodiments, non-limiting examples of glucosylceramidase (GBA) inhibitors and/or anti-glucosylceramide synthase inhibitors include anti-glucosylceramidase and/or anti-glucosylceramide synthase inhibitory nucleic acids. As used herein, the phrase “inhibitory nucleic acid” refers to any nucleic acid molecule capable of mediating RNA interference (RNAi) or gene silencing. See e.g., Bass (2001) Nature 411:428-429; Elbashir et al. (2001) Nature 411:494-498; and PCT International Publication Nos. WO 99/07409; WO 99/32619; WO 00/01846; WO 00/44895; WO 00/44914; WO 01/36646; WO 01/29058. Exemplary inhibitory nucleic acids include small interfering RNAs, short interfering RNAs, siRNAs, and miRNAs. In some embodiments, the inhibitory nucleic acid comprises a double stranded polynucleotide molecule comprising complementary sense and antisense regions, wherein the antisense region comprises a sequence complementary to a region of a target nucleic acid molecule (for example, an mRNA encoding GBA or UGCG). In some embodiments, the inhibitory nucleic acid comprises a single stranded polynucleotide having self-complementary sense and antisense regions, wherein the antisense region comprises a sequence complementary to a region of a target nucleic acid molecule. In some embodiments, the inhibitory nucleic acid comprises a single stranded polynucleotide having one or more loop structures and a stem comprising self complementary sense and antisense regions, wherein the antisense region comprises a sequence complementary to a region of a target nucleic acid molecule, and wherein the polynucleotide can be processed either in vivo or in vitro to generate an active inhibitory nucleic acid capable of mediating RNAi. As used herein, inhibitory nucleic acid molecules need not be limited to those molecules containing only RNA, but further encompass chemically modified nucleotides and non-nucleotides.

In some embodiments, non-limiting examples of glucosylceramidase (GBA) inhibitors and/or anti-glucosylceramide synthase (UGCG) inhibitors include anti-glucosylceramidase and/or anti-glucosylceramide synthase inhibitory nucleic acids that target a GBA or a UGCG nucleic acid. Exemplary nucleic acids that can be targeted include the human GBA nucleic acids as set forth in SEQ ID NOs: 1, 3, 5, 7, and 9, and the human UGCG nucleic acid as set forth in SEQ ID NO: 11. Methods to design inhibitor nucleic acids are known in the art.

In some embodiments, CRISPR/Cas9 Gene Editing is employed to inhibit GBA and/or UGCG. Exemplary techniques for targeting GBA and UGCG are disclosed herein below. gRNAs that can be targeting GBA and UGCG can be selected using the CRISPR design tool developed by the Feng Zhang laboratory at the Massachusetts Institute of Technology (Cambridge, Mass., United States of America), and include but are not limited to SEQ ID NO: 15 (GBA) or SEQ ID NO: 16 (UGCG).

In some embodiments, the presently disclosed subject matter also relates to methods for inhibiting vial infections of cells, which in some embodiments can involve contacting a cell with a composition comprising a glucosylceramidase inhibitor, a glucosylceramide synthase inhibitor, or any combination thereof in an amount sufficient for inhibiting infection of the cell with the virus. In some embodiments, the virus is selected from the group consisting of an influenza A virus, an influenza B virus, an influenza C virus, a vesicular stomatitis virus (VSV), an Ebola virus (EBOV), a measles virus, a coronavirus, optionally COVID-19, and any combination thereof. In some embodiments the glucosylceramidase inhibitors and the glucosylceramidase inhibitors are as set forth herein above. In some embodiments, the cell is a human cell, optionally a human cell present within a subject.

In some embodiments, the presently disclosed subject matter also relates to methods for inhibiting endosomal fusion of a virus in a cell, which in some embodiments comprises contacting a cell with a glucosylceramidase inhibitor, a glucosylceramide synthase inhibitor, or any combination thereof as disclosed herein. In some embodiments, the amount of the glucosylceramidase inhibitor, the glucosylceramide synthase inhibitor, and/or the combination thereof employed is effective for inhibiting endosomal fusion of the virus in the cell.

In some embodiments, the presently disclosed subject matter also relates to compositions, optionally pharmaceutical compositions, for use in the methods disclosed herein. In some embodiments, composition comprises at least one glucosylceramidase inhibitor as disclosed herein and/or at least one glucosylceramide synthase inhibitor as disclosed herein, or any combination thereof. In some embodiments, the composition is effective for treating and/or inhibiting the viral infection in a subject and/or for inhibiting infection of the cell with the virus.

In some embodiments, the at least one glucosylceramidase inhibitor as disclosed herein and/or at least one glucosylceramide synthase inhibitor as disclosed herein, and/or any combination thereof of the presently disclosed subject matter can be provided in a composition that includes a carrier, particularly a pharmaceutically acceptable carrier, such as but not limited to a carrier pharmaceutically acceptable in humans. Any suitable pharmaceutical formulation can be used to prepare the compositions for administration to a subject.

For example, suitable formulations can include aqueous and non-aqueous sterile injection solutions that can contain anti-oxidants, buffers, bacteriostatics, bactericidal antibiotics, and solutes that render the formulation isotonic with the bodily fluids of the intended recipient.

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

The methods and compositions of the presently disclosed subject matter can be used with additional adjuvants or biological response modifiers including, but not limited to, cytokines and other immunomodulating compounds.

In some embodiments, therapeutic agents, including, but not limited to, cytotoxic agents, anti-angiogenic agents, pro-apoptotic agents, antibiotics, hormones, hormone antagonists, chemokines, drugs, prodrugs, toxins, enzymes or other agents may be used as adjunct therapies when using the compositions described herein. Drugs useful in the presently disclosed subject matter may, for example, possess a pharmaceutical property selected from the group consisting of antimitotic, antikinase, alkylating, antimetabolite, antibiotic, alkaloid, anti-angiogenic, pro-apoptotic agents, and combinations thereof.

In some embodiments, the presently disclosed compositions and methods can further comprise administering to the subject at least one additional immunosuppressive agent to a subject. In some embodiments, the at least one additional immunosuppressive agent is selected from the group consisting of methotrexate, cyclophosphamide, cyclosporine, cyclosporin A, chloroquine, hydroxychloroquine, sulfasalazine (sulphasalazopyrine), a gold salt, D-penicillamine, leflunomide, azathioprine, anakinra, infliximab (REMICADE), etanercept, a TNFα blocker, a non-steroidal anti-inflammatory drug (NSAID), or any combination thereof. In some embodiments, the NSAID is selected from the group consisting of acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, a cyclooxygenase-2 (Cox-2) inhibitor, tramadol, rapamycin (sirolimus), an analog thereof, or any combination thereof.

The compositions of the presently disclosed subject matter can be administered by any route of administration reasonably expected to deliver the compositions to a desired target site, including but not limited to a site of infection or possible infection with a virus. Suitable methods for administration of the compositions of the presently disclosed subject matter thus include, but are not limited to intravenous administration and delivery directly to the target tissue or organ. In some embodiments, the method of administration encompasses features for regionalized delivery or accumulation of the composition at the site in need of treatment. In some embodiments, the composition is/are delivered directly into the lung. In some embodiments, selective delivery of the composition is accomplished by intravenous injection of composition, where they accumulate in the lung. Other modes of administration that can be employed include topical, oral, buccal, intramuscular, intra arterial, intramedullary, intrathecal, intraventricular, transdermal, subcutaneous, intraperitoneal, intranasal, enteral, topical, sublingual, vaginal, ophthalmic, pulmonary, or rectal means. Compounds or agents of the presently disclosed subject matter can be administered to a subject by one or more of these routes when appropriate. In some embodiments, intratracheal installation, insufflation, nebulization, dry powder inhalation, aerosol inhalation, and combinations thereof are employed as a route or routes of administration of the composition of the presently disclosed subject matter.

Where the administration of the composition is by injection or direct application, the injection or direct application may be in a single dose or in multiple doses. Where the administration of the composition is by infusion, the infusion may be a single sustained dose over a prolonged period of time or multiple infusions.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

A pharmaceutical composition of the presently disclosed subject matter may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the presently disclosed subject matter may further comprise one or more additional pharmaceutically active agents. Particularly contemplated additional agents include anti-emetics and scavengers such as cyanide and cyanate scavengers.

Controlled- or sustained-release formulations of a pharmaceutical composition of the presently disclosed subject matter may be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Genaro, ed., 1985, Remington's Pharmaceutical Sciences, Mack Publishing Co., Easton, Pa., which is incorporated herein by reference.

An effective dose of a composition of the presently disclosed subject matter is administered to a subject in need thereof. A “treatment effective amount”, “therapeutic amount”, or “therapeutically effect amount” is an amount of a therapeutic composition sufficient to produce a measurable response (e.g., a biologically or clinically relevant response in a subject being treated). In some embodiments, an activity that inhibits a viral infection is measured. Actual dosage levels of active ingredients in the compositions of the presently disclosed subject matter can be varied so as to administer an amount of the active compound(s) that is effective to achieve the desired therapeutic response for a particular subject. The selected dosage level will depend upon the activity of the therapeutic composition, the route of administration, combination with other drugs or treatments, the severity of the condition being treated, and the condition and prior medical history of the subject being treated. However, it is within the skill of the art to start doses of the compound at levels lower than required to achieve the desired therapeutic effect and to gradually increase the dosage until the desired effect is achieved. The potency of a composition can vary, and therefore a “treatment effective amount” can vary. However, using generally applicable assay methods, one skilled in the art can readily assess the potency and efficacy of a candidate compound of the presently disclosed subject matter and adjust the therapeutic regimen accordingly. After review of the disclosure of the presently disclosed subject matter presented herein, one of ordinary skill in the art can tailor the dosages to an individual subject, taking into account the particular formulation, method of administration to be used with the composition, and particular disease treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

As such, in some embodiments the presently disclosed composition thereof is/are present in a pharmaceutically acceptable carrier, which in some embodiments can be a pharmaceutically acceptable for use in humans.

Typically, dosages of the compound of the presently disclosed subject matter which may be administered to an animal, in some embodiments a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. In one aspect, the dosage of the compound will vary from about 1 mg to about 10 g per kilogram of body weight of the animal. In another aspect, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

Suitable preparations include injectables, either as liquid solutions or suspensions, however, solid forms suitable for solution in, suspension in, liquid prior to injection, may also be prepared. The preparation may also be emulsified, or the active agent(s) encapsulated in nanoparticles and/or microparticles (including but not limited to liposomes). The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants.

Compositions and methods for encapsulating active agents in nanoparticles and/or microparticles (including but not limited to liposomes) are disclosed, for example, in U.S. Pat. No. 9,867,888 and U.S. Patent Application Publication Nos. 2018/0140717, 2018/0147298, 2018/0148719, 2018/0177727, 2018/0221402, and 2019/0345492, each of which is incorporated herein by reference in its entirety.

In some embodiments, the presently disclosed subject matter provides use of pharmaceutical compositions comprising, consisting essentially of, or consisting of an effective amount of a composition comprising a glucosylceramidase inhibitor, a glucosylceramide synthase inhibitor, or any combination thereof for treating and/or inhibiting a viral infection in a subject and/or for inhibiting viral infections of cells, and/or for inhibiting endosomal fusion of viruses in cells.

In some embodiments, the presently disclosed subject matter provides use of an effective amount of a composition comprising, consisting essentially of, or consisting of an effective amount of a composition comprising a glucosylceramidase inhibitor, a glucosylceramide synthase inhibitor, or any combination thereof for the preparation of a medicament to treat and/or inhibit a viral infection in a subject, and/or to inhibit viral infections of cells, and/or to inhibit endosomal fusion of viruses in cells.

### Examples

The presently disclosed subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### Materials and Methods for Examples

Cells. HEK 293 (human embryonic kidney; ATCC CRL-1573; American Type Culture Collection, Manassas, Va., United States of America), HEK 293T/17 (ATCC CRL-11268), A549 (human lung carcinoma; ATCC CCL-185), and BHK-21 (baby hamster kidney; ATCC CCL-10) cells were grown in Dulbecco modified Eagle medium (DMEM) supplemented with 10% fetal bovine serum (FBS), 1% sodium pyruvate, 1% antibiotic/antimycotic, and 1% L-glutamine at 37° C. (all from Gibco Life Technologies, Carlsbad, Calif., United States of America) with 5% CO₂.

CRISPR/Cas9 Gene Editing. gRNAs targeting GBA and UGCG were selected using the CRISPR design tool developed by the Feng Zhang laboratory at the Massachusetts Institute of Technology (Cambridge, Mass., United States of America) and available at via their website. The gRNAs were cloned into a Cas9-sgRNA (Plasmid No. 68463, deposited by Su-Chun Zhang with Addgene, Watertown, Mass., United States of America) using restriction endonuclease BbsI. The resulting plasmids along with a plasmid encoding GFP were individually co-transfected into HEK 293 and A549 cells and sorted for positive GFP expression into single cell colonies using an Influx flow cytometer. The cells, originally in wells of 96-well plates, were expanded, and the DNA from over 100 discrete colonies was extracted and analyzed by PCR and subsequent gel electrophoresis. PCR was performed using primers flanking the regions of interest and designed to produce fragments of ˜250-300 bp. Colonies with PCR products indicative of CRISPR activity (˜5-10 per cell line) as compared to products from WT cells were maintained and analyzed by western blotting (for GBA protein) and mass spectrometry (for sphingolipid content). For UGCG, colonies were analyzed for shifts in UGCG DNA fragment size by PCR, and then 5-10 colonies per cell line were analyzed by mass spectrometry (for enzyme activity and sphingolipid content) and western blotting (for UGCG protein). No difference in growth rate was seen for WT and UGCG 293/A549 KO cells over the time period analyzed (4 days). Growth rates were measured by MTS assay over a period of four days.

Inhibitors and Other Reagents. Epidermal growth factor (EGF; Catalog No. E9644), DL-threo-1-phenyl-2-palmitoylamino-3-morpholino-1-propanol (PPMP; Catalog No. P4194), and Bafilomycin A1 (Catalog NO. B1793) were purchased from Sigma-Aldrich Corp. (St. Louis, Mo., United States of America). EGF-Alexa Fluor 555 (Catalog No. E35350) was purchased from Thermo Fischer Scientific. Inc. (Waltham, Mass., United States of America). BbsI was purchased from New England Biolabs (Ipswich, Mass., United States of America; Catalog No. 391 R0539S).

Influenza Viruses and VLPs, VSV Pseudoviruses, and EBOV trVLPs. Stocks of PR8 IAV were obtained from Charles River Laboratories (Wilmington, Mass., United States of America). PR8 NS-GFP was kindly provided by Dr. Thomas Braciale at the University of Virginia (Charlottesville, Va., United States of America: see also Hufford et al., 2012). All influenza viruses were grown in embryonated chicken eggs, thereby cleaving HA0 before any infection assays were performed (see Gotoh et al., 1990; Gotoh et al., 1992).

VSV-GFP pseudoviruses were produced using 5×10⁵ BHK-21 cells plated in forty 10 cm² dishes. The cells were transfected (when ˜75-80% confluent) with plasmids encoding, as indicated, VSV-G (plasmid backbone: pCAGGS), EBOV-GPΔ (plasmid backbone: VRC6002), or Measles F and H (plasmid backbone: PCXN2), with polyethylenimine (PEI; Catalog No. 23966, Polysciences, Inc., Warrington, Pa., United States of America). Measles F and H (Edmonston strain) plasmids were generously provided by Dr. Yusuke Yanagi of Kyushu University (Fukuoka, Japan; see Tatsuo et al., 2000). This strain of measles virus was reported to use CD46 as its receptor on non-lymphoid cells (Tatsuo et al., 2000). 24 hours later, cells were infected with pre-titered VSV-ΔG helper virus encoding GFP (from a plaque eluate) at 37° C. for 1 hour, washed extensively with PBS, and then cultured in growth medium overnight at 37° C. 24 hours post-infection with helper virus, cell supernatants containing budded pseudovirus were collected, centrifuged twice (1360×g/10 minutes) to clear debris, and concentrated ˜50-fold using a Viva-Spin 20 300 kDa concentrator (Sigma-Aldrich). Finally, the concentrated pseudovirus was centrifuged through a 20% sucrose cushion in HEPES-MES (HM) buffer containing 20 mM HEPES, 20 mM MES, 130 mM NaCl, pH 7.4) in an SW28 rotor for 2 hours at 112,398×g at 4° C. and then resuspended in 10% sucrose-HM. Pseudovirus stocks were stored at ˜80° C.

VSV-ΔG helper virus was produced as described previously (see Whitt, 2010). In brief, 5×10⁵ BHK-21 cells plated in five 10 cm² dishes were transfected at ˜75-80% confluency with 12 μg (per dish) of plasmid expressing VSV-G using PEI. ˜24 hours later, the cells were infected with ˜40 μl of VSV-GFP plaque eluate (3.39×10⁸ infectious units/mL) in serum-free media for 1 hour at 37° C. Cells were then washed extensively with PBS and incubated overnight in complete media at 37° C. The next day, supernatants containing helper virus were collected, centrifuged for 10 minutes at 1070×g to clear debris, and stored at −80° C.

Influenza M1-VLPs were produced by transfecting 1×10⁶ HEK 293T/17 cells in each of five 10 cm² dishes in complete media with no antibiotic/antimycotic using plasmids encoding βlaM1, and either WSN HA+WSN NA (plasmid backbone: pCAGGS), VSV-G, or EBOV-GPΔ using PEI. WSN is an H1N1 strain of influenza that is trypsin-independent in vitro (see Sun et al., 2010). The βlaM1 plasmid was kindly provided by Dr. Adolfo Garcia-Sastre and the NIAID Centers of Excellence for Influenza Research and Surveillance (CEIRS) program (Tscherne et al., 2010). Media containing VLPs was harvested 24 and 48 hours post transfection, pooled, and centrifuged twice to clear debris. The VLPs were then pelleted through a 20% sucrose cushion in HM buffer using an SW28 rotor for 2 hours at 112,398×g at 4° C., and then resuspended in 10% sucrose-HM. VLPs were stored at −80° C.

Transcription/replication-competent viral-like particles (trVLPs) were prepared as described in Nelson et al., 2016. Briefly, HEK 293T/17 cells were transfected with pCAGGS-L, a tetracistronic minigenome plasmid, pCAGGS-VP35, pCAGGS-NP, pCAGGS-VP30, and pCAGGS-T7 polymerase. 24 hours post transfection the medium was replaced with fresh growth medium containing 5% FBS and cells were incubated at 37° C. 72 hours post transfection the medium was harvested, pooled, and centrifuged for 5 minutes at 800×g to clear cellular debris.

IAV Reporter Infection Assay. Cells were seeded in 96 well plates at a density of 3×10⁴ cells per well. The next day cells were prechilled to 4° C. for 15 minutes and then incubated with PR8 influenza encoding GFP fused to the N-terminus of NS1 (MOI of ˜1) in growth medium without FBS or trypsin and centrifuged at 250×g for 1 hour at 4° C. The cells were then incubated at 37° C. Approximately 16-18 hours post infection, cells were lifted with trypsin, fixed in 4% paraformaldehyde (PFAM), and assayed for GFP signal on an Attune NxT flow cytometer (Thermo Fisher Scientific). Uninfected cells were used to set a background value for GFP. For infection assays in the presence of inhibitors, cells were pre-treated with 20 μM PPMP for 48 hours or 100 nM Bafilomycin for 1 hour before adding PR8 in the presence of the indicated inhibitor. All values were normalized to mock infected cells.

qPCR. Cells were seeded in 24 well plates at a density of 5×10⁴ cells per well. The next day cells were prechilled to 4° C. for 15 minutes and then incubated with WT PR8 influenza in growth medium without FBS (with or without 1 μg/mL trypsin as indicated) and centrifuged at 250×g for 1 hour at 4° C. The cells were then incubated at 37° C. At the indicated time points samples were harvested and RNA extracted using TRIzol reagent according to manufacturer's instructions (Catalog No. 15596026; Thermo Fisher Scientific). cDNA was generated using iScript cDNA synthesis (Catalog No. 1708891; Bio-Rad) according to manufacturer's instructions, and qPCR was performed with the following primers: IAV M1 forward (5′-CTTCTAACCGAGGTCGAAACG-3′; SEQ ID NO: 13) and reverse (5′-GGCATTTTGGACAAAGCGTCTA-3′; SEQ ID NO: 14). Relative expression of IAV M1 mRNA was calculated after normalization to endogenous reference gene beta-2-microglobin (Catalog No. pHSACID0015347; Bio-Rad).

Influenza M1-VLP Entry Assay. Cells were seeded in 96 well plates at a density of 3×10⁴ cells per well. The next day cells were prechilled to 4° C. for 15 minutes and then incubated with previously titered influenza M1-VLPs diluted in Opti-MEM I (OMEM) and centrifuged at 250×g for 1 hour at 4° C. The cells were incubated at 37° C. for 3 hours before addition of the βlaM substrate CCF2-AM (Catalog No. K1032; Invitrogen Corp., Carlsbad, Calif., United States of America) in loading buffer (phenol red-free DMEM, 5 mM (HEK 293) or 20 mM (A549) probenecid (Catalog NO. 156370; MP Biomedicals, Solon, Ohio, United States of America), 2 mM L glutamine, 25 mM HEPES, 200 nM bafilomycin) and incubated for an additional hour at room temperature. Cells were then washed with PBS and allowed to incubate in loading buffer with 10% FBS overnight in the dark at room temperature. The following day, cells were lifted with trypsin, fixed in 4% PFAM, and analyzed for VLP entry as measured by CCF2-AM cleavage (resulting in FRET disruption and a color shift from green (518 nm) to blue (447 nm)) on an Attune NxT flow cytometer (Thermo Fisher Scientific).

VSV Pseudovirus Infection Assay. Cells were seeded in 96 well plates at a density of 3×10⁴ cells per well. The next day cells were prechilled to 4° C. for 15 minutes and then incubated with VSV pseudoviruses in Opti-MEM I (OMEM) and centrifuged at 250×g for 1 hour at 4° C. Cells were then washed and incubated for 18-24 hours at 37° C. The cells were then lifted, fixed, and analyzed for GFP expression via flow cytometry on an Attune NxT flow cytometer (Thermo Fisher Scientific).

trVLP Infection Assay. Infection of HEK 293 cells by trVLPs was performed as described previously (Nelson et al., 2016). Cells were seeded in opaque 96 well plates, and when the cells were approximately 50% confluent they were transfected with (per well) 13.88 ng pCAGGS-Tim1, 4.16 ng pCAGGS-VP30, 6.94 ng pCAGGS-VP35, 6.94 ng pCAGGS-NP, and 55.55 ng pCAGGS-L in order to support entry and replication of infecting trVLPs. 24 hours post transfection the medium was removed and trVLPs were added. Cells were incubated for 18-24 hours in growth medium at 37° C., before being analyzed using Renilla-Glo luciferase assay system (Catalog No. E2710; Promega Corporation, Madison, Wis., United States of America) on a GLOMAX® brand plate reader (Promega Corporation).

VSV Pseudovirus Infection Assay. 3×10⁴ cells were seeded per well in 96 well plates. 24 hours later the cells were incubated with VSV pseudoviruses in Opti-MEM I (OMEM) followed by centrifugation at 4° C. for 1 hour at 250×g. The cells were washed and then incubated in growth medium at 37° C. for 18-24 hours after which they were lifted, fixed, and analyzed for GFP expression on an Attune NxT flow cytometer.

Western Blotting. Cell samples were lysed with PBS containing 1% SDS, 5 mM EDTA, and 1 mM sodium vanadate (Catalog No. S6508; Sigma-Aldrich). These lysates were then resolved by SDS-PAGE and the proteins subsequently transferred to PVDF membranes. The membranes were then probed with the indicated primary antibodies followed by secondary antibodies coupled to horseradish peroxidase. Signals were visualized following incubation with a chemiluminescent HRP substrate. Images were captured with an Alpha Innotech FluorChem detector (Alpha Innotech Corp., San Leansro, Calif., United States of America). For quantification, samples were normalized to signals for GAPDH in the lysates using IMAGE STUDIO™ Lite brand image processing software (LI-COR Biosciences, Lincoln, Nebr., United States of America).

Antibodies. Antibodies were purchased from the following sources: anti-EGFR (A-10; Santa Cruz Biotechnology, Santa Cruz, Calif., United States of America; Catalog No, sc-373746); anti-GBA (Abcam, Cambridge, Mass., United States of America; Catalog No. ab55080); anti-GAPDH (14C10) (Cell Signaling Technology, Inc., Danvers, Mass., United States of America; Catalog No. 3683); anti-Cathepsin B (Santa Cruz Biotechnology; Catalog No. sc-365558); anti-UGCG (M03) (Abnova Corp., Taipei City, Taiwan; Catalog No. H00007357).

Preparation of C6 Ceramide Nanoliposomes. C6 ceramide nanoliposomes were prepared as described previously (Ryland et al., 2013). Briefly, 1,2-distearoyl-sn-glycero-3-phosphocholine, 1,2-distearoyl-sn-glycero-3-phosphoethanolamine-N-[methoxy polyethyleneglycol-2000], 1,2-dioleoyl-sn-glycero-3-phosphoethanolamine, N-octanoyl-sphingosine-1-[succinyl(methoxypolyethylene glycol-750)] (PEG(750)-C₈), and N-hexanoyl-d-erythro-sphingosine (C₆-ceramide) were combined in chloroform at a molar ratio of 3.75:3:1.75:0.75:0.75. The lipid mixture was dried and then rehydrated followed by sonication and extraction through a 100 nm polycarbonate membrane.

Lipid Mass Spectrometry and Enzyme Activity Assay. Lipids were extracted from cell lysates and analyzed on an Acquity I-Class/Xevo TQ-S micro IVD system (Waters Corp., Milford, Mass., United States of America) as described previously (Hankins et al., 2011). Mass spectrometry peaks were compared to internal standards and all data are represented as pmol of lipid/mg of protein. For the UGCG enzyme activity assay, cells were incubated with 5 μM C₆ ceramide nanoliposomes (˜100 nm) for 4 hours, collected, and then subjected to lipid extraction as described previously (Drake et al., 2017). Lipid species were then analyzed, as described previously, on an Acquity I-Class/Xevo TQ-S system [54]. Internal standards were compared to mass spectrometry peaks and all data are represented as pmol of lipid/mg of protein.

EGFR Degradation. Cells were seeded in 6 well plates at a density of 6×10⁵ cells per well. The next day cells were washed twice with PBS and then incubated with 50 ng/mL EGF in growth media at 37° C. for the indicated times without the presence of cycloheximide. Cells were lysed and analyzed by western blotting as described above. For quantification, samples were normalized to the signal for GAPDH and then to 0 hour.

Cathepsin Activity Assays. In vitro Cathepsin B and Cathepsin L activities in cell lysates were measured as described previously (Ebert et al., 2002; Johansen et al., 2013). Briefly, Cathepsin L activity was assayed with the Cathepsin B+L substrate Z-Phe-Arg-7-AMC (CAS No. 70382-26-2; Catalog No. 03-32-1501; Calbiochem, available from Sigma-Aldrich) in the presence of 1 μM CA-074 (Calbiochem Catalog No. 205530), a Cathepsin B inhibitor. Cathepsin B was measured in the same manner using Z-Arg-Arg-7-AMC (Calbiochem Catalog No. 219392) and no inhibitor. In vivo Cathepsin B activity was measured using Magic Red-(RR)₂ (Catalog No. ICT937; BioRad) stain as per the manufacturer's instructions.

Influenza Fusion Assay. Influenza PR8 was dually labeled as described previously (Jan et al., 2000; Pewzner-Jung et al., 2010) with 3,3′-dioctadecyloxacarbocyanine (DiOC18) and octadecyl rhodamine B (R18) at final concentrations of 0.2 and 0.4 μM, respectively. The reaction mixture was vortexed vigorously and left to incubate for one hour at room temperate before being filtered through a 0.22 μm filter. Labeled virus particles were then bound to pre-chilled cells at an MOI˜5 (by pre-titered visual inspection) at 4° C. for 15 minutes. Following binding, cells were washed three times with cold PBS before being placed at 37° C. for 40 minutes. Cells were then fixed in 4% paraformaldehyde for 20 minutes and imaged. Images were acquired on a Nikon Eclipse TE2000-E microscope equipped with a Yokogawa CSU 10 spinning-disk confocal unit and a 512-by-512 Hamamatsu 9100c-13 EM-BT camera using a 60×/1.45 numerical aperture (NA) Nikon Plan APO Apo TIRF oil immersion objective. Non-fused influenza particles appear red, as the green signal of DiOC18 (Em, 501 nm) is suppressed by a combination of self-quenching and FRET from DiOC18 to R18 (Em, 578 nm). In contrast, fused particles appear green, due to loss of FRET and self-quenching. Images were processed for Gaussian background subtraction, and then by automated particle counting for the number of red and green particles using ImageJ. The number of green particles was then divided by the number of red particles to obtain the reported ratio of fused to non-fused events for each field.

Trafficking Assays. Cells were transfected with GFP-Lamp1 (Addgene Plasmid No. 34831, deposited by Esteban Dell'Angelica, University of California at Los Angeles, Los Angeles, Calif., United States of America) using Lipofectamine 2000 (Catalog No. 11668-030; Invitrogen) and incubated overnight in growth media. The next day, PR8 influenza was incubated with 1 μM R18 for 1 hour at room temperature as described previously (Sakai et al., 2017). Labeled viruses were filtered through a 0.22 μm filter and then immediately bound to pre-chilled cells at an MOI>1 at 4° C. for 15 minutes. For EGF trafficking, Alexa Fluor 555-EGF was bound to pre-chilled cells at 4° C. for 15 minutes at a final concentration of 100 ng/mL. Following binding with either fluorescently labeled IAV or fluorescently labeled EGF, cells were washed three times with PBS followed by the addition of prewarmed media lacking IAV or EGF and placed at 37° C. for 40 minutes. Cells were then fixed in 4% paraformaldehyde containing 5 ug/mL of Hoescht 33342 (Catalog No. H3570; Thermo Fischer Scientific) for 20 minutes before imaging. Images were acquired on a Nikon Eclipse TE2000-E microscope equipped with a Yokogawa CSU 10 spinning-disk confocal unit and a 512-by-512 Hamamatsu 9100c-13 EM-BT camera. Samples were acquired using a 100×/1.45 numerical aperture (NA) Nikon Plan Apo TIRF oil immersion objective.

To quantify colocalization of IAV or EGF with Lamp1, 100 independent images per experiment from two independent experiments were captured. Each image was uniformly processed for Gaussian background subtraction and then for the Mander's coefficient of colocalization of IAV or EGF with Lamp1 using the automated JACoP plugin in ImageJ. To quantify the number of IAV or EGF particles, each image was uniformly processed for Gaussian background subtraction and then particles were counted using the automated particle analysis tool in ImageJ.

Measurement of (endo)lysosomal pH. pH of lysosomes was measured using a FITC-dextran conjugate as described previously (Yamauchi & Helenius, 2013). Cells were plated at a density of 9000 cells/cm² in 35 mm dishes and incubated in cell culture medium containing 0.1 mg/mL FITC-dextran for 72 hours. Cells were then pulsed in medium without FITC-dextran for 2 hours, lifted by trypsinization and washed with PBS. Cells were then resuspended in PBS and analyzed on a Becton Dickinson four color FACSCalibur flow cytometer by exciting with a 488 nm laser and collecting emission data at 530 (FL1) and 610 nm (FL2). The FL1/FL2 ratios of samples were compared to a standard curve generated using cells incubated with pH calibrated Britton-Robinson buffers containing 50 mM sodium azide, 50 mM 2-deoxyglucose, and 10 μM nigericin.

### Example 1

### Glucosylceramide Metabolism Regulates Influenza Infection

Previous studies have demonstrated that certain enzymes along the sphingolipid pathway (FIG. 1) might be important for influenza infection; cells exposed to inhibitors of serine palmitoyltransferase or sphingosine kinase, as well as cells deficient in sphingomyelin synthase, displayed reduced influenza infection (Seo et al., 2010; Seo et al., 2013; Tafesse et al., 2013). However, the roles of glucosylceramidase (GBA) or its substrate, glucosylceramide (GlcCer), have not been explored in influenza infections.

To examine the role of GBA in influenza infection, knockout (KO) cell lines lacking GBA were generated using clustered regularly interspaced short palindromic repeats with Cas9 (CRISPR/Cas9) gene editing. CRISPR/Cas9 was performed in two cell lines, human embryonic kidney (HEK) 293 and adenocarcinomic lung epithelial A549. HEK 293 cells were chosen for their ease of and broad use in cell biology, while A549 cells were chosen as they, being derived from lung epithelia, are considered more physiologically relevant for influenza research.

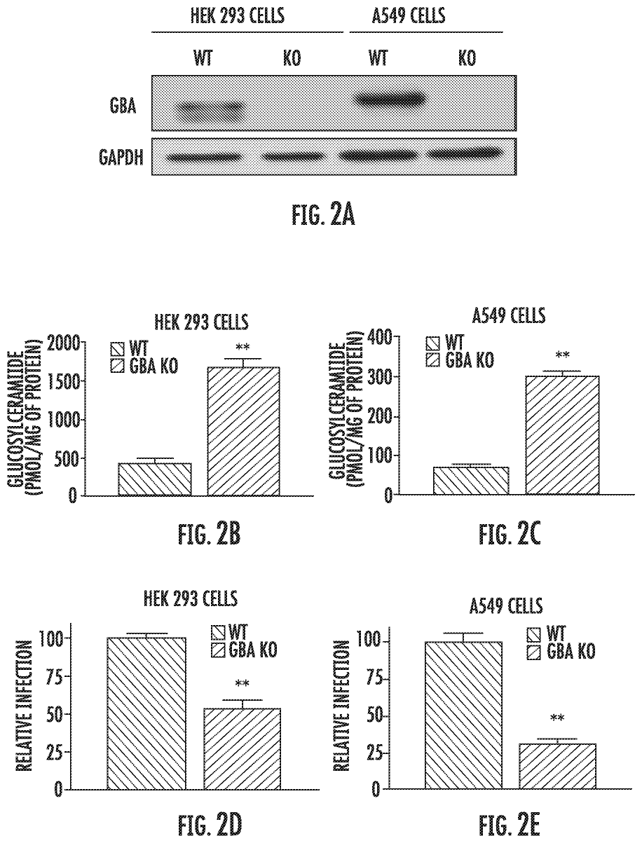

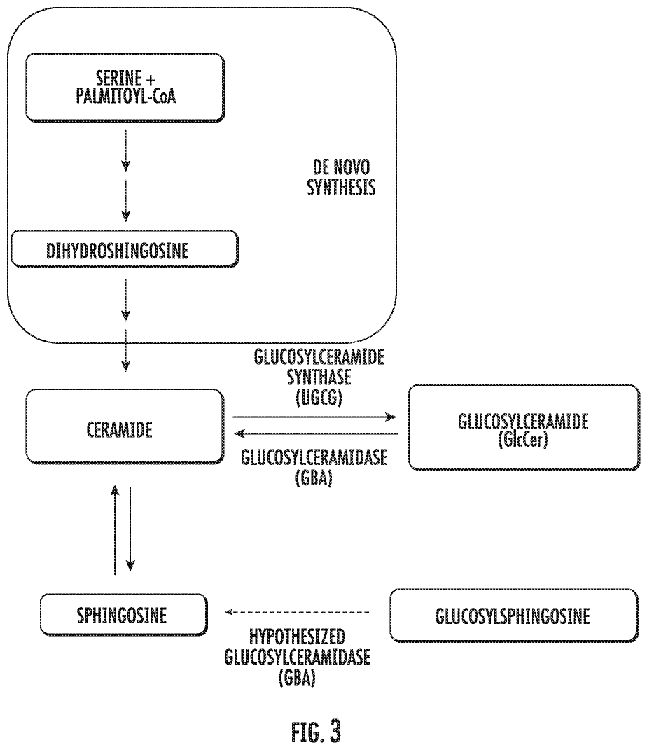

After preforming CRISPR/Cas9 gene editing using a gRNA used to target GBA having the sequence 5′-AAGCTTCGGCTACAGCTCGG-3′ (SEQ ID NO: 15) and isolating individual clones, cells were analyzed for expression of GBA by western blot analysis and for the resulting concentration of GlcCer by mass spectrometry. In both HEK 293 and A549 cells CRISPR/Cas9 targeting resulted in complete loss of GBA protein as detected by western blotting (FIG. 2A). Mass spectrometry revealed that in both 139 HEK 293 and A549 cells, GBA KO resulted in ˜3-4 fold increase in GlcCer levels (FIGS. 2B and 2C).

After confirming functional KO of GBA in the two cell lines described above, the effect of loss of GBA on influenza infection was examined. As seen in FIGS. 2D and 2E, GBA KO in HEK 293 cells resulted in ˜50% decrease in PR8 influenza infection, while in A549 cells GBA KO resulted in ˜70% decrease.

### Example 2

### Changes in Sphingolipid Species in GBA KO Cells

As noted above, loss of GBA resulted in an expected increase in GlcCer levels. Sphingomyelin, ceramide, glucosylceramide, glucosylsphingosine, sphingosine, and sphingosine-1-phosphate were analyzed by mass spectrometry in all knockout cells and compared to their WT counterparts. Mass spectrometry peaks were compared to internal standards and all data are represented as pmol lipid/mg of protein (mean values shown, n=6). The data for GlcCer are the same as those displayed graphically in FIGS. 2B and 2C. Table 4 below presents the mass spectrometry data. Surprisingly, this was not accompanied by a corresponding decrease in ceramide levels (see Table 3 below). Moreover, major changes in downstream ceramide metabolites such as sphingosine-1-phosphate and sphingomyelin were not noted. Taken together, these data are consistent with the lack of change in ceramide levels being due to compensatory de novo synthesis of ceramide, which was also consistent with the increase seen in dihydrosphingosine (Table 3), an intermediate metabolite in de novo synthesis (see FIG. 1 and FIG. 3, shaded). Interestingly, GBA KO cells displayed an even greater fold change in glucosylsphingosine (Table 3), although the total mass of glucosylsphingosine is hundreds fold lower than that of glucosylceramide. Little is known regarding the metabolism of glucosylsphingosine, but its increase suggests a potential new role for GBA in the catabolism of glucosylsphingosine (FIG. 3).

TABLE 3
Sphingolipid Species Regulated by Glucosylceramidase

| | HEK 293 Cells | | | A549 Cells | | |
|---|---|---|---|---|---|---|
| | (pmol/mg of protein) | | | (pmol/mg of protein) | | |
| | | | Fold | | | Fold |
| Species* | WT | GBA KO | Change | WT | GBA KO | Change |
| Glucosylceramide | 430.00 | 1664.39 | 3.877 | 71.33 | 333.34 | 4.67 |
| Glucosylsphingosine | 0.70 | 55.50 | 79.29 | 0.16 | 1.67 | 10.44 |
| Dihydrosphingosine | 5.15 | 13.43 | 2.61 | 12.19 | 25.79 | 2.12 |
| Dihydrosphingosine-1-PO₄ | 0.90 | 1.15 | 1.28 | 0.71 | 0.99 | 1.39 |
| Ceramide | 790.87 | 1008.08 | 1.27 | 87.46 | 186.55 | 2.13 |
| Sphingosine | 113.22 | 134.92 | 1.19 | 86.26 | 115.10 | 1.33 |
| Sphingomyelin | 13011.30 | 14909.43 | 1.15 | 6770.50 | 5284.64 | 0.78 |
| Sphingosine-1-PO₄ | 1.84 | 1.60 | 0.87 | 0.51 | 1.45 | 2.84 |

### Example 3

### Glucosylceramidase Regulates Entry of Influenza and Other Endocytosed Viruses

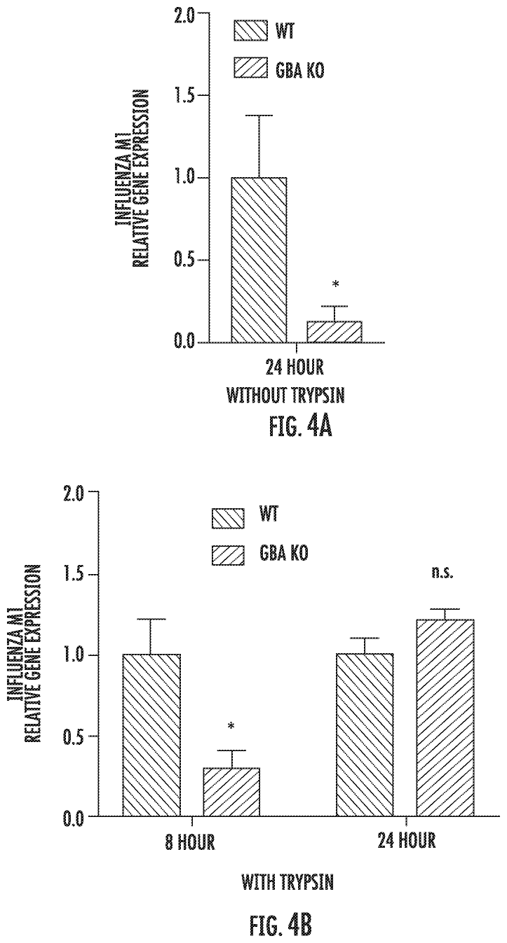

The reduction in influenza infection observed in GBA KO cells (FIGS. 2D and 2E) could be due to defects at different stages of the viral life cycle. To begin to identify the step requiring GBA, IAV Matrix Protein 1 (M1) mRNA was analyzed after 24 hours in cells incubated in media lacking trypsin, in order to limit IAV infections to one cycle of replication. GBA KO cells exhibited reduced IAV M1 expression after 24 hours (FIG. 4A). IAV M1 mRNA was then analyzed at two timepoints post infection in the presence of trypsin (to cleave the HA precursor and therefore permit production of infectious particles). After 8 hours, GBA KO cells displayed reduced IAV M1 expression compared to WT cells, but no difference was seen at 24 hours (FIG. 4B). These data suggested that the observed reduction in influenza infection seen in FIGS. 2D and 2E was limited to one cycle of replication, likely at the level of virus entry.

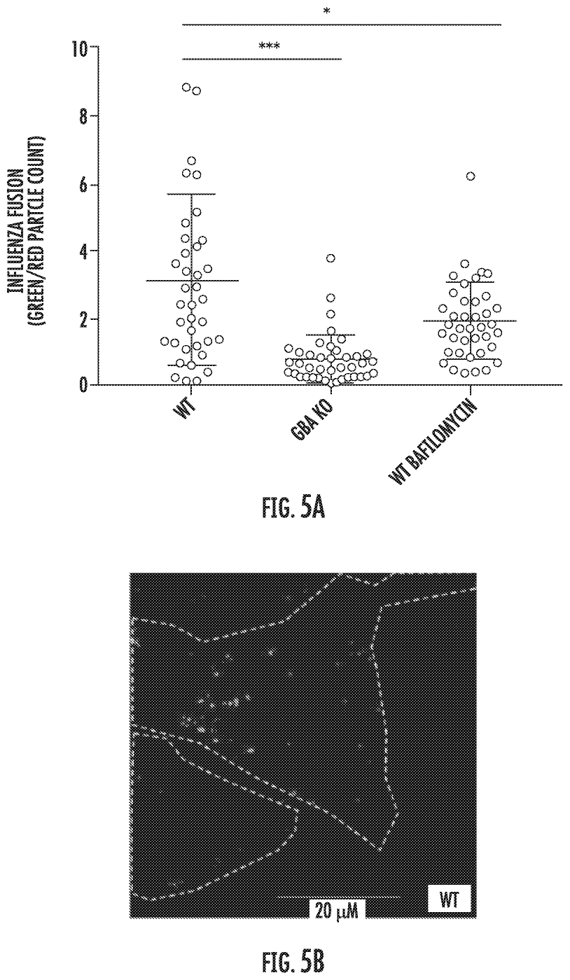

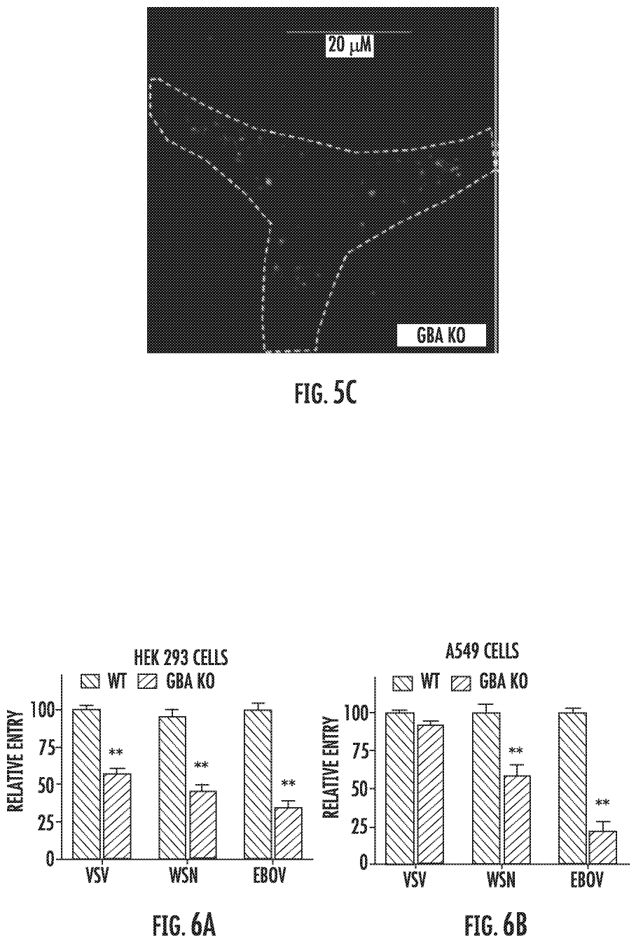

To test whether GBA regulates influenza virus entry (through endosomes) fusion of A/PR/8/34 influenza (H1N1) labeled with octadecyl rhodamine B chloride (R18) and 3,3′-dioctadecyloxacarbocyanine (DiOC18) was monitored. Fusion results in a shift in fluorescence emission from red (R18, 586 nm) to green (DiOC18, 510 nm) due to separation of the probes upon fusion and dilution into the endosome membrane (Sakai et al., 2006; Banerjee et al., 2013). As seen in FIG. 5A, in WT cells there were ˜3.5 fused influenza particles for every non-fused particle, while in the GBA KO cells the corresponding ratio was less than 1. The results in FIG. 5 indicated that GBA was necessary for influenza particles to fuse in endosomes and suggested that the observed reduction in influenza infection (FIGS. 2D, 2E, 4A, and 4B) was due to a defect in the entry phase of the viral life cycle.

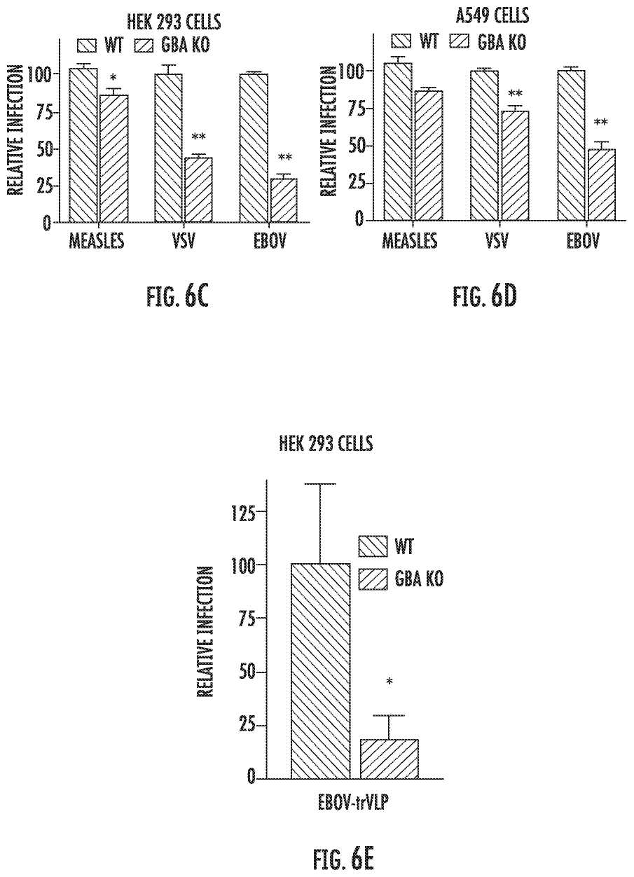

Whether entry of other endocytosed enveloped viruses could be impacted by loss of GBA was also examined. To do this, virus-like particles (VLPs) with an influenza Matrix-1 (M1)-β-lactamase (β-lam) core and bearing different viral glycoproteins on their surfaces, VSV G, which directs fusion in early endosomes (pH ˜6.0), WSN influenza HA, which directs fusion in late endosomes (pH ˜5.0-5.5), and Ebola GP, which direct fusion in endolysosomes (pH ˜4.5-5.0; see Bär et al., 2006; Libersou et al., 2010; Banerjee et al., 2013), were generated. WT and GBA KO cells were incubated with the VLPs and assayed for VLP entry using a fluorescent R-lam substrate in conjunction with flow cytometry (FIGS. 6A and 6B). Entry mediated by VSV-G was reduced in HEK 293 GBA KO cells, but unaffected in the corresponding A549 KO cells. Entry mediated by the HA of WSN influenza was reduced in both the HEK 293 and A549 GBA KO cells, consistent with the PR8 infection (FIGS. 2D, 2E, 4A, and 4B) and fusion (FIGS. 5A-5C) data. Entry mediated by EBOV-GP was also reduced in both KO cell lines, and trended towards a lower extent of entry than entry mediated by the glycoproteins of WSN influenza or VSV. These findings suggested that other viruses that enter cells through endosomes depended on GBA and were consistent with a greater entry inhibition for viruses that fuse with later endosomes, suggesting that GBA might affect endosome maturation and/or acidification. The differing results for VSV-G201 between A549 and HEK 293 GBA KO cells might indicate a cell type dependence for GBA in the early endocytic pathway.

To further explore the breadth of viruses whose entry is affected by GBA, pseudoviruses with a VSV core and displaying the glycoproteins of measles virus, which fuses at neutral pH at the cell surface or the glycoproteins of the VSV and EBOV, which enter cells through endosomes, were employed. Infections by VSV pseudoviruses bearing the VSV and EBOV glycoproteins were decreased in GBA KO cells (FIGS. 6C and 6D), as seen with their corresponding influenza M1-VLPs (FIGS. 6A and 6B). In contrast, infection by pseudoviruses bearing the glycoproteins of measles virus appeared less dependent on GBA, consistent with the contention that virus entry through endosomes, particularly through late endosomes, was dependent on functional GBA and by extension, optimal levels of GlcCer.

Interestingly, the role of GBA in EBOV infections was tested using EBOV transcription-replication competent VLPs (trVLPs), which recapitulate the full Ebola lifecycle and can be used under BSL2 conditions (Hoenen et al., 2014). As seen in FIG. 6E, loss of GBA strongly reduced Ebola trVLP infection even after multiple cycles of replication. These trVLP data contrasted with the finding in FIGS. 4A and 4B that the effect of GBA on influenza infection was limited to viral entry. Taken together, FIG. 4A, FIG. 4B, and FIG. 6E suggested that the role of GlcCer in viral assembly and/or budding could be virus-specific.

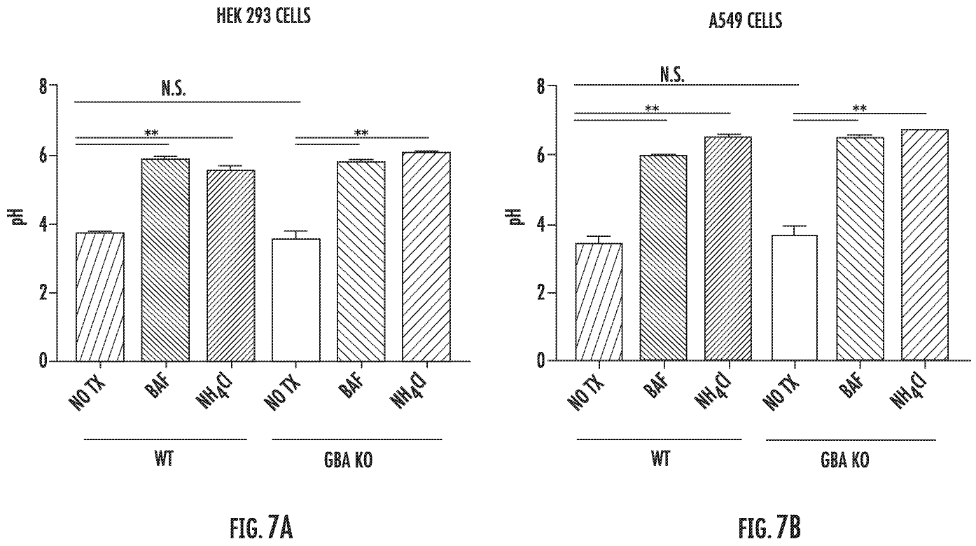

As the observed decreases in entry by viruses that fuse in late endosomes could be due to a defect in endosome acidification, the pH of endosomes in GBA KO cells was measured. To accomplish this, a dual-emission ratiometric technique was employed after feeding cells FITC-dextran (Eriksson et al., 2017). Two independent positive controls (Bafilomycin and NH4Cl) were used to confirm changes in pH. Using this technique, it was determined that the pH in endosomes in GBA KO cells was not detectably different than that in WT cells (FIGS. 7A and 7B). These data suggested that the observed viral entry phenotype was not due to changes in the acidification process of the endosome.

### Example 4

### Glucosylceramidase Regulates Influenza Trafficking Along the Endocytic Pathway

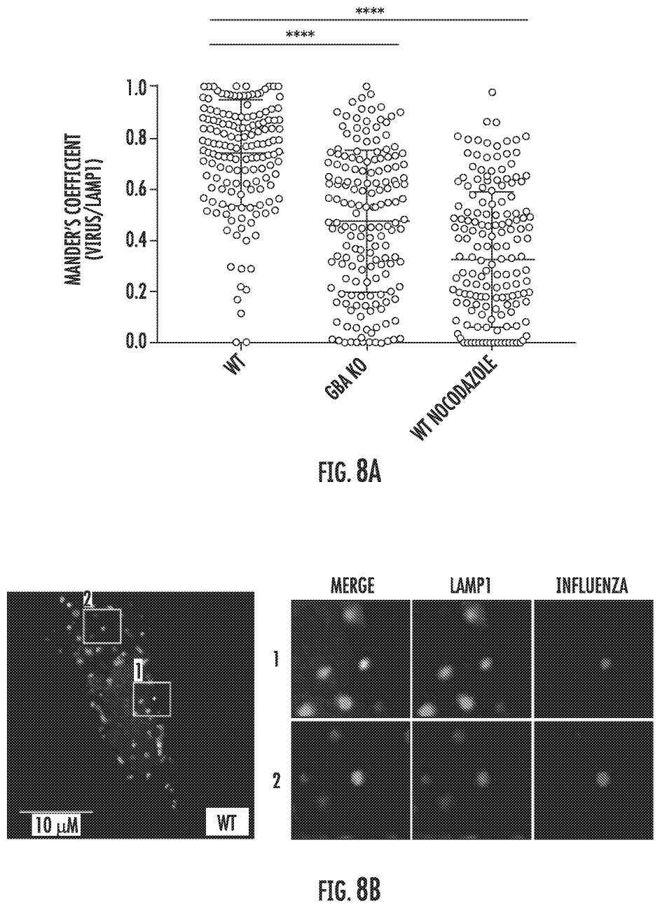

It is well established that influenza virus traffics to late endosomes for fusion (White et al., 1982). In addition, GlcCer has been implicated in lipid transport along the endocytic pathway (Sillence, 2002). Whether GBA was important for trafficking of influenza particles to late endosomes was thus examined. WT and GBA KO A549 cells were transfected with Lamp1-GFP and then the cells were infected with R18-labeled influenza virus (red). In WT cells at 40 minutes post-warming, influenza was visualized in Lamp1+(green) endosomes in a high percentage of cells (FIGS. 8A and 8B). In contrast, colocalization was reduced in GBA KO cells (FIGS. 8A and 8C), albeit not as strongly as in WT cells treated with nocodazole (FIG. 8A), a microtubule inhibitor known to block trafficking between early and late endosomes. The observation that equivalent numbers of influenza particles were seen in WT and GBA KO cells (FIG. 8D) indicated that there was not a defect in influenza binding to GBA KO cells, but rather a defect in trafficking along the endocytic pathway.

### Example 5

### Glucosylceramidase Regulates EGFR Trafficking Along the Endocytic Pathway

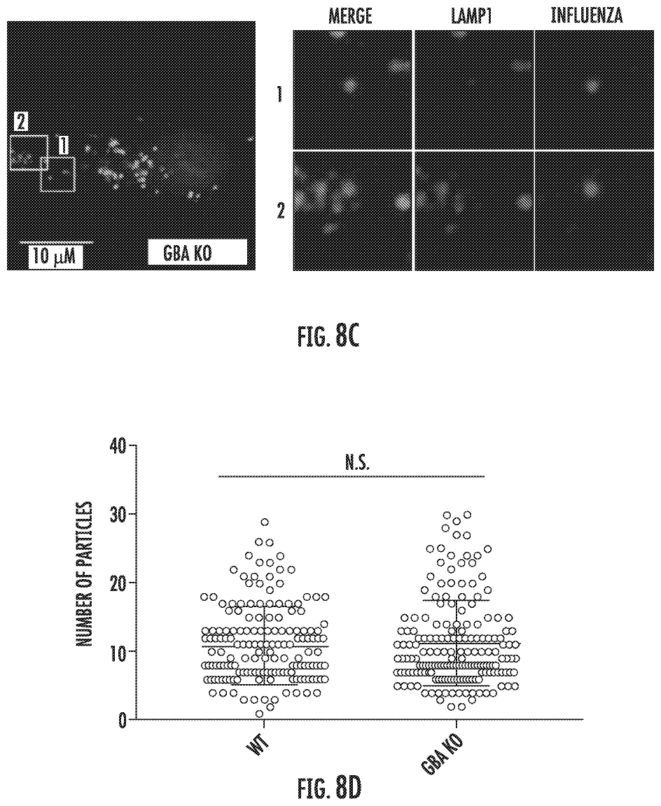

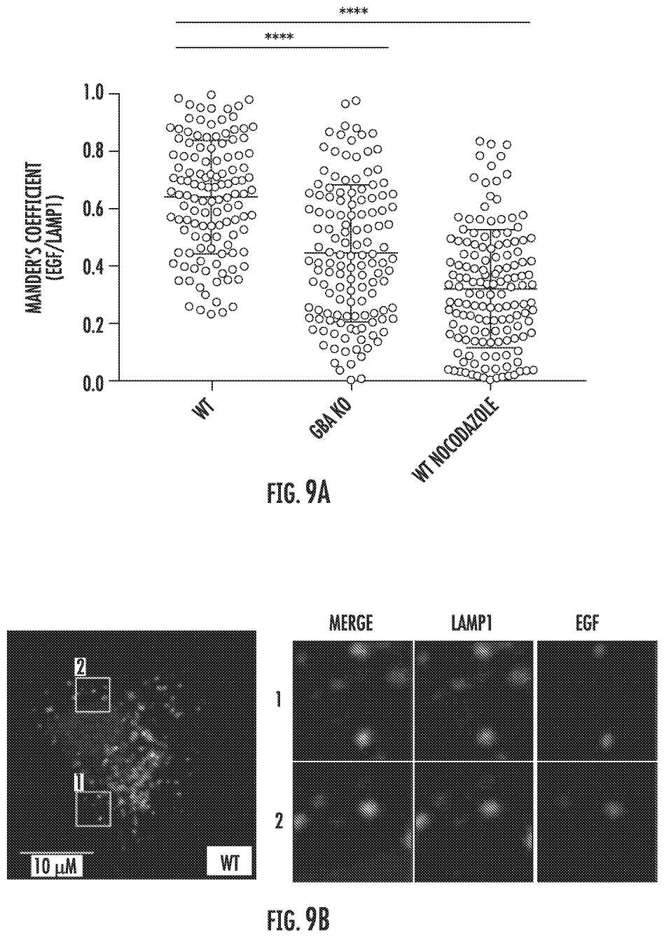

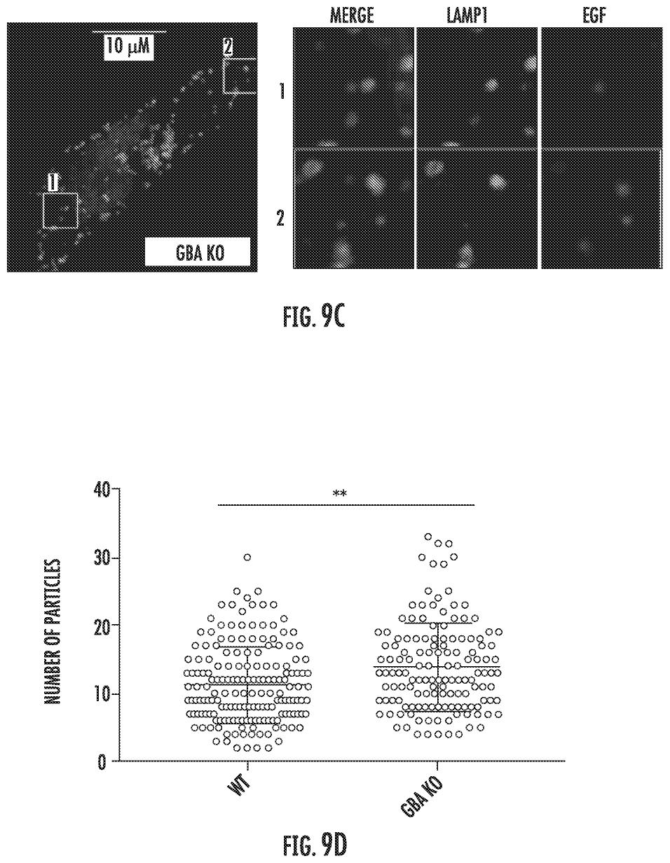

In view of the findings on influenza trafficking, whether GBA was required for proper trafficking of non-viral cargo along the endocytic pathway was tested. The same approach as in FIGS. 8A-8D was utilized, but with fluorescently tagged epidermal growth factor (EGF; red; FIGS. 9A-9D). Similar to influenza, in WT cells at 40 minutes post-warming, EGF was seen in a high percentage of Lamp1+(green) endosomes, while colocalization was reduced in GBA KO cells. As for influenza, the decrease in colocalization of EGF with Lamp1 was not as severe as seen in WT cells treated with nocodazole (FIG. 9A). There was no decrease in the number of EGF particles in GBA KO vs. WT cells 253 (FIG. 9D), in fact a small increase was seen, indicating a post-binding requirement for GBA for proper trafficking of EGF.

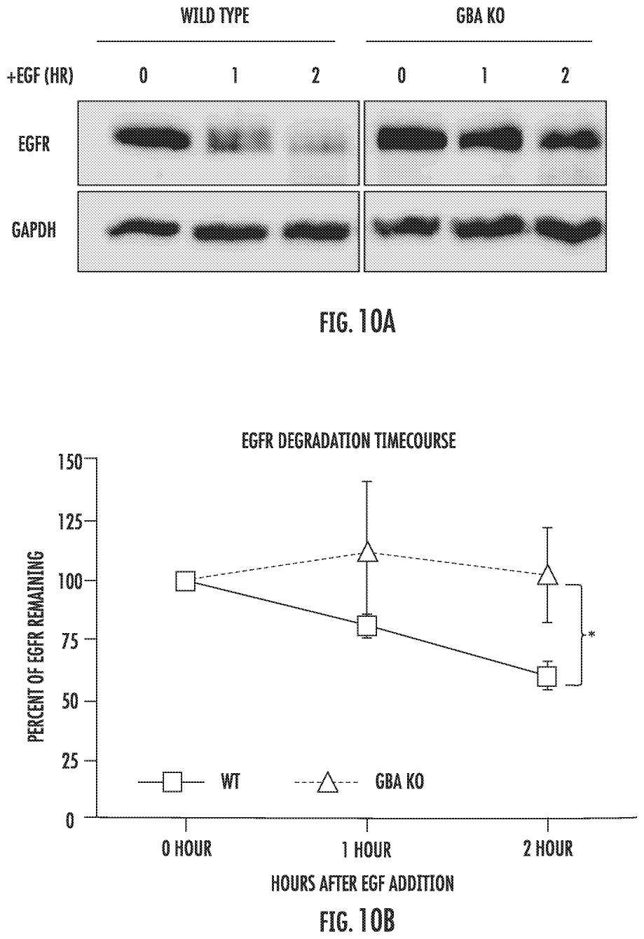

Following binding of EGF to its receptor (EGFR) at the cell surface, the EGF-EGFR complex is transported to lysosomes and degraded by proteases including cathepsins (Opresko et al., 1995). Since EGF trafficking was impaired in GBA KO cells, whether degradation of EGFR was also impaired was determined. Media containing EGF was added to cells at 37° C., and at various times the cells were harvested, lysed, and analyzed for the presence of EGFR by Western blot analysis. As seen and quantified in FIGS. 10A and 10B, and consistent with the EGF trafficking data of FIGS. 9A-9D, degradation of EGFR was notably impaired in GBA KO cells.

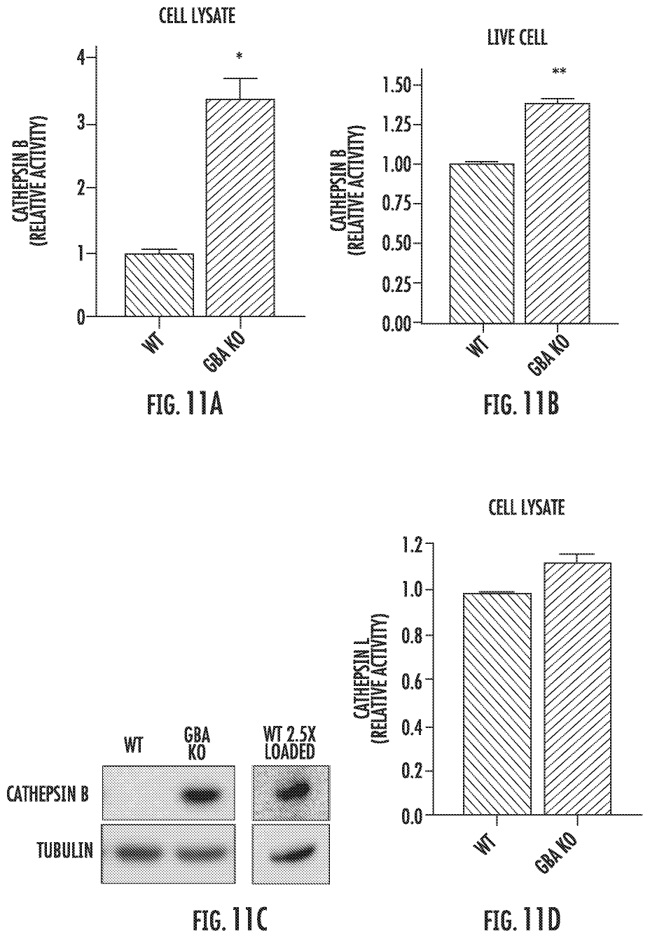

It is possible that the defect in EGFR degradation in GBA KO cells (FIGS. 10A and 10B) was due to a defect in EGFR trafficking (FIGS. 9A-9D) as opposed to a defect in endosome acidification (FIGS. 7A and 7B) or cathepsin activities. To test the latter possibility, the levels of two cysteine proteases normally found in lysosomes, Cathepsin B (CatB) and CatL, were examined. This was done in vitro following cell lysis and adjustment to an acidic pH for CatB and CatL using fluorescent peptide substrates, and, additionally for Cat B, in live cells using MAGIC RED™ (RR)₂ brand reagent (Bio-Rad Laboratories, Inc., Hercules, Calif., United States of America; see also Duncan et al., 2009). Unexpectedly, GBA KO cells displayed an ˜3.5-fold increase in CatB activity as compared to WT cells in the in vitro assay (FIG. 11A). CatB activity was also increased in the live cell assay, but not to as great an extent (FIG. 11B). Consistently, an increase in CatB protein levels was observed in GBA KO cells (FIG. 11C). CatL activity in cell lysates displayed no significant difference in KO as compared to WT cells (FIG. 11D).

Taken together, these data suggested that diminished cathepsin expression and/or activity were not the cause of dysfunctional EGFR degradation in GBA KO cells, and further supported a mechanism involving impaired endosome trafficking. The mechanism behind the unexpected increase in CatB activity and expression in GBA KO cells could be a result of defects in CatB localization.

### Example 6

### Glucosylceramide Synthase Regulates Influenza Virus Infection

As set forth in more detail herein above, the loss of expression or inhibition of several sphingolipid-metabolizing enzymes leads to reduced influenza virus infection (FIG. 12), and glucosylceramidase (GBA) was required for optimal influenza virus entry (see also Drews et al., 2019). Glucosylceramide synthase (UGCG), which converts ceramide to glucosylceramide, is another enzyme in the same biochemical pathway, and thus might also play a role in the influenza virus life cycle (green box).

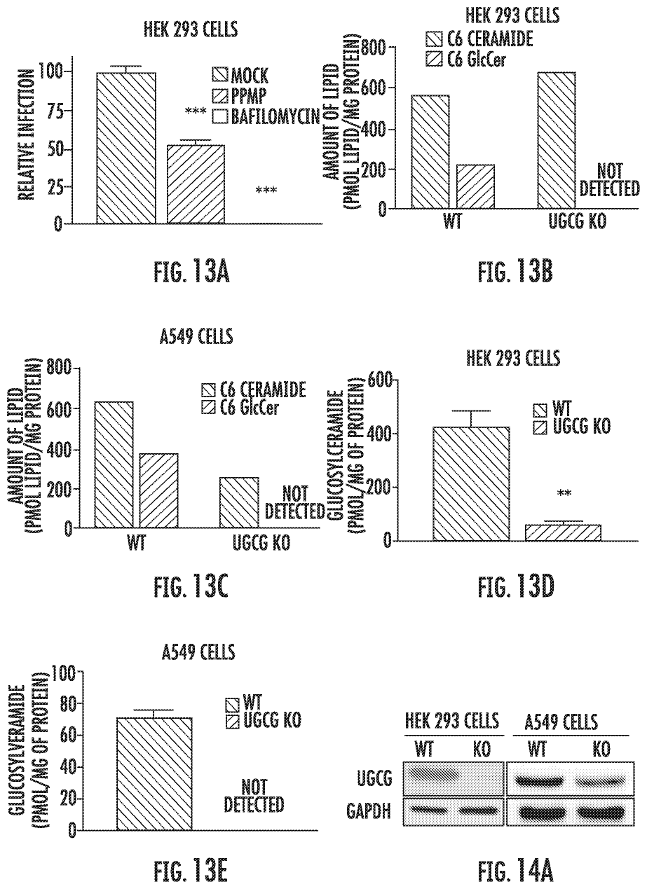

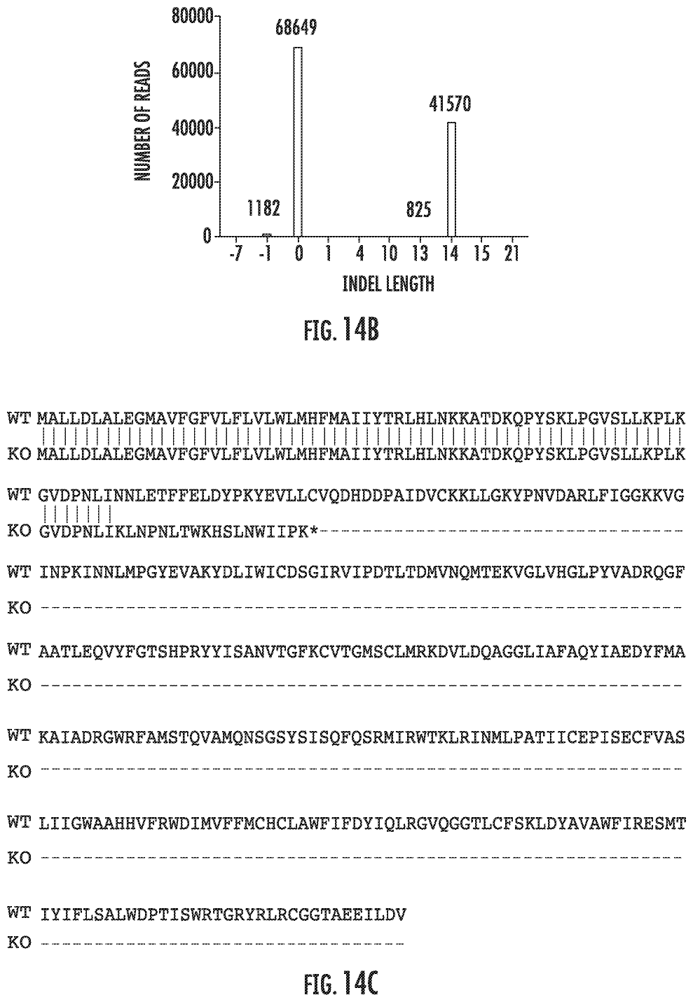

To determine whether UGCG inhibition could impact influenza infection, target cells were treated with DL-threo-1-phenyl-2-palmitoylamino-3-morpholino-1-propanol (PPMP), a broadly utilized inhibitor of UGCG (Grazide et al., 2004; Turikovi et al., 2014; Wegner et al., 2018). A decrease in infection by PR8 influenza virus encoding NS1-GFP was observed as monitored by flow cytometry for GFP expression (FIG. 13A). However, to ensure these results were not due to any off target effects of PPMP, a genetic knockout of UGCG was also generated.

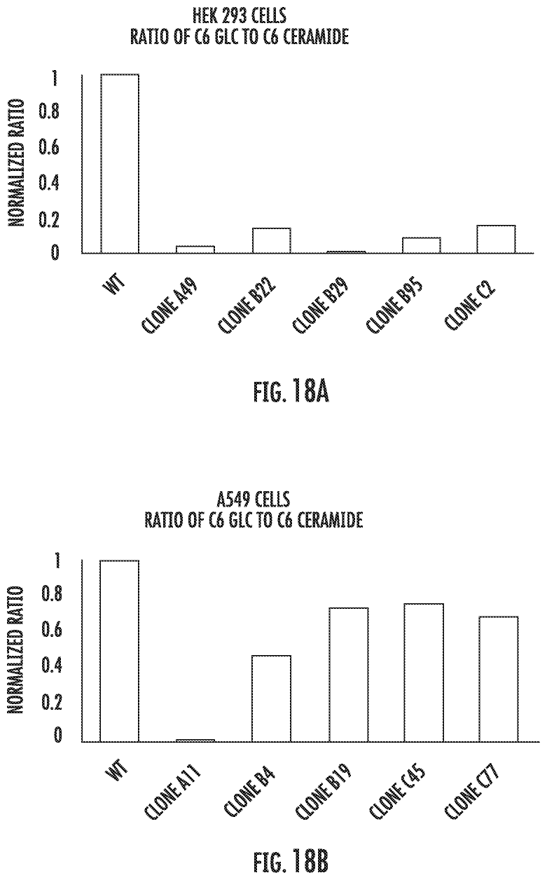

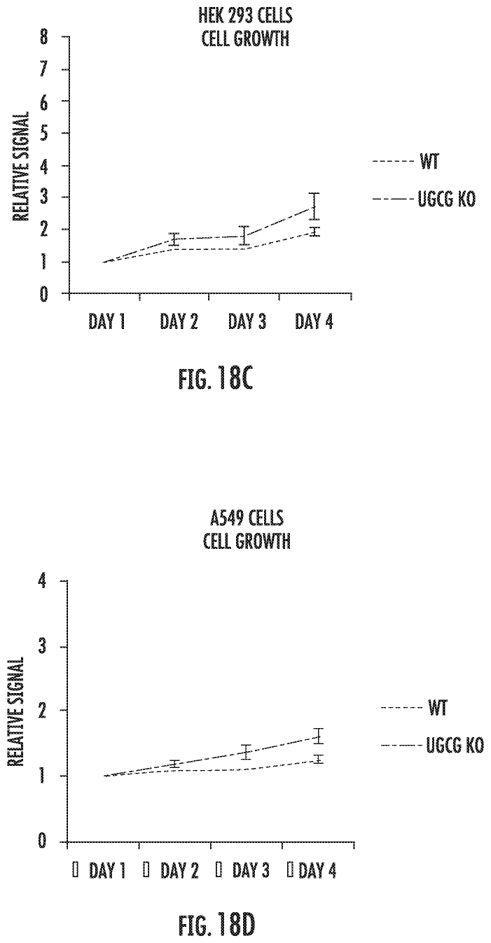

The CRISPR/Cas9 system was employed to knockout UGCG (the sgRNA targeting UGCG had the nucleotide sequence 5′-TCCTAACTTAATCAACAACC-3′; SEQ ID NO: 16) in HEK 293 and A549 cells, and the functional status of UGCG in putative knockout lines was determined. HEK 293 cells were chosen for their ease of transfection and A549 cells were selected as a more physiologically relevant in vitro system for influenza virus research, as they were derived from human lung cells (and influenza virus is a respiratory pathogen). Potential UGCG KO clones were screened by assaying for UGCG enzyme activity by incubating cells with C6-ceramide, a synthetic short-chain ceramide (see FIGS. 18A and 18B). Wild-type cells containing functional UGCG convert C6-ceramide to C6-GlcCer. However, in both HEK 293 and A549 UGCG KO cells, conversion of C6-ceramide to C6-GlcCer was not seen, indicating a full ablation of UGCG functional activity (FIGS. 13B and 13C).

Next, the endogenous basal (i.e., in uninfected cells) levels of GlcCer were measured in WT and the chosen HEK293 and A549 KO cells. Full basal sphingolipid profiles from UGCG knockout cells. Sphingosine, glucosylceramide, sphingomyelin, sphingosine-1-phosphate, and ceramide in uninfected KO and WT cells were analyzed by liquid chromatography-mass spectrometry. It was determined that HEK 293 UGCG KO cells displayed significantly decreased GlcCer levels, and GlcCer was undetectable in A549 UGCG KOs (FIGS. 13D and 13E). While, as expected, both cell types displayed reduced levels of GlcCer, ceramide levels were not correspondingly elevated (see Table 4), which might have resulted from “shunting” of ceramide to other downstream metabolites. Interestingly, ablation of UGCG activity in HEK 293 and A549 cells did not result in the same changes in downstream sphingolipid metabolic species between the two cell lines, as HEK 293 cells showed an elevation in sphingosine-1-phosphate, while A549 cells displayed elevations in sphingomyelin (described in more detail herein below in the Discussion of the EXAMPLES section). A full list of sphingolipid species regulated by glucosylceramide synthase can be found in Table 4.

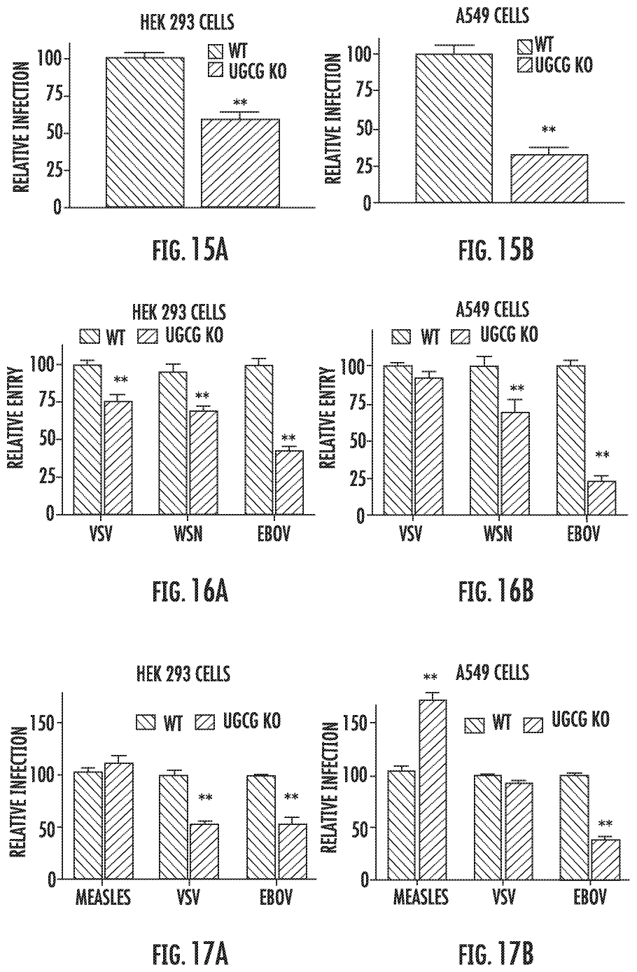

Finally, to determine if knockouts of UGCG affected influenza virus infection, the GFP-encoding PR8 influenza virus as in FIG. 13A was employed. As seen in FIGS. 15A and 15B, influenza virus infection levels were decreased in both HEK 293 and A549 UGCG KO cells compared to WT cells. Together, these data demonstrated that pharmacological and molecular disruption of cellular GlcCer synthesis led to suppression of influenza virus infection.

TABLE 4
Sphingolipid Species Regulated by Glucosylceramide Synthase

| | HEK 293 Cells | | | A549 Cells | | |
|---|---|---|---|---|---|---|
| | | UGCG | Fold | | UGCG | Fold |
| Species* | WT | KO | Change | WT | KO | Change |
| GlcCer C16 | 71.11 | 24.98 | 0.35 | 28.56 | N.D. | N.D. |
| GlcCer C18 | 7.87 | 1.86 | 0.24 | 2.72 | N.D. | N.D. |
| GlcCer C20 | 17.69 | 1.23 | 0.07 | 0.64 | N.D. | N.D. |
| GlcCer C22 | 83.96 | 7.28 | 0.09 | 2.91 | N.D. | N.D. |
| GlcCer C22:1 | 18.64 | 10.01 | 0.05 | 1.46 | N.D. | N.D. |
| GlcCer C24 | 78.06 | 11.25 | 0.14 | 2.41 | N.D. | N.D. |
| GlcCer C24:1 | 147.36 | 17.86 | 0.12 | 21.85 | N.D. | N.D. |
| GlcCer C26 | 0.72 | 0.17 | 0.24 | N.D. | N.D. | N.D. |
| GlcCer C26:1 | 4.66 | 0.66 | 0.14 | 0.58 | N.D. | N.D. |
| GlcCer Totals | 759.40 | 66.31 | 0.09 | 73.38 | N.D. | N.D. |
| Ceramide C16 | 32.70 | 40.94 | 1.25 | 33.44 | 34.66 | 1.04 |
| Ceramide C18 | 37.19 | 37.80 | 1.02 | 3.61 | 4.28 | 1.25 |
| Ceramide C20 | 17.67 | 6.81 | 0.39 | 0.34 | 0.45 | 1.35 |
| Ceramide C22 | 115.50 | 54.39 | 0.47 | 1.15 | 1.76 | 1.53 |
| Ceramide C22:1 | 15.45 | 7.22 | 0.47 | 0.89 | 2.54 | 2.85 |
| Ceramide C24 | 155.91 | 146.76 | 0.94 | 220 | 3.13 | 1.43 |
| Ceramide C24:1 | 384.97 | 194.61 | 0.51 | 31.94 | 59.77 | 1.87 |
| Ceramide C26 | 2.62 | 3.37 | 1.29 | 0.26 | 0.56 | 2.15 |
| Ceramide C26:1 | 13.50 | 7.38 | 0.55 | 0.24 | 0.96 | 4.30 |
| Ceramide Totals | 775.52 | 499.25 | 0.64 | 73.88 | 108.12 | 1.46 |
| Sphing C16 | 8589.64 | 6251.15 | 0.73 | 4164.63 | 8087.13 | 1.94 |
| Sphing C18 | 1209.58 | 1196.49 | 0.99 | 341.60 | 675.41 | 1.98 |
| Sphing C20 | 1055.36 | 541.38 | 0.51 | 115.96 | 188.50 | 1.63 |
| Sphing C22 | 817.54 | 802.26 | 0.98 | 218.37 | 368.16 | 1.69 |
| Sphing C22:1 | 259.74 | 134.07 | 0.52 | 53.99 | 209.19 | 3.87 |
| Sphing C24 | 382.17 | 625.26 | 1.64 | 144.76 | 312.34 | 1.16 |
| Sphing C24:1 | 2011.61 | 1648.51 | 0.82 | 881.30 | 3877.56 | 4.40 |
| Sphing C26 | 6.68 | 13.87 | 2.08 | 6.99 | 11.47 | 1.64 |
| Sphing C26:1 | 31.69 | 45.24 | 1.43 | 15.18 | 68.46 | 4.51 |
| Sphing Totals | 14364.01 | 11256.24 | 0.78 | 5942.78 | 13798.25 | 2.32 |
| Sphingosine | 111.50 | 36.27 | 0.33 | 86.26 | 166.19 | 1.93 |
| Dihydrosphingosine | 5.09 | 1.81 | 0.36 | 12.19 | 38.35 | 3.15 |
| Sphingosine-1-PO4 | 1.70 | 25.08 | 14.75 | 0.51 | 0.99 | 1.94 |
| Dihydrosphingosine-1-PO₄ | 0.88 | 1.36 | 1.55 | 0.75 | 1.31 | 1.75 |
| Hexosylsphingosine | 0.67 | 0.41 | 0.61 | 0.19 | 0.29 | 1.53 |

GlcCer: Glucosvlceramide;
Sphing: Sphingomyelin;
N.D.: not determined
*The data represent the averages from five biological replicates and are represented as pmol lipid/mg of protein.

### Example 7

### Glucosylceramide Synthase Regulates Entry of Influenza Virus and Other Endocytosed Viruses

To test whether the reduction in influenza virus infection in UGCG KO cells was due to a reduction in influenza virus entry into the cells, virus-like particles (VLPs) containing an influenza virus Matrix-1 (M1)-β-lactamase (β-lam) core and a membrane bearing the HA and NA glycoproteins of WSN influenza virus, which fuses with host endosomes at pH ˜5.9-6.0 (Marvin et al., 2017) were generated. The analysis was extended to include influenza virus Matrix-1 VLPs displaying the glycoproteins of vesicular stomatitis virus (VSV), which fuses with host early endosomes (pH ˜6.0), and Ebola virus (EBOV), which fuses with host endolysosomes (pH ˜4.5-5.5; Bär et al., 206; Libersou et al., 2010; Banerjee et al., 2013). It was determined that both WSN influenza virus HA/NA and EBOV GP Matrix-I VLPs displayed reduced entry into both HEK 293 and A549 UGCG KO cells compared to WT cells. VSV G VLPs displayed reduced entry into HEK 293 UGCG KOs, but not into A549 UGCG KOs (FIGS. 16A and 16B), which might have been due to the different tissue origins of these cells.

To further explore the extent to which UGCG mediates viral infections, a VSV pseudovirus system was employed, and how pseudoviruses bearing the glycoproteins of VSV, EBOV, and measles (a virus that employs its H and F proteins to fuse at the plasma membrane) infected UGCG KO and WT cells was determined. As seen in FIGS. 17A and 17B, and similar to the results seen with influenza virus Matrix-1 VLPs (FIGS. 16A and 16B), VSV G-mediated pseudovirus infection was reduced in HEK 293 UGCG KOs, but unaffected in A549 UGCG Kos, whereas EBOV GP pseudovirus infection was reduced in both UGCG KO cell lines tested. In contrast, measles H/F-mediated pseudovirus infection was not inhibited in HEK 293 UGCG KOs, and was even increased in A549 UGCG KO compared to WT cells. These results indicated a role for UGCG in influenza virus entry, and suggested a similar function in the entry of other endosome-entering viruses, particularly ones that enter late in the endocytic pathway.

### Discussion of the Examples

Influenza is a viral pathogen responsible for the second largest pandemic in human history. A better understanding of how influenza enters host cells may lead to more efficacious therapies against emerging strains of the virus. The presently disclosed subject matter demonstrated that the glycosphingolipid metabolizing enzyme glucosylceramidase was required for optimal influenza trafficking to late endosomes and consequent fusion, entry, and infection. Evidence is also provided that promotion of influenza entry by glucosylceramidase extended to other endosome-entering viruses and was due to a general requirement for this enzyme, and hence optimal levels of glucosylceramide, for efficient trafficking of endogenous cargos such as the EGF receptor, along the endocytic pathway. Accordingly, the data presented herein are relevant to the basic process of endocytosis as well as pathogenic processes including virus entry and Gaucher disease.

As disclosed herein, a role for glucosylceramidase (GBA) in the regulation of endocytosis of viral and cellular cargos was established, firstly for endosomal entry and infection by influenza virus. That the defect in influenza infection in GBA KO cells was due to defects in delivery to late endosomes and consequent fusion and entry into the cytoplasm was also demonstrated. Consistently, defects in entry mediated by the glycoproteins of other enveloped viruses that enter cells through endosomes were observed (White & Whittaker, 2016); VLPs and pseudoviruses bearing VSV, influenza, and Ebola (EBOV) glycoproteins displayed decreased entry into GBA KO cells, with minimal effects seen on entry mediated by the glycoproteins from a virus (measles) that fuses and enters the cell through the plasma membrane. Interestingly, the trend was for greater reliance on functional GBA for viruses that enter through later, more acidic, endosomes (Marsh & Helenius, 2006; Grove & Marsh, 2011; Lozach et al., 2011; Yamauchi & Helenius, 2013; White & Whittaker, 2016). Loss of GBA was noted to lead to inhibition in single but not multi-cycle influenza infections (FIGS. 4A and 4B), suggesting that the perturbations to the sphingolipid pathway in our GBA KO cells might have consequences for IAV assembly or exit, findings that require further investigation. Indeed, a recent study demonstrated that the membrane of IAV might be enriched in sphingolipids, indicating a role for these lipids in the exit of the virus from host cells (Gerl et al., 2012).

Based on these findings, GBA and optimal levels of GlcCer could be required, in general, for proper trafficking of cargo along the endocytic pathway, particularly to late endosomes. Indeed, it was found that trafficking of not only influenza particles but also EGF and its receptor (to Lamp1V endosomes) was impaired in GBA KO cells, which in the latter case correlated with significantly delayed degradation of EGFR. Collectively, the presently disclosed data strongly suggested that GBA regulated normal trafficking of cargo along the endocytic pathway.

Altering the function of one enzyme in the sphingolipid pathway can results in compensation that makes it challenging to pinpoint a single lipid species as the cause of a particular cellular phenotype (Pewzner-Jung et al., 2010). It was determined, by mass spectrometry of lipids, that removal of GBA in both HEK and A549 cells resulted in a significant increase in GlcCer mass. As such, it could be that excess GlcCer and not changes in other sphingolipids was responsible for the observed phenotypes. A decrease in ceramide levels was not observed, nor were major changes to non-glycosylated ceramide metabolites, such as sphingomyelin and sphingosine-1-phosphate (FIG. 3; see also Table 4), possibly due to a compensatory increase in de novo synthesis of ceramide from serine and palmitoyl-CoA (FIG. 1 and FIG. 3). Indeed, increases in de novo synthesis could be responsible for the increase in dihydrosphingosine observed in both GBA KO cell lines.

An elevation in glucosylsphingosine levels was also observed, albeit a much smaller change in mass compared to GlcCer. Little is known about the metabolism of glucosylsphingosine, but several recent studies indicate that it might serve as a biomarker for Gaucher disease, a lysosomal storage disorder characterized by mutations in GBA (Dekker et al., 2011; Rolfs et al., 2013; Murugesan et al., 2016). The data presented herein suggested that GBA might catabolize both GlcCer and glucosylsphingosine (FIG. 3), suggesting a more global role for GBA in sphingolipid biology and pathophysiology.

Even though GBA could be a major regulator of influenza infectivity, other GlcCer metabolizing enzymes cannot be excluded. A previous study showed that glucosylceramide synthase (UGCG) is required for infection by one type of bunyavirus (severe fever with thrombocytopenia syndrome virus), but not for another (Rift Valley) or for other enveloped viruses tested (VSV and EBOV; Drake et al., 2017). Consistent with the present disclosure, these prior findings point to an optimal level of GlcCer being important for (certain) viral infections. Yet, the findings presented herein expand a need for optimal levels of GlcCer to other, potentially many, enveloped viruses that enter cells through late endosomes as well as to important endogenous endocytic cargo including EGF and its receptor.

It is well established that lipids are heterogeneously distributed throughout cells (Laude & Prior, 2004). Specialized lipid microdomains in various membrane compartments facilitate cellular organelle function and organization, including in endosomes (Helms & Zurzolo, 2004). Sphingolipids in particular have been shown to be enriched in endosomes (Urade et al., 1988), and perturbations to the sphingolipid pathway result in abnormal endosome size, location, and function (Lloyd-Evans et al., 2008; Yonamine et al., 2011; Pepperl et al., 2013; Lima et al., 2017). GlcCer has been implicated in altering the physical properties of membranes, for example, increased GlcCer results in decreased membrane fluidity (de Almeida et al., 2003; Varela et al., 2016). In addition, recent work demonstrated that cells taken from patients with Gaucher disease displayed restricted lateral lipid mobility and exhibited reduced rates of transferrin receptor endocytosis (Batta et al., 2018). Coupled with the present findings, these reports suggest that the disruption in endocytosis that was observed in GBA KO cells for critical cargos including pathogenic viruses and growth factor receptors could be due in part to an alteration in the biophysical properties of cellular membranes. Hence, targeting GBA might prove beneficial as part of strategies to ameliorate viral infections that utilize the endocytic pathway.

The findings disclosed herein thus suggested that GBA and optimal levels of GlcCer were required to regulate trafficking along the endocytic pathway of viruses and endogenous cargos, particularly to later stages of the pathway. Consequently, when GBA was missing, endocytosed enveloped viruses showed diminished fusion, entry, and infection, and critical growth factor receptors were not degraded, a process required for proper growth control. Collectively the data presented in FIGS. 7-11 indicated that the defect in viral entry (and consequent infection) and EGFR degradation seen in GBA KO cells was not due to lowered levels of cathepsins or impaired endosome acidification, but rather to a defect in trafficking of cargos to degradative endosomes/lysosomes.

As disclosed herein, it was determined that deleting GBA1, the enzyme that converts GlcCer (the primary product of UGCG enzymatic activity) to ceramide (FIG. 12) increased GlcCer levels and impairs endosome trafficking and influenza virus entry (Drews et al., 2019). Additionally, it was determined that knocking out UGCG decreases GlcCer levels (in both HEK 293 and A549 cells), impairing the entry of endosome-entering viruses. This is consistent with a requirement for specific optimal, highly-regulated amounts of GlcCer (as opposed to a threshold amount) for optimal endocytic trafficking. The presently disclosed data are also consistent with a previous study that found that inhibition of either UGCG or GBA led to mistargeting of glycosphingolipids from the Golgi to lysosomes (Sillence, 2002).

From this, it appeared that a homeostatic concentration of sphingolipids was necessary to maintain optimal infection of influenza virus, and that disruption of the balance between the production and consumptions of GlcCer could be responsible for the reduction in influenza virus infection observed in all of the GBA and UGCG KO cell lines tested. In support of the proposed GlcCer homeostasis model, examination of the lipid profiles of GBA and UGCG KO cells yielded one consistent theme: of all of the lipids analyzed, only GlcCer was consistently increased (in the case of GBA KOs) or decreased (in the case of UGCG KOs). Thus, there appeared to be connection between homeostatic levels of GlcCer and optimal cellular endocytosis.

The importance of maintaining the right balance of key molecules has been shown in several other cellular systems. For instance, tightly regulated signaling pathways often require a homeostatic state to maintain proper cell function (such as Notch signaling), and both up- and down-regulation of specific proteins has been shown to cause constitutive Akt signaling (Wang et al., 2014; Braune & Lendahl, 2016). In addition, GlcCer serves as the foundational lipid for a variety of higher order glycolipids, including lactosylceramide, GM3, and GMD3. Disruptions in GlcCer levels might result in disruptions in the homeostasis of any number of gangliosides, many of which have been implicated in a number of cellular functions, including maintenance of lipid rafts (Yu et al., 2011). For a complete analysis of the effects of knocking out GlcCer metabolizing enzymes on influenza virus infection these long chain gangliosides must be considered as well as the numerous sphingolipids listed in Table 4.

Because ceramide is a pro-apoptotic molecule (Shaw et al., 2018), cells limit any build-up of this bioactive lipid by converting it to either sphingomyelin or sphingosine/S1P. In HEK 293 UGCG KO cells, sphingomyelin levels were comparable to those in WT cells, but sphingosine-1-phosphate levels increased 14-fold, indicating that blocking GlcCer production by removing UGCG might shunt sphingolipid production to sphingosine-1-phosphate. Interestingly, sphingosine-1-phosphate was recently shown to heighten cellular susceptibility to influenza virus infection and as such might have masked part of the effect of knocking out UGCG on influenza virus entry (Seo et al., 2010; Seo et al., 2013). A549 UGCG KOs displayed a modest increase in sphingosine-1-phosphate levels as well as a larger increase in sphingomyelin, the most prevalent sphingolipid found in cells and predominantly localized to the plasma membrane (Tanguchi & Okazaki, 2014). Thus, it appeared that the elevated sphingomyelin levels in A549 UGCG KO cells could contribute to the increase in entry mediated by the glycoproteins of measles virus seen in A549 UGCG KO cells (FIG. 17B), as measles virus enters cells by fusion with the plasma-membrane (Rasbach et al., 2013).

It further appeared that the differences in sphingolipid compensation between HEK 293 and A549 UGCG KO cells might have been due to differences in their basal levels of various sphingolipid enzymes, such as sphingomyelin synthase or sphingosine kinase. It is important to note that any perturbations of sphingolipid levels could have an effect on influenza virus infection.

While it is possible that the lack of glycosphingolipids contributed to diminished influenza virus entry, compensatory ceramide metabolites like sphingomyelin or SIP might also contribute to this defect.

In summary, the findings presented herein demonstrated a previously undiscovered role for UGCG in influenza virus entry. By both pharmacological inhibition and genetic ablation, cells deficient in UGCG activity were found to display reduced entry and infection by influenza virus as well as entry mediated by the glycoproteins of other endosome-entering viruses including EBOV. These reductions in viral entry could have been due to a general impediment of normal endocytic trafficking, for example supported by the investigations of GlcCer metabolizing enzymes disclosed herein. For example, the present disclosure demonstrated that GBA was needed at a post binding and internalization step for proper trafficking of both EGF and influenza virus particles to late endosomes. A role for UGCG in infections by specific bunyaviruses at a post-internalization step was recently demonstrated (Drake et al., 2017), which also ruled out the need for UGCG for virus binding or internalization. That UGCG might be involved in endosome trafficking or virus-endosome fusion is also consistent with the data presented herein. These collective results suggested that UGCG plays a role in viral entry and might serve as a potential novel target for anti-influenza therapies.

### REFERENCES

All references listed below, as well as all references cited in the instant disclosure, including but not limited to all patents, patent applications and publications thereof, scientific journal articles, and database entries (e.g., GENBANK® and UniProt biosequence database entries and all annotations available therein) are incorporated herein by reference in their entireties to the extent that they supplement, explain, provide a background for, or teach methodology, techniques, and/or compositions employed herein.
Banerjee et al. (2013) High-Content Analysis of Sequential Events during the Early Phase of Influenza A Virus Infection. PLoS ONE 8:e68450.
Bär et al. (2006) Detection of Cell-Cell Fusion Mediated by Ebola Virus Glycoproteins. Journal of Virology 80:2815-2822.
Batishchev et al. (2016) pH-Dependent Formation and Disintegration of the Influenza A Virus Protein Scaffold To Provide Tension for Membrane Fusion. J Virol 90:575-585.
Batta et al. (2018) Alterations in the properties of the cell membrane due to glycosphingolipid accumulation in a model of Gaucher disease. Scientific Reports 8:157.
Braune & Lendahl (2016) Notch—a Goldilocks Signaling Pathway in Disease and Cancer Therapy. Discovery Medicine. 21:189-196.
Dawood et al. (2012) Estimated global mortality associated with the first 12 months of 2009 pandemic influenza A H1N1 virus circulation: a modelling study. The Lancet Infectious Diseases 12:687-695.
de Almeida et al. (2003) Sphingomyelin/Phosphatidylcholine/Cholesterol Phase Diagram: Boundaries and Composition of Lipid Rafts. Biophys J 85:2406-2416.
Dekker et al. (2011) Elevated plasma glucosylsphingosine in Gaucher disease: relation to phenotype, storage cell markers, and therapeutic response. Blood 118:e118-e127.
Drake et al. (2017) A role for glycolipid biosynthesis in severe fever with thrombocytopenia syndrome virus entry. PLOS Pathogens 13:e1006316.
Dreschers et al. (2007) Infections with human rhinovirus induce the formation of distinct functional membrane domains. Cell Physiol Biochem 20:241-254.
Drews et al. (2019) Glucosylceramidase Maintains Influenza Infection By Regulating Endocytosis. Journal of Virology. 93(12):JVI.00017-19.
Duncan et al. (2009) Neisseria gonorrhoeae activates the proteinase Cathepsin B to mediate the signaling activities of the NLRP3 and ASC-containing inflammasome. J Immunol 182:6460-6469.
Ebert et al. (2002) Cathepsin L and Cathepsin B Mediate Reovirus Disassembly in Murine Fibroblast Cells. J Biol Chem 277:24609-24617.
Eriksson et al. (2017) Analysis of Lysosomal pH by Flow Cytometry Using FITC-Dextran Loaded Cells, p. 179-189. In Öllinger, K, Appelqvist, H (eds.), Lysosomes: Methods and Protocols. Springer New York, N.Y., New York, United States of America.
Finnegan et al. (2007) Sphingomyelinase Restricts the Lateral Diffusion of CD4 and Inhibits Human Immunodeficiency Virus Fusion. J Virol 81:5294-5304.
Gault et al. (2010) An overview of sphingolipid metabolism: from synthesis to breakdown. Adv Exp Med Biol 688:1-23.
Gerl et al. (2012) Quantitative analysis of the liposomes of the influenza virus envelope and MDCK cell apical membrane. J Cell Biol 196:213-221.
Gillespie et al. (2013) Selective inhibitor of endosomal trafficking pathways exploited by multiple toxins and viruses. PNAS 110:E4904-E4912.
Gotoh et al. (1990) An endoprotease homologous to the blood clotting factor X as a determinant of viral tropism in chick embryo. EMBO J 9:4189-4195.
Gotoh et al. (1992) Isolation of factor Xa from chick embryo as the amniotic endoprotease responsible for paramyxovirus activation. FEBS Letters 296:274-278.
Grassmé et al. (2005) Rhinoviruses Infect Human Epithelial Cells via Ceramide-enriched Membrane Platforms. J Biol Chem 280:26256-26262.
Grazide et al. (2004) Cytoprotective effect of glucosylceramide synthase inhibition against daunorubicin-induced apoptosis in human leukemic cell lines. J Biol Chem. 279:18256-18261.
Grove & Marsh (2011) The cell biology of receptor-mediated virus entry. J Cell Biol 195:1071-1082.
Hankins et al. (2011) Exogenous Ceramide-1-phosphate Reduces Lipopolysaccharide (LPS)-mediated Cytokine Expression. J Biol Chem 286:44357-44366.
Helms & Zurzolo (2004) Lipids as targeting signals: lipid rafts and intracellular trafficking. Traffic 5:247-254.
Hoenen et al. (2014) Modeling The Lifecycle Of Ebola Virus Under Biosafety Level 2 Conditions With Virus-like Particles Containing Tetracistronic Minigenomes. J Vis Exp.
Hufford et al. (2012) Influenza-Infected Neutrophils within the Infected Lungs Act as Antigen Presenting Cells for Anti-Viral CD8+ T Cells. PLoS One 7.
Ichikawa & Hirabayashi (1998) Glucosylceramide synthase and glycosphingolipid synthesis. Trends in Cell Biology. 8:198-202.
Imai & Kawaoka (2012) The role of receptor binding specificity in interspecies transmission of influenza viruses. Current Opinion in Virology 2:160-167.
Ishibashi et al. (2013) New insights on glucosylated lipids: Metabolism and functions. Biochimica et Biophysica Acta (BBA)—Molecular and Cell Biology of Lipids 1831:1475-1485.
Jan et al. (2000) Sindbis virus entry into cells triggers apoptosis by activating sphingomyelinase, leading to the release of ceramide. J Virol 74:6425-6432.
Johansen et al. (2013) FDA-Approved Selective Estrogen Receptor Modulators Inhibit Ebola Virus Infection. Science Translational Medicine 5:190ra79-190ra79.
Kanj et al. (2006) Ceramide regulates SR protein phosphorylation during adenoviral infection. Virology 345:280-289.
Konan & Sanchez-Felipe (2014) Lipids and RNA virus replication. Curr Opin Virol 9:45-52.
Laude & Prior (2004) Plasma membrane microdomains: organisation, function and trafficking. Mol Membr Biol 21:193-205.
Li et al. (2014) pH-Controlled Two-Step Uncoating of Influenza Virus. Biophys J 106:1447-1456.
Libersou et al. (2010) Distinct structural rearrangements of the VSV glycoprotein drive membrane fusion. The Journal of Cell Biology 191:199-210.
Lima et al. (2017) Sphingosine and Sphingosine Kinase 1 Involvement in Endocytic Membrane Trafficking. J Biol Chem jbc.M116.762377.
Lloyd-Evans et al. (2008) Niemann-Pick disease type C1 is a sphingosine storage disease that causes deregulation of lysosomal calcium. Nature Medicine 14:1247-1255.
Lozach et al. (2011) Late-penetrating viruses. Current Opinion in Virology 1:35-43.
Marsh & Helenius (2006) Virus Entry: Open Sesame. Cell 124:729-740.
Marvin et al. (2017) Influenza Virus Overcomes Cellular Blocks To Productively Replicate, Impacting Macrophage Function. J Virol 91:e01417-16.
Miller et al. (2012) Ebolavirus Requires Acid Sphingomyelinase Activity and Plasma Membrane Sphingomyelin for Infection. Journal of Virology 86:7473-7483.
Murugesan et al. (2016) Glucosylsphingosine is a key biomarker of Gaucher disease. Am J Hematol 91:1082-1089.
Nelson et al. (2016) Clomiphene and Its Isomers Block Ebola Virus Particle Entry and Infection with Similar Potency: Potential Therapeutic Implications. Viruses 8.
Ochiai et al. (1995) Inhibitory effect of bafilomycin A1, a specific inhibitor of vacuolar-type proton pump, on the growth of influenza A and B viruses in MDCK cells. Antiviral Research 27:425-430.
Opresko et al. (1995) Endocytosis and Lysosomal Targeting of Epidermal Growth Factor Receptors Are Mediated by Distinct Sequences Independent of the Tyrosine Kinase Domain. J Biol Chem 270:4325-4333.
PCT International Patent Application Publication Nos. WO 2005/098014; WO 2008/150486; WO 2010/014554; WO 2010/091164; WO 2011/133915; WO 2012/129084; WO 2014/043068; WO 2014/151291; WO 2015/089067; WO 2015/173168; WO 2015/173169.
Pepperl et al. (2013) Sphingolipid depletion impairs endocytic traffic and inhibits Wingless signaling. Mechanisms of Development 130:493-505.
Pewzner-Jung et al. (2010) A Critical Role for Ceramide Synthase 2 in Liver Homeostasis. J Biol Chem 285:10911-10923.
Puri et al. (2004) An inhibitor of glycosphingolipid metabolism blocks HIV-1 infection of primary T-cells. AIDS 18:849.
Rasbach et al. (2013) The Receptor Attachment Function of Measles Virus Hemagglutinin Can Be Replaced with an Autonomous Protein That Binds Her2/neu While Maintaining Its Fusion-Helper Function. J Virol. 87:6246-6256.
Rawat et al. (2004) Elevated Expression of GM3 in Receptor-Bearing Targets Confers Resistance to Human Immunodeficiency Virus Type 1 Fusion. J Virol 78:7360-7368.
Rolfs et al. (2013) Glucosylsphingosine Is a Highly Sensitive and Specific Biomarker for Primary Diagnostic and Follow-Up Monitoring in Gaucher Disease in a Non-Jewish, Caucasian Cohort of Gaucher Disease Patients. PLOS ONE 8:e79732.
Ryland et al. (2013) C6-Ceramide Nanoliposomes Target the Warburg Effect in Chronic Lymphocytic Leukemia. PLOS ONE. 8:e84648.
Rosenbloom & Weinreb (2013) Gaucher Disease: A Comprehensive Review. CRO 18.
Sakai et al. (2006) Dual Wavelength Imaging Allows Analysis of Membrane Fusion of Influenza Virus inside Cells. J Virol 80:2013-2018.
Sakai et al. (2017) Influenza A virus hemagglutinin and neuraminidase act as novel motile machinery. Scientific Reports 7:45043.
Schneider-Schaulies & Schneider-Schaulies (2015) Sphingolipids in viral infection. Biological Chemistry 396:585-595.
Schulze et al. (2009) Principles of lysosomal membrane degradation: Cellular topology and biochemistry of lysosomal lipid degradation. Biochimica et Biophysica Acta (BBA)—Molecular Cell Research 1793:674-683.
Seo et al. (2010) Sphingosine 1-Phosphate-Metabolizing Enzymes Control Influenza Virus Propagation and Viral Cytopathogenicity. J Virol 84:8124-8131.
Seo et al. (2013) Sphingosine Kinase 1 Serves as a Pro-Viral Factor by Regulating Viral RNA Synthesis and Nuclear Export of Viral Ribonucleoprotein Complex upon Influenza Virus Infection. PLoS One 8.
Shaw et al. (2018) Novel Sphingolipid-Based Cancer Therapeutics in the Personalized Medicine Era, p. 327-366. In Chalfant, CE, Fisher, PB (eds.), Advances in Cancer Research. Academic Press.
Shivanna et al. (2015) Ceramide formation mediated by acid sphingomyelinase facilitates endosomal escape of caliciviruses. Virology 483:218-228.
Sieczkarski & Whittaker (2003) Differential Requirements of Rab5 and Rab7 for Endocytosis of Influenza and Other Enveloped Viruses. Traffic 4:333-343.
Sillence (2002) Glucosylceramide modulates membrane traffic along the endocytic pathway. The Journal of Lipid Research 43:1837-1845.
Soudani et al. (2019) Ceramide Suppresses Influenza A Virus Replication In Vitro. Journal of Virology. 93:e00053-19.
Stirnemann et al. (2017) A Review of Gaucher Disease Pathophysiology, Clinical Presentation and Treatments. Int J Mol Sci 18.
Sun et al. (2010) Modifications to the Hemagglutinin Cleavage Site Control the Virulence of a Neurotropic H1N1 Influenza Virus. J Virol 84:8683-8690.
Tafesse et al. (2013) Intact sphingomyelin biosynthetic pathway is essential for intracellular transport of influenza virus glycoproteins. Proc Natl Acad Sci USA 110:6406-6411.
Tani et al. (2010) Involvement of Ceramide in the Propagation of Japanese Encephalitis Virus. J Virol 84:2798-2807.
Taniguchi & Okazaki (2014) The role of sphingomyelin and sphingomyelin synthases in cell death, proliferation and migration—from cell and animal models to human disorders. Biochimica et Biophysica Acta (BBA)—Molecular and Cell Biology of Lipids. 1841:692-703.
Tatsuo et al. (2000) Virus Entry Is a Major Determinant of Cell Tropism of Edmonston and Wild-Type Strains of Measles Virus as Revealed by Vesicular Stomatitis Virus Pseudotypes Bearing Their Envelope Proteins. Journal of Virology 74:4139-4145.
Tscherne et al. (2010) An enzymatic virus-like particle assay for sensitive detection of virus entry. Journal of Virological Methods 163:336-343.
Turikovi et al. (2014) New inhibitors of glucosylceramide synthase and their effect on cell fate. Acta Chimica Slovaca. 7:99-104.
Urade et al. (1988) Endosomes differ from plasma membranes in the phospholipid molecular species composition. Biochim Biophys Acta 946:151-163.
U.S. Patent Application Publication Nos. 2010/0151528; 2014/0255381; 2014/0336174; and 2019/0248790.
U.S. Pat. Nos. 4,486,602; 6,916,802; 7,196,205; 7,253,185; 7,365,192; 7,501,493; 7,615,573; 8,168,587; 8,309,593; 8,729,075; 8,940,776; 8,961,959; 9,126,993; 9,481,700; and 9,718,777.
Varela et al. (2016) Pathological levels of glucosylceramide change the biophysical properties of artificial and cell membranes. Phys Chem Chem Phys 19:340-346.
Wang et al. (2014) Both Decreased and Increased SRPK1 Levels Promote Cancer by Interfering with PHLPP-Mediated Dephosphorylation of Akt. Molecular Cell. 54:378-391.
Wegner et al. (2018) The UDP-glucose ceramide glycosyltransferase (UGCG) and the link to multidrug resistance protein 1 (MDR1). BMC Cancer. 18:Article 153.
Weng et al. (2010) Sphingomyelin Activates Hepatitis C Virus RNA Polymerase in a Genotype-Specific Manner. J Virol 84:11761-11770.
White & Whittaker (2016) Fusion of Enveloped Viruses in Endosomes. Traffic 17:593-614.
White et al. (1982) Membrane fusion activity of influenza virus. EMBO J 1:217-222.
White et al. (2008) Structures and Mechanisms of Viral Membrane Fusion Proteins. Crit Rev Biochem Mol Biol 43:189-219.
Whitt (2010) Generation of VSV Pseudotypes Using Recombinant ΔG-VSV for Studies on Virus Entry, Identification of Entry Inhibitors, and Immune Responses to Vaccines. J Virol Methods 169:365-374.
Yamauchi & Helenius (2013) Virus entry at a glance. J Cell Sci 126:1289-1295.
Yeganeh et al. (2015) Suppression of influenza A virus replication in human lung epithelial 776 cells by noncytotoxic concentrations bafilomycin A1. Am J Physiol Lung Cell Mol Physiol 308:L270-L286.
Yonamine et al. (2011) Sphingosine kinases and their metabolites modulate endolysosomal trafficking in photoreceptors. The Journal of Cell Biology 192:557-567.
Yu et al. (2011) Structures, biosynthesis, and functions of gangliosides—An overview. J Oleo Sci. 60:537-544.

While the presently disclosed subject matter has been disclosed with reference to specific embodiments, it is apparent that other embodiments and variations of the presently disclosed subject matter may be devised by others skilled in the art without departing from the true spirit and scope of the presently disclosed subject matter.

1. A method for treating and/or inhibiting a viral infection in a subject, the method comprising administering to a subject infected with and/or at risk for infection with a virus a composition comprising a glucosylceramidase inhibitor, a glucosylceramide synthase inhibitor, or any combination thereof via a route and in an amount effective for treating and/or inhibiting the viral infection in the subject.

2. The method of claim 1, wherein the viral infection is from a virus selected from the group consisting of an influenza A virus, an influenza B virus, an influenza C virus, a vesicular stomatitis virus (VSV), an Ebola virus (EBOV), a measles virus, a coronavirus, optionally COVID-19, and any combination thereof.

3. The method of claim 1, wherein the glucosylceramidase inhibitor is a small molecule, an anti-glucosylceramidase antibody, an inhibitory nucleic acid that targets a glucosylceramidase gene product, or any combination thereof.

4. The method of claim 3, wherein the glucosylceramidase inhibitor is selected from the group consisting of conduritol b epoxide, D-threo-1-phenyl-2-palmitoylamino-3-pyrrolidino-1-propanol (P4), castanospermine ((1S,6S,7R,8R,8aR)-octahydroindolizine-1,6,7,8-tetrol), isofagomine (5R-(hydroxymethyl)-3R,4R-piperidinediol, mono 2S,3S-dihydroxybutanedioate), valienamine, ((1S,2S,3R,6S)-6-Amino-4-(hydroxymethyl)cyclohex-4-ene-1,2,3-triol), validamine, ((1R,2S,3S,4S,6R)-4-amino-6-(hydroxymethyl)cyclohexane-1,2,3-triol), derivatives and salts thereof, and/or combinations thereof.

5. The method of claim 1, wherein the glucosylceramide synthase inhibitor is a small molecule, an anti-glucosylceramide synthase antibody, an inhibitory nucleic acid that targets a glucosylceramide synthase gene product, or any combination thereof.

6. The method of claim 5, wherein the glucosylceramide synthase inhibitor is selected from the group consisting of an iminosugar, N-butyl-deoxynojirimycin (miglustat), [(3S)-1-azabicyclo[2.2.2]octan-3-yl] N-[2-[2-(4-fluorophenyl)-1,3-thiazol-4-yl]propan-2-yl]carbamate (ibiglustat), N-[(1R,2R)-1-(2,3-Dihydro-1,4-benzodioxin-6-yl)-1-hydroxy-3-(1-pyrrolidinyl)-2-propanyl]octanamide (eliglustat), 1-phenyl-2-palmitoylamino-3-morpholino-1-propanol, and derivatives and/or combinations thereof.

7. The method of claim 1, wherein the subject is a human.

8. A method for inhibiting infection of a cell with a virus, the method comprising contacting the cell with a composition comprising a glucosylceramidase inhibitor, a glucosylceramide synthase inhibitor, or any combination thereof in an amount sufficient for inhibiting infection of the cell with the virus.

9. The method of claim 8, wherein the virus selected from the group consisting of an influenza A virus, an influenza B virus, an influenza C virus, a vesicular stomatitis virus (VSV), an Ebola virus (EBOV), a measles virus, a coronavirus, optionally COVID-19, and any combination thereof.

10. The method of claim 8, wherein the glucosylceramidase inhibitor is a small molecule, an anti-glucosylceramidase antibody, an inhibitory nucleic acid that targets a glucosylceramidase gene product, or any combination thereof.

11. The method of claim 10, wherein the glucosylceramidase inhibitor is selected from the group consisting of conduritol b epoxide (i.e., a mixture of 1-L-1,2-anhydro-myo-inositol and 1-D-1,2-anhydro-myo-inositol), D-threo-1-phenyl-2-palmitoylamino-3-pyrrolidino-1-propanol (P4; U.S. Pat. No. 8,168,587), castanospermine ((1S,6S,7R,8R,8aR)-octahydroindolizine-1,6,7,8-tetrol), isofagomine (5R-(hydroxymethyl)-3R,4R-piperidinediol, mono 2S,3S-dihydroxybutanedioate), valienamine, ((1S,2S,3R,6S)-6-Amino-4-(hydroxymethyl)cyclohex-4-ene-1,2,3-triol), validamine, ((1R,2S,3S,4S,6R)-4-amino-6-(hydroxymethyl)cyclohexane-1,2,3-triol), derivatives and salts thereof, and/or combinations thereof.

12. The method of claim 8, wherein the glucosylceramide synthase inhibitor is a small molecule, an anti-glucosylceramide synthase antibody, an inhibitory nucleic acid that targets a glucosylceramide synthase gene product, or any combination thereof.

13. The method of claim 12, wherein the glucosylceramide synthase inhibitor is selected from the group consisting of an iminosugar, N-butyl-deoxynojirimycin (miglustat), [(3S)-1-azabicyclo[2.2.2]octan-3-yl] N-[2-[2-(4-fluorophenyl)-1,3-thiazol-4-yl]propan-2-yl]carbamate (ibiglustat), N-[(1R,2R)-1-(2,3-Dihydro-1,4-benzodioxin-6-yl)-1-hydroxy-3-(1-pyrrolidinyl)-2-propanyl]octanamide (eliglustat), 1-phenyl-2-palmitoylamino-3-morpholino-1-propanol, and derivatives and/or combinations thereof.

14. The method of claim 8, wherein the cell is a human cell, optionally a human cell present within a subject.

15. A method for inhibiting endosomal fusion of a virus in a cell, the method comprising contacting a cell with a glucosylceramidase inhibitor, a glucosylceramide synthase inhibitor, or any combination thereof, wherein an amount of the glucosylceramidase inhibitor, the glucosylceramide synthase inhibitor, or the combination thereof is effective for inhibiting endosomal fusion of the virus in the cell.

16. The method of claim 15, wherein the virus selected from the group consisting of an influenza A virus, an influenza B virus, an influenza C virus, a vesicular stomatitis virus (VSV), an Ebola virus (EBOV), a measles virus, a coronavirus, optionally COVID-19, and any combination thereof.

17. The method of claim 15, wherein:
(i) the glucosylceramidase inhibitor is selected from the group consisting of conduritol b epoxide (i.e., a mixture of 1-L-1,2-anhydro-myo-inositol and 1-D-1,2-anhydro-myo-inositol), D-threo-1-phenyl-2-palmitoylamino-3-pyrrolidino-1-propanol (P4; U.S. Pat. No. 8,168,587), castanospermine ((1S,6S,7R,8R,8aR)-octahydroindolizine-1,6,7,8-tetrol), isofagomine (5R-(hydroxymethyl)-3R,4R-piperidinediol, mono 2S,3S-dihydroxybutanedioate), valienamine, ((1S,2S,3R,6S)-6-Amino-4-(hydroxymethyl)cyclohex-4-ene-1,2,3-triol), validamine, ((1R,2S,3S,4S,6R)-4-amino-6-(hydroxymethyl)cyclohexane-1,2,3-triol), derivatives and salts thereof, and/or combinations thereof; and/or
(ii) the glucosylceramide synthase inhibitor is a small molecule, an anti-glucosylceramide synthase antibody, an inhibitory nucleic acid that targets a glucosylceramide synthase gene product, derivatives and salts thereof, and/or combinations thereof, optionally, wherein the glucosylceramide synthase inhibitor is selected from the group consisting of an iminosugar, N-butyl-deoxynojirimycin (miglustat), [(3S)-1-azabicyclo[2.2.2]octan-3-yl] N-[2-[2-(4-fluorophenyl)-1,3-thiazol-4-yl]propan-2-yl]carbamate (ibiglustat), N-[(1R,2R)-1-(2,3-Dihydro-1,4-benzodioxin-6-yl)-1-hydroxy-3-(1-pyrrolidinyl)-2-propanyl]octanamide (eliglustat), 1-phenyl-2-palmitoylamino-3-morpholino-1-propanol, and derivatives and/or combinations thereof.

18. The method of claim 15, wherein the cell is a human cell, optionally a human cell present within a subject.

19-24. (canceled)
